(19) United States
(12) Patent Application Publication
(10) Pub. No.: US 2024/0307557 A1
(43) Pub. Date: Sep. 19, 2024

(54) THERAPEUTIC GENE SILENCING WITH CRISPR-CAS13

(71) Applicant: THE BOARD OF TRUSTEES OF THE UNIVERSITY OF ILLINOIS, URBANA, IL (US)

(72) Inventors: THOMAS GAJ, URBANA, IL (US); COLIN LIM, CHAMPAIGN, IL (US); JACKSON POWELL, URBANA, IL (US)

(73) Assignee: THE BOARD OF TRUSTEES OF THE UNIVERSITY OF ILLINOIS, URBANA, IL (US)

(21) Appl. No.: 18/576,407

(22) PCT Filed: Jul. 8, 2022
(86) PCT No.: PCT/US22/36494

§ 371 Date: Jan. 4, 2024

Related U.S. Application Data
(60) Provisional application No. 63/220,385, filed on Jul. 9, 2021.

(51) Int. Cl. A61K 48/00; A61K 9/00; A61P 21/00; A61P 25/28; C12N 9/22; C12N 15/11; C12N 15/86

(52) U.S. Cl. CPC A61K 48/005; A61K 9/0019; A61K 9/0085; A61K 48/0075; A61K 48/0083; A61P 21/00; A61P 25/28; C12N 9/22; C12N 15/11; C12N 15/86; C12N 2310/20; C12N 2750/14143; C12N 2830/50

(57) ABSTRACT

Provided herein are compositions and methods for suppressing mutant gene function using Cas13 nucleases that can be delivered to the spinal cord and brain to mediate the knockdown of genes that are causative for autosomal dominant neurodegenerative disorders.

### PRIORITY

This application claims the benefit of U.S. Ser. No. 63/220,385, filed on Jul. 9, 2021, which is incorporated herein by reference in its entirety.

### BACKGROUND

Clustered Regularly Interspaced Short Palindromic Repeats (CRISPR) and their CRISPR associated (Cas) proteins are a diverse family of adaptive bacterial immune systems that have been successfully co-opted to enable efficient, sequence-specific genome editing in mammalian cells. While CRISPR technology is most commonly associated with DNA editing, the discovery of Cas13 effector proteins that can cleave single-stranded RNA via an intrinsic RNase activity has facilitated the creation of a new, highly programmable toolbox for targeting RNA.

### SUMMARY

Provided herein are recombinant adeno-associated virus (rAAV) vectors. The vectors can comprise in 5′ to 3′ direction: a first AAV inverted terminal repeat (ITR) sequence; a crRNA sequence having homology to a superoxide dismutase 1 (SOD1) gene or a huntingtin (HTT) gene; and a second AAV ITR sequence. The vectors can further comprise, between the first AAV ITR and the second AAV ITR sequence: a promoter sequence; a nucleic acid molecule encoding a Cas13 polypeptide; and a polyA sequence. An rAAV vector can further comprise one or more nuclear localization signals (NLSs). The one or more NLSs can occur 5′ to the nucleic acid molecule encoding the Cas13 polypeptide, 3′ to the nucleic acid molecule encoding the Cas13 polypeptide, or both. The promoter can be a cytomegalovirus early enhancer/chicken β-actin (CAG) promoter. A vector can further comprise one or more human influenza hemagglutinin (HA) epitope tags. The polyA sequence can be a bovine growth hormone (BGH) polyA sequence. The crRNA sequence can comprise one or more of SEQ ID NOs:58-71. The crRNA can further comprise a Cas13-specific direct repeat region. A Cas13-specific direct repeat region can comprise the sequence set forth in SEQ ID NO:90. The Cas13 polypeptide can be a Cas13d polypeptide. The Cas13 polypeptide can be a Ruminococcus flavefaciens Cas13d (RfxCas13d) polypeptide. The Cas13 polypeptide can be a catalytically deactivated Cas13 (dCas13) polypeptide. A dCas13 polypeptide can be a dCas13d polypeptide. A promoter can be associated with the crRNA sequence having homology to a superoxide dismutase 1 (SOD1) gene or a huntingtin (HTT) gene, such that the promoter drives expression of the crRNA sequence.

An rAAV vector can further comprise a nucleic acid molecule encoding an AAV capsid protein. The AAV capsid protein can be an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein or an AAVrh. 10 capsid protein.

Also provided herein are pharmaceutical compositions comprising any rAAV vector described herein and at least one pharmaceutically acceptable excipient and/or additive.

Another aspect provides a method for treating a subject having a disease and/or disorder involving an SOD1 gene or an HTT gene. The method can comprise administering to the subject at least one therapeutically effective amount of any rAAV vector or pharmaceutical composition described herein. The disease and/or disorder involving the SOD1 gene can be amyotrophic lateral sclerosis (ALS), familial amyotrophic lateral sclerosis, or Parkinson's disease, and the disease and/or disorder involving the HTT gene can be Huntington's disease (HD). The rAAV viral vector or the pharmaceutical composition can be administered to the subject at a dose ranging from about 10¹¹ to about 10¹⁸ viral vector particles. The rAAV viral vector or the pharmaceutical composition can be administered to the subject at a dose ranging from about 10¹³ to about 10¹⁶ viral vector particles. The rAAV viral vector or the pharmaceutical composition can be administered to the subject intravenously, intrathecally, intrastriatally, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally or intranerve.

Yet another aspect provides an rAAV vector pharmaceutical composition as described herein for use in treating a disease and/or disorder involving an SOD1 or HTT gene in a subject in need thereof.

Still another aspect provides the use of an rAAV viral vector or pharmaceutical composition as described herein. The disease and/or disorder can involve the SOD1 gene and can be amyotrophic lateral sclerosis (ALS), familial amyotrophic lateral sclerosis, and Parkinson's disease. The disease and/or disorder involving the HTT gene can be HD. The rAAV viral vector or the pharmaceutical composition can be for administration to the subject at a dose ranging from about 10¹¹ to about 10¹⁸ viral vector particles. The rAAV viral vector or the pharmaceutical composition can be for administration to the subject at a dose ranging from about 10¹³ to about 10¹⁶ viral vector particles. The rAAV viral vector or the pharmaceutical composition can be for administration to the subject intravenously, intrathecally, intrastriatally, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally, or intranerve.

Another aspect provides a method of reducing an amount of mRNA encoding SOD1 or HTT in a cell comprising delivering the recombinant rAAV vectors described herein to the cell.

Even another aspect provides an isolated crRNA comprising one or more of the nucleotide sequences set forth in SEQ ID NOs: 58-71. The isolated crRNA can further comprise a Cas13-specific direct repeat region. The Cas13-specific direct repeat region can be that as set forth in SEQ ID NO:90. The isolated crRNA can further comprise a promoter sequence.

Therefore, provided herein is a general platform for suppressing mutant gene function in vivo, in vitro, and ex vivo. More specifically, as various neurological disorders arise from gain-of-function mechanism(s) that could benefit from gene silencing, we demonstrate that Cas13 can be delivered to the spinal cord and brain to mediate the knockdown of the specific genes causative for two autosomal dominant neurodegenerative disorders: amyotrophic lateral sclerosis (ALS), a motor neuron disease that can be caused by mutations in the protein superoxide dismutase 1 (SOD1) and Huntington's disease (HD), a disorder caused by an abnormal expansion of a polyglutamine tract in the huntingtin (HTT) protein.

### BRIEF DESCRIPTION OF THE DRAWINGS

FIGS. 1A-1F show an example in which RfxCas13d can be programmed to knockdown human SOD1. (FIG. 1A) Schematic illustrating the putative mechanism of RNA knockdown by RfxCas13d. RfxCas13d is directed to a target RNA by a CRISPR RNA (crRNA) guide molecule via RNA-RNA base complementarity. After binding to the target RNA sequence, the ribonuclease activity of RfxCas13d is activated, resulting in transcript cleavage and gene knockdown. (FIG. 1B) (Top) Schematic depicting the mature human SOD1 mRNA and the approximate locations of the crRNA binding sites (yellow bars). (Bottom) Sequences targeted by the crRNAs. crRNA1 is SEQ ID NO:58; crRNA2 is SEQ ID NO: 59; crRNA3 is SEQ ID NO:60; crRNA4 is SEQ ID NO:61; crRNA5 is SEQ ID NO:62; crRNA6 is SEQ ID NO:63; crRNA7 is SEQ ID NO:64; crRNA8 is SEQ ID NO:65; crRNA9 is SEQ ID NO:66; crRNA10 is SEQ ID NO:67. (FIG. 1C) Mean fluorescence intensity (MFI) of AcGFP in HEK293T cells 72 hr after transfection with SOD1 reporter plasmid and RfxCas13d with SOD1-targeting crRNAs. Data was normalized to cells transfected with RfxCas13d and non-targeted crRNA, denoted NTG (n=3). (FIG. 1D) Normalized SOD1 mRNA in HEK293T cells 72 hr after transfection with RfxCas13d and SOD1-targeting crRNA or NTG crRNA. Data were normalized to relative SOD1 mRNA in untreated cells (n=3). (FIG. 1E) Quantification of human SOD1 protein in cell lysate from HEK293T cells 72 hr after transfection with RtxCas13d and SOD1-targeting crRNA or NTG crRNA. SOD1 protein in each sample was normalized to β-actin protein (n=3). (FIG. 1F) Normalized SOD1 mRNA in HEK293T cells 72 hr after transfection with RfxCas13d, catalytically dead RfxCas13d or no RfxCas13d with SOD1-targeting crRNA or NTG crRNA. Data are normalized to relative SOD1 mRNA in untreated cells (n=3). Error bars indicate SEM. *P<0.05, **P<0.01, ***P<0.001; (FIG. 1C, FIG. 1D, FIG. 1E and FIG. 1F) two-tailed unpaired t-test.

FIGS. 2A-2J show an example in which RfxCas13d improves therapeutic outcomes in G93A-SOD1 mice. (FIG. 2A) Schematic of the AAV vector constructed for this study. Abbreviations are as follows: ITR, inverted terminal repeat; CAG, cytomegalovirus early enhancer/chicken β-actin promoter; NLS, nuclear localization signal; 3×HA, three tandem repeats of the human influenza hemagglutinin (HA) epitope tag. BGH, BGH-poly(A) polyadenylation signal. (FIG. 2B) Schematic depicting the timeline for the G93A-SOD1 study. (FIG. 2C) Percent before disease onset, (FIG. 2D) percent survival, (FIG. 2E) duration of disease (FIG. 2F) normalized rotarod and (FIG. 2G) normalized weight of G93A-SOD1 mice injected with 2×10¹¹ vector genomes (vg) of AAV9-RfxCas13d-hSOD1 or AAV9-RfxCas13d-NTG (n=15 for both groups for all measurements). (FIG. 2G) Grey box indicates late-stage disease phase. (FIG. 2H) Relative human SOD1 (hSOD1) protein from (left) lumbar, (middle) thoracic and (right) cervical spinal cord lysate (n=4 for both groups) and (FIG. 2I) relative hSOD1 mRNA (n=4 for both groups) from whole spinal cord lysate of G93A-SOD1 mice injected with 2×10¹¹ vg of AAV9-RfxCas13d-hSOD1 or AAV9-RfxCas13d-NTG. (FIG. 2H) hSOD1 protein for each sample was normalized to β-actin protein. (J FIG. 2) Representative immunofluorescence staining of the lumbar spinal cord of G93A-SOD1 mice injected AAV9-RfxCas13d-hSOD1. Scale bars, 500 μm, left; 30 μm, anterior horn. (FIG. 2F and FIG. 2G) Mean rotarod and weight values for each mouse were normalized to values from week nine for the same mouse. (FIG. 2G) Rate of weight loss was determined by a linear regression analysis. Values represent means and error bars indicate (FIG. 2F and FIG. 2G) SD or (FIG. 2H and FIG. 2I) SEM. *P<0.05, **P<0.01, ***P<0.001. (FIG. 2C and FIG. 2D) log-rank Mantel-Cox test; (FIG. 2E) one-tailed unpaired t-test; (FIG. 2F) two-way ANOVA followed by a Bonferroni post hoc test; (FIG. 2H and FIG. 2I) one-tailed unpaired t-test.

FIGS. 3A-3F show an example in which RfxCas13d can target human huntingtin. (FIG. 3A) (Top) Schematic depicting HTT exon 1 in the context of the reporter and the approximate locations of the crRNA binding sites (red bars). (Bottom) Sequences targeted by the crRNAs. crRNA1 is SEQ ID NO:68; crRNA2 is SEQ ID NO: 69; crRNA3 is SEQ ID NO:70; crRNA4 is SEQ ID NO:71. (FIG. 3B) Mean fluorescence intensity (MFI) of cyan fluorescent protein (CFP) in HEK293T cells 72 hr after transfection with HTT reporter and RfxCas13d with HTT-targeting crRNAs. Data was normalized to cells transfected with RtxCas13d and NIG crRNA (n=3). (FIG. 3C) (Top) Representative fluorescence microscopy image of HEK293T cells 72 hr after transfection with the HTT reporter and RfxCas13d with HTT crRNA 1. (Bottom). Quantification of HTT-CFP protein in HEK293T cells 72 hr after transfection with the HTT reporter and RfxCas13d with HTT-targeting crRNAs or a NTG crRNA. CFP protein in each sample was normalized to β-actin protein. (FIG. 3D) Normalized HTT mRNA in HEK293T cells 72 hr after transfection with RfxCas13d and HTT crRNAs. Data was normalized to relative HTT mRNA in cells transfected with RfxCas13d with NTG crRNA (n=3). (FIG. 3E) Volcano plot of differentially expressed genes (DEGs) by RNA-seq in HEK293T cells 72 hr after transfection with RfxCas13d and HTT crRNA as compared to untreated cells (n=3). HTT expression denoted by red circle. (FIG. 3F) Number of significant DEGs (>1.25-fold change, FDR P-value<0.01) by RNA-seq in HEK293T cells 72 hr after transfection with RfxCas13d alone, RfxCas13d with HTT crRNA, or RfxCas13d with NTG crRNA as compared to untreated cells (n=3). Error bars indicate SEM. ***P<0.001; (FIG. 3B, FIG. 3C, and FIG. 3D) one-tailed unpaired t-test.

FIGS. 4A-4D show an example in which RfxCas13d can reduce huntingtin protein in the brain of a mouse model of Huntington's disease. (FIG. 4A) Human HTT (hHTT) protein (n=4) and (FIG. 4B) hHTT mRNA (n=3) from striatal tissue four weeks after R6/2 mice were injected with 2×10¹⁰ vector genomes (vg) of AAV1-RfxCas13d-hHTT or AAV1-RfxCas13d-NTG. (FIG. 4A) hHTT protein in each sample was normalized to β-actin protein. (FIG. 4C) Representative immunofluorescence staining of striatal tissue four weeks after R6/2 mice were injected with either 2×10¹⁰ vg of AAV1-RfxCas13d-hHTT or AAV1-RfxCas13d-NTG. Scale bars, 30 μm. (FIG. 4D) Quantification of the percentage of RfxCas13d⁺ cells in with measurable HTT immunoreactive inclusions, as determined by immunofluorescence staining of the striatum four weeks after R6/2 mice were injected with 2×10¹⁰ vg of AAV1-RfxCas13d-hHTT or AAV1-RfxCas13d-NTG. >100 cells were counted per animal. Values represent means and error bars indicate SEM. *P<0.05, **P<0.01, ****P<0.0001. (FIG. 4A, FIG. 4B and FIG. 4D) one-tailed unpaired t-test.

FIG. 5 crRNAs between 28 to 32 nucleotides in length have similar targeting activities. Mean fluorescence intensity of AcGFP in HEK293T cells 72 hr after transfection with SOD1 reporter plasmid and RfxCas13d with SOD1-targeting crRNAs with 18, 20, 22, 24, 26, 28, 30 and 32 nucleotide protospacer sequences. Data was normalized to cells transfected with RfxCas13d and non-targeted crRNA, denoted NTG (n=3). ***P<0.001; two-tailed unpaired t test.

FIG. 6 RfxCas13d slowed the rate with which hindlimb grip strength declined in G93A-SOD1 mice. Hindlimb grip strength of G93A-SOD1 mice injected with AAV9-RfxCas13d-hSOD1 (n=15) or AAV9-RfxCas13d-NIG (n=15). Values tor each mouse were normalized to week nine values for the same mouse. Error bars depict S.E.M. ***P<0.001; linear regression analysis.

FIGS. 7A-7B RfxCas13d did not affect the expression of candidate off-target genes in vivo. (FIG. 7A) Target sequence of the SOD1 crRNA and candidate off-target sites which contained a potential pseudo-crRNA binding site with at least 15 contiguous nucleotides of homology. Mismatches in the off-target sites are denoted in orange (Zfp82 nucleotides 1-12 and 25-30; Npcd nucleotides 17-30; Cbx6 nucleotides 17-30; Trim6 nucleotides 1˜4 and 21-30; Pdcd5 nucleotides 1-5, 12, and 25-30; Cpsf6 1-12 and 28-30, Prp4fp nucleotides 1-2, 12, and 20-30). hSOD1 is SEQ ID NO:72; Zfp82 is SEQ ID NO:73; Npcd is SEQ ID NO:74; Cbx6 is SEQ ID NO:75; Trim6 is SEQ ID NO:76; Pdcd5 is SEQ ID NO:77; Cpsf6 is SEQ ID NO:78; Prp4fb is SEQ ID NO:79 (FIG. 7B) Normalized log 2 fold-change of candidate off-target transcripts from G93A-SOD1 mice injected with 2×10¹¹ particles of AAV9-RfxCas13d-hSOD1 with respect to mice injected with 2×10¹¹ particles of AAV9-RfxCas13d-NTG (n≥2). Fold-change was determined using the 2^{-ΔΔCT method. Bars illustrate means and error bars represent upper and lower error limits. **P<}0.01; two-tailed unpaired t test.

FIGS. 8A-8B Intrathecal delivery of AAV9 encoding RfxCas13d to G93A-SOD1 mice results in the efficient transduction of spinal cord astrocytes. (FIG. 8A) Representative immunofluorescence staining of the white matter of the lumbar spinal cord from end-stage G93A-SOD1 mice injected with 2×10¹¹ particles of AAV9 encoding RfxCas13d. Scale bar: 30 μm. (FIG. 8B) Percentage of dual GFAP⁺/HA⁺ astrocytes within the anterior horn of the cervical, thoracic and lumbar spinal cord from end-stage G93A-SOD1 mice injected with 2×10¹¹ particles of AAV9-RfxCas13d-NTG (n=3) or AAV9-RfxCas13d-hSOD1 (n=3). 250 cells were counted for each region for each mouse. Bars illustrate means and error bars represent S.D.

FIG. 9 Intrathecal delivery of AAV9 vector encoding RfxCas13d to G93A-SOD1 mice did not result in the efficient transduction of spinal cord microglia and neurons. Representative immunofluorescent staining of spinal cord sections four weeks after G93A-SOD1 mice were injected with 2×10¹¹ particles of AAV9 encoding RfxCas13d. Scale bars: 30 μm. Pan-neuronal markers: NeuN, neuronal nuclei marker; Tuj1, differentiated neuron marker found in the processes and cytoplasm. Microglia marker: Iba1, microglia-specific calcium binding protein.

FIGS. 10A-10C RfxCas13d-mediated silencing reduced SOD1 immunoreactive inclusions in the spinal cords of G93A-SOD1 mice. (FIG. 10A) Representative immunofluorescent staining of spinal cord sections from end-stage G93A-SOD1 mice injected with 2×10¹¹ particles of AAV9 encoding RfxCas13d. Scale bars: 30 μm. (FIG. 10B and FIG. 10C) Quantitation of surface area occupied by SOD1 immunoreactive inclusions within (FIG. 10B) the anterior horn of the lumbar, thoracic and cervical spinal cords or (FIG. 10C) the white matter of the lumbar, thoracic and cervical spinal cords from end-stage G93A-SOD1 mice injected with either AAV9-RfxCas13d-NTG or AAV9-RfxCas13d-hSOD1 (n≥4). (n≥4). An average of 50 hemisections per mouse were analyzed per mouse. Error bars indicate SD. *P<0.05, **P<0.01, ***P<0.001; two-tailed unpaired t test.

FIGS. 11A-11B RfxCas13d minimally effected the expression of housekeeping genes in HEK293T cells. (FIG. 11A) Microarray (MA) plots of RNA-seq data from HEK293T cells 72 hr after transfection with either (left) left RfxCas13d alone, (middle) RfxCas13d with HTT crRNA or (right) RfxCas13d with NTG crRNA as compared to untreated cells. Pink dots represent putative housekeeping genes (HKGs) (47). (FIG. 11B) Number of significant differentially expressed HKGs (>1.25-fold change, FDR P-value<0.01) by RNA-seq in HEK293T cells 72 hr after transfection with RfxCas13d alone, RfxCas13d with HTT crRNA, or RfxCas13d with NTG crRNA as compared to untreated cells (n=3).

FIG. 12 Nucleotide sequence of the pAAV-CAG-RfxCas13d-U6-crRNA vector constructed and used in this study. Bold, AAV ITRs. Underline, CAG promoter. Italics, Nucleoplasmin nuclear localization signal (NLS) sequence. Bold and italics, RfxCas13d. Underline and italics, 3×HA-tag. Double underline, bGH-polyadenylation signal. Double underline and italics, U6 promoter. Double underline and bold, 5′ processed direct repeats. Double underline, bold and italics, protospacer cloning site.

While the present invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention.

### DETAILED DESCRIPTION

The technology now will be described more fully hereinafter with reference to the accompanying drawings, in which some, but not all embodiments are shown. Indeed, the invention may be embodied in many different forms and should not be construed as limited to the embodiments set forth herein; rather, these embodiments are provided so that this disclosure will satisfy applicable legal requirements.

Likewise, many modifications and other embodiments of the technology described herein will come to mind to one of skill in the art to which the technology pertains having the benefit of the teachings presented in the foregoing descriptions and the associated drawings. Therefore, it is to be understood that aspects of the technology are not to be limited to the specific embodiments disclosed and that modifications and other embodiments are intended to be included. Although specific terms are employed herein, they are used in a generic and descriptive sense only and not for purposes of limitation.

Unless defined otherwise, all technical and scientific terms used herein have the same meaning as commonly understood by one of skill in the art to which the technology pertains.

Cas13 nucleases are RNA-guided RNA-targeting CRISPR effector proteins capable of mediating RNA knockdown in mammalian cells. In certain embodiments, Cas13 can be delivered to the brain and spinal cord to silence neurodegeneration-associated genes. Intrathecally delivering a vector, such as an adeno-associated virus vector, encoding a Cas13 programmed to target superoxide dismutase 1, a protein whose mutation can cause amyotrophic lateral sclerosis, reduced SOD1 mRNA and protein in the spinal cord by >50% and improved therapeutic outcomes in a mouse model of the disorder. In certain embodiments, intrastriatally delivery of Cas13 programmed to target huntingtin, a protein whose mutation is causative for Huntington's disease, led to a ˜50-60% reduction in HTT protein and its toxic aggregates in the brain. In various embodiments, Cas13 can be utilized as a versatile platform for knocking down mutant gene function in the nervous system.

### Polynucleotides

Polynucleotides can be single-stranded or double-stranded. In some embodiments, the polynucleotide is DNA. In particular embodiments, the polynucleotide is cDNA. In some embodiments, the polynucleotide is RNA. In some embodiments, the polynucleotide is included within a nucleic acid construct. In some modalities, the construct is a replicable vector. In some embodiments, the vector is selected from a plasmid, a viral vector, a phagemid, a yeast chromosomal vector and a non-episomal mammal vector.

In some embodiments, a polynucleotide is operationally linked to one or more regulatory nucleotide sequences in an expression construct.

Unless otherwise indicated, the term polynucleotide, nucleic acid molecule, or gene includes reference to the specified sequence, as well as the complementary sequence thereof, and the corresponding RNA molecule. Polynucleotides can be present as a single-stranded or double-stranded and linear or covalently circularly closed molecule. As used herein, a polynucleotide can include both naturally occurring and non-naturally occurring nucleotides.

Polynucleotides can be obtained from nucleic acid molecules present in, for example, a mammalian cell. Polynucleotides can also be synthesized in the laboratory, for example, using an automatic synthesizer. Polynucleotides can be isolated. An isolated polynucleotide can be a naturally occurring polynucleotide that is not immediately contiguous with one or both of the 5′ and 3′ flanking genomic sequences that it is naturally associated with. An isolated polynucleotide can be, for example, a recombinant DNA molecule of any length, provided that the nucleic acid molecules naturally found immediately flanking the recombinant DNA molecule in a naturally occurring genome is removed or absent. Isolated polynucleotides also include non-naturally occurring nucleic acid molecules. “Isolated polynucleotides” can be (i) amplified in vitro, for example via polymerase chain reaction (PCR), (ii) produced recombinantly by cloning, (iii) purified, for example, by cleavage and separation by gel electrophoresis, (iv) synthesized, for example, by chemical synthesis, or (vi) extracted from a sample.

### Cas13 Polypeptides

A Cas13 polypeptide can be any Cas13 polypeptide (also called “Cas13” herein) known in the art. Cas13 proteins can be directed to a specific RNA via an engineered CRISPR RNA (crRNA) guide molecule that encodes a programmable spacer sequence that mediates target engagement via RNA-RNA base complementarity. To date, several different Cas13 subtypes have been identified. Among these is the Cas13d nuclease from Ruminococcus flavefaciens XPD3002 (RfxCas13d), a class II, type VI CRISPR effector protein that possesses favorable targeting capabilities to other Cas13 orthologs, has high programmability, as it does not require a protospacer flanking sequence to bind RNA, and can fit within a single adeno-associated virus (AAV) vector particle alongside a crRNA expression cassette to enable its in vivo gene transfer. Cas13 polypeptides are RNA-targeting programmable nucleases of the Type VI CRISPR-Cas systems. Type VI CRISPR-Cas systems are RNA-targeting immune systems derived from prokaryotes. The Cas13 family comprises at least four subtypes, including Cas13a (formerly C2c2), Cas13b, Cas13c and Cas13d. Type VI-A and VI-B systems have crRNA-dependent target cleavage activity and a non-specific, collateral RNase activity that is stimulated by target recognition and cleavage. Both of these activities are mediated by the two HEPN domains contained in type VI effectors Cas13a and Cas13b.

Any suitable Cas13 can be used. Plasmids encoding Cas13 are available from Addgene (Watertown MA, Addgene.org). For example, Cas13 can be encoded by Addgene plasmid number 176303, 176304, 176305, 176306, 176307, 82381, 89898, 89906, 131012, 164862, 91905, 118963, 89901, 141320, 164857, 164858, 164859, 165078, 91902, 91925, 155366, 91924, or any other suitable plasmid.

In an aspect, a dCas13 (a catalytically dead Cas13) can be used in the methods described herein. Any suitable dCas13 can be used. Plasmids encoding dCas13 are available from Addgene. For example, dCas13 can be encoded by Addgene plasmid number 119858, 154938, 154939, 155366, 155367, 100817, 157854, or any other suitable plasmid.

Cas13a/b/c exhibit high efficiency and specificity for RNA knockdown applications in mammalian cells. Instead of a preferred PAM sequence, Cas13a requires a 3′ protospacer flanking sequence (PFS) of H, while Cas13b requires both a 3′ PFS of NAN or NNA and a 5′ PFS of D for effective RNA cleavage. Type IV-D CRISPR effectors (Cas13d), can be employed for RNA knockdown in mammalian cells. Target RNA cleavage by CRISPR/Cas13d is PFS-independent.

In some aspects a Cas13 polypeptide IS a a Cas13d protein. Cas13d polypeptides belong to the subtype VI-D system, which is a variant of type VI CRISPR-Cas systems. Cas13d systems have robust target cleavage, indiscriminate RNase activities, and the ability to process pre-crRNA. Cas13d has a small size and can be packaged into viral vectors. Cas13 polypeptides can be guided by crRNAs, which provide target specificity. Cas13 polypeptides can complex with crRNA molecules through interactions with a short hairpin sequence within them. The crRNA molecules encode a spacer sequence which guides Cas13 to its target sequence, thereby conferring targeting specificity. In addition to targeted RNase activity, Cas13 systems have indiscriminate cleavage activity after recognition and cleavage of the target transcript, leading to non-specific cleavage of any nearby single stranded RNA transcripts regardless of complementarity to the spacer. Cas13 can process its own pre-crRNAs, allowing individual short single crRNAs to be customized to target RNA.

Cas13 polypeptides can be naturally occurring or non-naturally occurring. A Cas13 polypeptide can be a mutant (e.g., have one or more amino acid insertions, deletions, or substitutions) Cas13 polypeptide. A mutant Cas13 polypeptide can have altered biological activity as compared to a naturally occurring Cas13 polypeptide, such as altered nuclease activity without substantially diminished binding affinity to RNA. A mutant Cas13 can have, for example, no nuclease activity. For example, a mutant Cas13 can be a ribonuclease that has the positively charged catalytic residues of the HEPN motifs inactivated, which eliminates programmable RNA cleavage without affecting guide RNA array processing or target RNA binding. A Cas13 polypeptide can be a Cas13d polypeptide. A Cas13d polypeptide can be from any suitable bacterial species, for example, Ruminococcus sp., Ruminoccocus flavefaciens, Ruminoccocus albus, and Eubacterium siraeum. In some aspects, the Cas13d polypeptide is derived from Ruminococcus flavefaciens strain XPD3002 (e.g., CasRx or RfxCas13d). In an aspect, a Cas13d polypeptide is a catalytically inactive version of CasRx (e.g. dCasRx). An exemplary sequence of CasRx (NLS-RfxCas13d-NLS) can be found at Plasmid #109049 (pXR001: EF1a-CasRx-2A-EGFP, Addgene). In an aspect, a nucleotide sequence encoding a Cas13 polypeptide can be at least about 80% identical (e.g. at least 80%, 85%, 90%, 92%, 94%, 96%, 98%, or 99% identical) to the sequence of RfxCas13d.

In some aspects, a polynucleotide can comprise a sequence encoding a Cas13 protein and one or more (1, 2, 3, 4, 5, or more) localization signals. A localization signal tag a protein for transportation to a particular location in a cell. In an aspect a localization signal is a nuclear localization signal (NLS), which can be an amino acid sequence that tags a protein for import into the cell nucleus by nuclear transport. Localization signals can be operably linked to the sequence encoding a Cas13 protein. For example, the sequence encoding Cas13 can comprise two nuclear localization signals such that a Cas13 polypeptide is expressed that is fused to N- and C-terminal nuclear localization signals. An NLS can be, for example, SV40 large T antigen NLS (PKKKRRV (SEQ ID NO:1)) or nucleoplasmin NLS (KRPAATKKAGQAKKKK (SEQ ID NO:2)). Other NLSs are described in, for example, Konermann et al., Cell 173:665-676, 2018; Cokol et al., EMBO Rep. 1(5):411-415 (2000); Freitas & Cunha, Curr Genomics 10(8): 550-557 (2009).

A polynucleotide encoding a Cas13 polypeptide can be operably linked to a promoter such as ubiquitous promoters (e.g., ubiquitin promoter), tissue-specific promoters, inducible promoters, and constitutive promoters.

A polynucleotide encoding a Cas13 polypeptide can be operably linked to a sequence that encodes one or more reporter polynucleotides. Reporter polynucleotides include, for example, fluorescent reporters.

crRNA Molecules and Cas13 Repeat Arrays

crRNA molecules can comprise a Cas13-specific direct repeat (DR) region, which forms a hairpin structure when transcribed. A Cas13-specific direct repeat (DR) region can be specific for Cas13a, Cas13b, Cas13c, or Cas13d. The hairpin structure enables Cas13 to bind to the crRNA, effectively forming a Cas13-crRNA complex. crRNA molecules can also comprise a protospacer region, which has homology to a target nucleic acid molecule (e.g., an SOD1 or HTT gene or promoter).

Cas13 repeat array polynucleotides can encode one or more crRNAs and one or more Cas13-specific direct repeats,

A Cas13 repeat array polynucleotide can comprise one or more (e.g., 1, 2, 3, 4, 5, 6, 7, or more) crRNAs and one or more (e.g., 1, 2, 3, 4, 5, 6, 7, or more) Cas13-specific direct repeats. The crRNAs can have homology to the same or different target sequences in the same target RNA or can bind to different target RNAs. The crRNAs can be designed to have homology to any sequence in a target RNA. In instances where two or more crRNAs are included in a Cas13 repeat array polynucleotide, the crRNAs can have the same or different length. The crRNAs can comprise about 20 to 40 nucleotides (e.g., about 20, 25, 26, 27, 28, 29, 30, 35, or 40 nucleotides).

A Cas13d repeat array polynucleotide can comprise about 1 or more (e.g., 1, 2, 3, 4, 5, 6, 7, 8 or more) Cas13-specific direct repeats. A direct repeat can be a repetitive sequence within a CRISPR locus that are interspersed by short spacers. A direct repeat sequence can have homology to a trans-activating CRISPR RNA. A direct repeat facilitates formation of a crRNA: tracrRNA duplex. The sequence and secondary structure of Cas13-specific direct repeats can be dependent on the specific Cas13. For instance, Cas13d from different species can have different direct repeat sequences and/or secondary structures. Exemplary direct repeat sequences for Cas13d can be found at e.g., Konnerman et al., Cell 1/3:665-6/6 (2018). The Cas13 specific direct repeat sequences can be about 30 to about 40 nucleotides in length (e.g., about 30, 31, 32, 33, 34, 35, 36, 37, 38, 39, or 40) nucleotides. A Cas13 specific direct repeat can form a hairpin structure that interacts with a Cas13 polypeptide to form a complex.

A Cas13d-specific direct repeat can be, for example, CAAGUAAACCCCUACCAACUGGUCGGGGUUUGAAAC (SEQ ID NO:3) (can be used as a DNA sequence in a vector) or GAAACACCGAACCCCTACCAACTGGTCGGGGTTTG (SEQ ID NO:90) (DNA to be expressed in cells from a transfected vector) or at least 80% identical (e.g. at least 82%, 84%, 86%, 88%, 90%, 92%, 94%, 96%, 98%, 99% identical) to SEQ ID NO:3 or 90. crRNAs can be arranged in tandem and interspersed by direct repeats. For example, a crRNAs can be positioned between two direct repeats.

Provided herein are nucleic acid molecules comprising Cas13-specific repeat arrays or crRNA molecules. Also provided herein are vectors comprising nucleic acid molecules comprising Cas13-specific repeat arrays or crRNA. Nucleic acid molecules encoding Cas13-specific repeat arrays or crRNA can be operably linked to one or more promoters. Any suitable promoter can be used including, for example, a polymerase III promoter, such as a polymerase-3 U6 (U6:3) promoter.

In some aspects a crRNA has homology to a SOD1 gene or an HTT gene, for example Exon 1 of an HTT gene.

The target nucleic acid sequence (the portion of the gene targeted by a crRNA and a Cas13 nuclease) of a SOD1 gene or an HTT gene can comprise about 20 nucleotides. The target nucleic acid can comprise less than about 20 nucleotides. The target nucleic acid can comprise more than 20 nucleotides. The target nucleic acid can comprise at least about 5, 10, 15, 16, 17, 18, 19, 20, 21, 22, 23, 24, 25, 30 or more nucleotides. The target nucleic acid can comprise at most: 5, 10, 15, 16, 17, 18, 19, 20, 21, 22, 23, 24, 25, 30 or more nucleotides.

A crRNA can be about 26, 27, 28, 29, 30, 31, 32, 33, 34, 35, 36, 37, 38, 39 or more nucleotides in length. The crRNA sequence that hybridizes to the target nucleic acid can have a length of at least about 6 nucleotides (nt). The crRNA sequence can be at least about 6 nt, at least about 10 nt, at least about 15 nt, at least about 18 nt, at least about 19 nt, at least about 20 nt, at least about 25 nt, at least about 30 nt, at least about 35 nt or at least about 40 nt, from about 6 nt to about 80 nt, from about 6 nt to about 50 nt, from about 6 nt to about 45 nt, from about 6 nt to about 40 nt, from about 6 nt to about 35 nt, from about 6 nt to about 30 nt, from about 6 nt to about 25 nt, from about 6 nt to about 20 nt, from about 6 nt to about 19 nt, from about 10 nt to about 50 nt, from about 10 nt to about 45 nt, from about 10 nt to about 40 nt, from about 10 nt to about 35 nt, from about 10 nt to about 30 nt, from about 10 nt to about 25 nt, from about 10 nt to about 20 nt, from about 10 nt to about 19 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some examples, the crRNA can comprise 30 nucleotides. In some examples, the spacer sequence can comprise 28 nucleotides. In some examples, the spacer sequence can comprise 29 nucleotides. In some examples, the spacer sequence can comprise 31 nucleotides. In some examples, the spacer sequence can comprise 32 nucleotides.

In some examples, the percent complementarity between the crRNA sequence and the target nucleic acid is at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. In some examples, the percent complementarity between the crRNA and the target nucleic acid is at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%. In some examples, the percent complementarity between the crRNA and the target nucleic acid is 100% over the six contiguous 5′-most nucleotides of the target sequence of the complementary strand of the target nucleic acid. The percent complementarity between the crRNA and the target nucleic acid can be at least 60% over about 20 contiguous nucleotides. The length of the spacer sequence and the target nucleic acid can differ by 1 to 6 nucleotides, which may be thought of as a bulge or bulges.

The crRNA sequence can be designed or chosen using a computer program. The computer program can use variables, such as predicted melting temperature, secondary structure formation, predicted annealing temperature, sequence identity, genomic context, chromatin accessibility, % GC, frequency of genomic occurrence (e.g., of sequences that are identical or are similar but vary in one or more spots as a result of mismatch, insertion or deletion), methylation status, presence of SNPs, and the like.

A Cas13-specific repeat array or crRNA can be operably linked to nucleic acid molecules that encode one or more reporter genes, such as a fluorescent reporter gene.

A purified crRNA or Cas13-specific repeat array is a polynucleotide preparation that is substantially free of cellular material, other types of polynucleotides, chemical precursors, chemicals used in synthesis of the polynucleotide, or combinations thereof. A polynucleotide preparation that is substantially free of chemical precursors, chemicals used in synthesis, etc. of the polynucleotide has less than about 30%, 20%, 10%, 5%, 1% or more of other polynucleotides, chemical precursors, and/or other chemicals used in synthesis. Therefore, a purified polynucleotide (e.g., a crRNA or Cas13-specific repeat array) is about 70%, 80%, 90%, 95%, 99% or more pure.

### Vectors

Vectors such as plasmid or viral vectors (e.g. viral vectors) can comprise nucleic acid molecules encoding a Cas13 polypeptide (e.g. any Cas13 polypeptides described herein) and/or a nucleic acid molecule encoding a Cas13-specific repeat array or crRNA (e.g. any Cas13-specific repeat array or crRNA described herein). Any suitable vectors can be used. A vector can comprise, for example, any genetic element including, without limitation, naked DNA, a phage, transposon, cosmid, episome, plasmid, bacteria, or a virus, which expresses, or causes to be expressed, a desired nucleic acid construct (e.g., a crRNA and/or a Cas13 nuclease). Thus, in one embodiment, the vector is a non-pathogenic virus. A vector can be, for example, a non-replicating virus. In one aspect, a viral vector can be a retroviral vector, such as a lentiviral vector. A viral vector can be, e.g., an adeno-associated viral vector (AAV).

AAV is a replication-deficient parvovirus, the single-stranded DNA genome of which is about 4.7 kb in length, including two 145-nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_001862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAV rh.74 genome is provided in U.S. Pat. No. 9,434,928, incorporated herein by reference in its entirety. U.S. Pat. No. 9,434,928 also provides the sequences of the capsid proteins and a self-complementary genome. In one aspect, an AAV genome is a self-complementary genome. Cis-acting sequences directing viral DNA replication (rep), encapsidation/packaging, and host cell chromosome integration are contained within AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of tour rep proteins (rep/8, rep 68, rep 52, and rep 40) from the rep gene. Rep proteins possess multiple enzymatic properties that are ultimately responsible for replicating the viral genome.

A cap gene can be expressed from the p40 promoter and encodes the three capsid proteins, VPI, VP2, and VP3. Alternative splicing and non-consensus translational start sites are responsible for the production of the three related capsid proteins. More specifically, after the single mRNA from which each of the VP1, VP2 and VP3 proteins are translated is transcribed, it can be spliced in two different manners: either a longer or shorter intron can be excised, resulting in the formation of two pools of mRNAs: a 2.3 kb- and a 2.6 kb-long mRNA pool. The longer intron is often preferred and thus the 2.3-kb-long mRNA can be called the major splice variant. This form lacks the first AUG codon, from which the synthesis of VP1 protein starts, resulting in a reduced overall level of VP1 protein synthesis. The first AUG codon that remains in the major splice variant is the initiation codon for the VP3 protein. However, upstream of that codon in the same open reading frame lies an ACG sequence (encoding threonine) which is surrounded by an optimal Kozak (translation initiation) context. This contributes to a low level of synthesis of the VP2 protein, which is actually the VP3 protein with additional N terminal residues, as is VP1, as described in Becerra S P et al., (December 1985). “Direct mapping of adeno-associated virus capsid proteins B and C: a possible ACG initiation codon”. Proceedings of the National Academy of Sciences of the United States of America. 82 (23): 7919-23, Cassinotti P et al., (November 1988). “Organization of the adeno-associated virus (AAV) capsid gene: mapping of a minor spliced mRNA coding for virus capsid protein 1”. Virology. 167 (1): 176-84, Muralidhar S et al., (January 1994). “Site-directed mutagenesis of adeno-associated virus type 2 structural protein initiation codons: effects on regulation of synthesis and biological activity” Journal of Virology. 68 (1): 170-6, and Trempe J P, Carter B J (September 1988). “Alternate mRNA splicing is required for synthesis of adeno-associated virus VP1 capsid protein”. Journal of Virology. 62 (9): 3356-63, each of which is herein incorporated by reference. A single consensus polyA site is located at map position 95 of the AAV genome. The life cycle and genetics of AAV are reviewed in Muzyczka, Current Topics in Microbiology and Immunology, 158: 97-129 (1992).

Each VP1 protein contains a VP1 portion, a VP2 portion and a VP3 portion. The VP1 portion is the N-terminal portion of the VP1 protein that is unique to the VP1 protein. The VP2 portion is the amino acid sequence present within the VP1 protein that is also found in the N-terminal portion of the VP2 protein. The VP3 portion and the VP3 protein have the same sequence. The VP3 portion is the C-terminal portion of the VP1 protein that is shared with the VP1 and VP2 proteins.

The VP3 protein can be further divided into discrete variable surface regions I-IX (VR-I-IX). Each of the variable surface regions (VRs) can comprise or contain specific amino acid sequences that either alone or in combination with the specific amino acid sequences of each of the other VRs can confer unique infection phenotypes (e.g., decreased antigenicity, improved transduction and/or tissue-specific tropism relative to other AAV serotypes) to a particular serotype as described in DiMatta et al., “Structural Insight into the Unique Properties of Adeno-Associated Virus Serotype 9” J. Virol., Vol. 86 (12): 6947-6958, June 2012, the contents of which are incorporated herein by reference.

AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) can be replaced with foreign DNA to generate AAV vectors. The rep and cap proteins can be provided in trans. Another significant feature of AAV is that it is an extremely stable and hearty virus. It easily withstands the conditions used to inactivate adenovirus (56° to 65° C. for several hours), making cold preservation of AAV less critical. AAV can be lyophilized. Advantageously, AAV-infected cells are not resistant to superinfection.

AAV DNA in the rAAV genomes may be from any AAV serotype for which a recombinant virus can be derived including, but not limited to, AAV serotypes AAV1, AAV2, AAV3, AAV4, AAV5, AAV6, AAV7, AAV8, AAV9, AAV10, AAV11, AAV12, AAV13, AAVPHP.B, AAVrh74, and AAVrh.10. Production of pseudotyped rAAV is disclosed in, for example, WO2001083692. Other types of rAAV variants, for example rAAV with capsid mutations, are also contemplated. See, e.g., Marsic et al., Molecular Therapy, 22(11): 1900-1909 (2014).

A recombinant adeno-associated virus (rAAV) vector can comprise in 5′ to 3′ direction a first AAV inverted terminal repeat (ITR) sequence, a promoter sequence, a nucleic acid molecule encoding a Cas13 polypeptide, a polyA sequence, a crRNA sequence having homology to a superoxide dismutase 1 (SOD1) gene or a huntingtin (HTT) gene, and a second AAV ITR sequence.

In some aspects, a promoter as described herein can be used drive expression of a crRNA sequence. Therefore, a promoter, such as a type III RNA polymerase III promoter (e.g., U6 promoter) can be present in a vector in association with crRNA sequences. For example, a promoter can be present 5′ to a crRNA sequence or 5′ to a series of crRNA sequences, where more than one crRNA sequence is used. In some embodiments, where more than one crRNA sequences are present, a promoter can be present in association with each and every crRNA, e.g., 5′ to each crRNA sequence.

Other elements can include, for example, one or more nuclear localization signals (NLSs), one or more promoters, and one or more tags.

### Nuclear Localization Signals

A nuclear localization signal (NLS) can drive a protein to the cell nucleus through the Nuclear Pore Complex and can be used to improve delivery efficiency. An NLS can generally comprise one or more short sequences of positively charged amino acids such as lysines or arginines. Examples of NLSs include SV40 large T antigen (PKKKRKV; SEQ ID NO:80), nucleoplasmin (KR[PAATKKAGQA]KKKK; (SEQ ID NO:81), p54 (RIRKKLR; SEQ ID NO:82), SOX9 (PRRRK (SEQ ID NO:83), NS5A PPRKKRTVV (SEQ ID NO:84), SPKKKRKVEAS (SEQ ID NO:85) or GPKKKRKVAAA (SV40 large T antigen NLS SEQ ID NO:86).

One or more NLSs can occur 5′ to the nucleic acid molecule encoding the Cas13 polypeptide, 3′ to the nucleic acid molecule encoding the Cas13 polypeptide, or both.

### Promoters and Enhancers

A vector can comprise a promoter and/or an enhancer. A promoter or promoter sequence controls the initiation and rate of transcription of a coding sequence, such as a gene or a transgene. Promoters can be, for example, constitutive, inducible, repressible, or tissue-specific. Promoters can contain genetic elements for binding of regulatory proteins and molecules such as RNA polymerase and transcription factors. In some embodiments, the promoter is a viral promoter, e.g., a CMV, HIV, adenovirus, or AAV promoter. Any suitable promoter can be used, such as a cytomegalovirus early enhancer/chicken β-actin (CAG) promoter, Rous sarcoma virus (RSV) LTR promoter (optionally with the RSV enhancer), a cytomegalovirus (CMV) promoter, an SV40 promoter, a dihydrofolate reductase promoter, a β-actin promoter, a phosphoglycerol kinase (PGK) promoter, a U6 promoter (e.g., GAGGGCCTATTTCCCATGATTCCTTCATATTTGCATATACGATACAAGGCTGTTAGAGAGATAATTGG AATTAATTTGACTGTAAACACAAAGATATTAGTACAAAATACGTGACGTAGAAAGTAATAATTTCTTG GGTAGTTTGCAGTTTTAAAATTATGTTTTAAAATGGACTATCATATGCTTACCGTAACTTGAAAGTAT TTCGATTTCTTGGCTTTATATATCTTGTGGAAAGGAC (SEQ ID NO:91), an H1 promoter, a ubiquitous chicken β-actin hybrid (CBh) promoter, EFla promoter, Ubc promoter, human β-actin promoter, TRE promoter, Ac5 promoter, polyhedrin promoter, CaMKIIa promoter, Gal1 promoter, TEF1 promoter, GDS promoter, ADH1 promoter, Ubi promoter, α-1-antitrypsin (hAAT) promoter, or small nuclear RNA (U1a or U1b) promoter.

In some aspects, the promoter is used together with at least one enhancer to increase the transcription efficiency. Non-limiting examples of enhancers include an interstitial retinoid-binding protein (IRBP) enhancer, an RSV enhancer or a CMV enhancer. An enhancer can increase the expression of a target sequence. A promoter/enhancer is a polynucleotide that contains sequences capable of providing both promoter and enhancer functions. For example, the long terminal repeats of retroviruses contain both promoter and enhancer functions. An enhancer and/or promoter can be endogenous or exogenous (i.e., heterologous) An endogenous enhancer/promoter is naturally linked with a particular gene or nucleic acid sequence in the genome. An exogenous enhancer/promoter added or linked to a gene or nucleic acid sequence by genetic manipulation (i.e., molecular biological techniques) such that transcription of that gene is directed by the linked enhancer/promoter. Examples of enhancer/promoters include a PDE promoter plus IRBP enhancer or a CMV enhancer plus U1a promoter. Enhancers can operate from a distance and irrespective of their orientation relative to the location of an endogenous or heterologous promoter. Therefore, an enhancer operating at a distance from a promoter is operably linked to that promoter irrespective of its location in the vector or its orientation relative to the location of the promoter.

Operably linked refers to the expression of a gene (i.e., a transgene) that is under the control of a promoter. A promoter can be positioned 5′ (upstream) or 3′ (downstream) of a gene under its control. The distance between a promoter and a gene can be approximately the same as the distance between that promoter and the gene it controls in the gene from which the promoter is derived. Variation in the distance between a promoter and a gene can be accommodated without loss of promoter function.

### Tags

A vector, such as a recombinant rAAV vector, can further comprise one or more tags. For example, a human influenza hemagglutinin (HA) epitope tags can be present. An HA-tag can comprise TAC CCA TAC GAT GTT CCA GAT TAC GCT (SEQ ID NO:87) or TAT CCA TAT GAT GTT CCA GAT TAT GCT (SEQ ID NO:88). An HA epitope tag amino acid sequence can be YPYDVPDYA (SEQ ID NO:89). Other suitable epitope tags can be used such as V5 epitope tag, a FLAG tag, a tandem FLAG-tag, a triple FLAG tag, a tandem HA tag, a triple HA tag (3×HA), a sextuple Histidine tag (6×HIS), biotin, c-MYC, a Glutathione-S-transferase (GST) tag, a Strep-tag, a Strep-tag II, a S-tag, a natural histidine affinity tag (HAT), a Calmodulin-binding peptide (CBP) tag, a Streptavidin-binding peptide (SBP) tag, a Chitin-binding domain, a Maltose-binding protein (MBP), or derivatives thereof

### PolyA

A vector, such as a recombinant rAAV vector, can comprise a polyadenylation (polyA) sequence. Any polyA sequence known in the art can be used. Non-limiting examples of polyA sequences include, but are not limited to, a bovine growth hormone (BGH) polyA sequence, a retinol dehydrogenase 1 (RDH1) polyA sequence, an SV40 polyA sequence, a SPA49 polyA sequence, a sNRP-TK65 polyA sequence, a sNRP polyA sequence, or a TK65 polyA sequence.

### Bacterial Plasmids

In some aspects, rAAV vectors can be contained within a bacterial plasmid to allow for propagation of the rAAV vector in vitro. Therefore, provided herein are bacterial plasmids comprising any of the rAAV vectors described herein. A bacterial plasmid can further comprise an origin of replication sequence, an antibiotic resistance gene, a prokaryotic promoter, or a combination thereof.

### Target RNA and Methods of Modifying a Target RNA in a Cell

Target RNA can be any SOD1 or HTT RNA molecules endogenous or exogenous to a eukaryotic cell and can be protein-coding (e.g., SOD1 mRNA or HTT mRNA) or non-protein-coding (e.g., an SOD1 or HTT promoter or enhancer). A Cas13-specific repeat array can include one or more crRNAs (e.g., 1, 2, 3, 4, 5, 6, 7, 8, or more) that can specifically hybridize with the same target RNA or at least two different target RNAs (e.g., 2, 3, 4, 5, 6, 7, 8, or more).

Methods are provided for modifying a target RNA in a cell. The methods can include introducing a nucleic acid molecule encoding a Cas13 polypeptide (e.g., any of the Cas13 polypeptides described herein) and a crRNA or Cas13-specific repeat array (e.g., any of the crRNA molecules or Cas13-specific repeat arrays_described herein) into the cell. Nucleic acid molecules encoding a Cas13protein, crRNA, or Cas13-specific repeat arrays can be introduced into the cell in the same nucleic acid molecule or in different nucleic acid molecules.

A method can comprise contacting, via, e.g., transfection, the cell with (a) a nucleic acid molecule encoding a Cas13 polypeptide, and (b) a crRNA or a Cas13-specific repeat array comprising one or more crRNAs and one or more Cas13-specific direct repeats, wherein the one or more crRNAs can specifically hybridize with the target RNA. In some aspects a nucleic acid molecule encoding a Cas13 polypeptide can be introduced by a first vector and a Cas13-specific repeat array or a crRNA can be introduced by a second vector.

### Pharmaceutical Compositions

In an aspect, pharmaceutical compositions comprising any of the isolated polynucleotides, vectors, rAAV vectors, and/or rAAV viral vectors described herein are provided.

A pharmaceutical composition can be formulated by any suitable methods, which include but are not limited to contacting the active ingredients (e.g., viral particles or recombinant vectors) with an excipient and/or additive or other accessory ingredient, dividing or packaging the product to a dose unit. Vectors such as viral particles can be formulated with desirable features, e.g., increased stability, increased cell transfection, sustained or delayed release, biodistributions or tropisms, modulated or enhanced translation of encoded protein in vivo, and the release profile of encoded protein in vivo.

Therefore, pharmaceutical compositions can comprise, for example, saline, lipidoids, liposomes, lipid nanoparticles, polymers, lipoplexes, core-shell nanoparticles, peptides, proteins, cells transfected with viral vectors (e.g., for transplantation into a subject), nanoparticle mimics, or combinations thereof. A pharmaceutical composition can be formulated as a nanoparticle such as a self-assembled nucleic acid nanoparticle.

A pharmaceutical composition can be prepared, packaged, and/or provided in bulk, as a single unit dose, and/or as a plurality of single unit doses. The amount of the active ingredient is generally equal to the dosage of the active ingredient which would be administered to a subject and/or a convenient fraction of such a dosage such as, for example, one-half or one-third of such a dosage. Formulations can include one or more excipients and/or additives, each in an amount that together increases the stability of the viral vector, increases cell transfection or transduction by the viral vector, increases the expression of viral vector encoded protein, and/or alters the release profile of viral vector encoded proteins. In some aspects, a pharmaceutical composition can comprise an excipient and/or additives. Non limiting examples of excipients and/or additives include solvents, dispersion media, diluents, or other liquid vehicles, dispersion or suspension aids, surface active agents, isotonic agents, thickening or emulsifying agents, preservatives, or combinations thereof.

A pharmaceutical composition can comprise a cryoprotectant, which is an agent that can reduce or eliminate damage to a substance during freezing. Non-limiting examples of cryoprotectants include sucrose, trehalose, lactose, glycerol, dextrose, raffinose, and/or mannitol.

A pharmaceutically acceptable carrier is any standard pharmaceutical carrier, such as a phosphate buffered saline solution, water, and emulsions, such as an oil/water or water/oil emulsion, and various types of wetting agents. A pharmaceutical composition also can include stabilizers and preservatives. For examples of carriers, stabilizers, and adjuvants, see Martin (1975) Remington's Pharm. Sci., 15th Ed. (Mack Publ. Co., Easton).

### Methods of Treatment

Provided herein is the use of a composition or pharmaceutical composition for the treatment of a disease or disorder in a cell, tissue, organ, animal, or subject, by administering or contacting the cell, tissue, organ, animal, or subject with a therapeutic effective amount of the composition or pharmaceutical composition. In one aspect, the subject is a mammal such as a human. A subject is not limited to a specific species and includes non-human animals subject to diagnosis or treatment and those subject to intections or animal models, including, without limitation, simian, murine, rat, canine, or leporid species, as well as other livestock, sport animals, or pets.

An aspect provides methods of preventing or treating a disorder by administering to a subject at least one therapeutically effective amount of any one of the pharmaceutical compositions disclosed herein. In some aspects the disease can be a genetic disorder involving a SOD1 or HTT gene.

In some aspects, the disclosure provides methods of preventing or treating diseases caused by a mutated or defective SOD1 gene such as amyotrophic lateral sclerosis (ALS), familial amyotrophic lateral sclerosis, and Parkinson's disease. A SOD1 target gene can have any mutation, for example, an A4V, H46R, G93A or other mutation.

In some aspects, the disclosure provides methods of preventing or treating diseases caused by a mutated or defective HTT gene such as Huntington's disease. Htt is variable in its structure, as the many polymorphisms of the gene can lead to variable numbers of glutamine residues present in the protein. In its wild-type form Htt contains 6-35 glutamine residues. However, in individuals affected by Huntington's disease it contains more than 36 glutamine residues (the highest reported repeat length is about 250). Therefore, a target HTT gene can encode a Htt protein comprising more than 36 glutamine residues (e.g., about 36, 40, 45, 50, 60, 70, 80, 90, 100, 150, 200, 250 or more).

A subject to be treated using the methods, compositions, pharmaceutical compositions, rAAV vectors or rAAV viral vectors of the present disclosure can have any of the diseases and/or symptoms described herein. In some aspects, methods of treatment can decrease muscle atrophy, provide for improved neuromuscular function, slow the overall progression of the disease or disorder, and increase survival. In an aspect, the expression of mutant SOD1 in astrocytes, in cervical regions, in thoracic regions, in the lumbar region, in the whole spinal cord, is reduced by about 10, 20, 30, 40, 50, 60, 70, 80% or more. In an aspect, 10, 20, 30, 40, 50, 60, 70, 80% or more fewer SOD1 immunoreactive inclusions can be present in the spinal cord after treatment. In an aspect, methods of treatment result in improved rotarod and grip strength.

In some aspects, a subject can be less than 0.5 years of age, or less than 1 year of age, or less than 1.5 years of age, or less than 2 years of age, or at less than 2.5 years of age, or less than 3 years of age, or less than 3.5 years of age, or less than 3.5 years of age, or less than 4 years of age, or less than 4.5 years of age, or less than 5 years of age, or less than 5.5 years of age, or less than 6 years of age, or less than 6.5 years of age, or less than 7 years of age, or less than 7.5 years of age, or less than 8 years of age, or less than 8.5 years of age, or less than 9 years of age, or less than 9.5 years of age, or less than 10 years of age. In some aspects the subject can be less than 11 years of age, less than 12 years of age, less than 13 years of age, less than 14 years of age, less than 15 years of age, less than 20 years of age, less than 30 years of age, less than 40 years of age, less than 50 years of age, less than 60 years of age, less than 70 years of age, less than 80 years of age, less than 90 years of age, less than 100 years of age, less than 110 years of age, or less than 120 years of age. In some aspects, a subject can be less than 0.5 years of age. In some aspects, a subject can be less than 4 years of age. In some aspects, a subject can be less than 10 years of age.

The methods of treatment and prevention disclosed herein can be combined with appropriate diagnostic techniques to identify and select patients for the therapy or prevention.

The disclosure provides methods of reducing a level of mutant HTT mRNA or mutant SOD1 mRNA in a cell or patient by contacting the host cell or patient with any one of the rAAV viral vectors disclosed herein. In some aspects the amount of mutant mRNA is reduced by about 1, 5, 10, 20, 30, 40, 50, 60, 70, 80, 90% or more as compared to untreated cells or patients. In some aspects, the host cell is in vitro, in vivo, or ex vivo. In some aspects, the host cell is derived from a subject. In some aspects, the subject suffers from a disorder caused by a mutant HTT gene or mutant SOD1 gene.

In some embodiments a subject can also be administered a prophylactic immunosuppressant treatment regimen in addition to being administered an rAAV vector. An immunosuppressant treatment regimen can comprise administering at least one immunosuppressive therapeutic. Non limiting examples of immunosuppressive therapeutics include, Sirolimus (rapamycin), acetaminophen, diphenhydramine, IV methylprednisolone, prednisone, or any combination thereof. An immunosuppressive therapeutic can be administered prior to the day of administration of the rAAV vector and/or rAAV viral vector, on the same day as the administration of the rAAV vector and/or rAAV viral vector, or any day following the administration of the rAAV vector and/or rAAV viral vector.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

An effective amount means a quantity sufficient to achieve a desired effect. In the context of therapeutic or prophylactic applications, an effective amount will depend on the type and severity of the condition at issue and the characteristics of the individual subject, such as general health, age, sex, body weight, and tolerance to pharmaceutical compositions. In some aspects, the effective amount of an rAAV viral vector is the amount sufficient to result in a decrease in the amount of mutant mRNA from mutant HTT genes or mutant SOD1 genes. A skilled artisan will be able to determine appropriate amounts depending on these and other factors.

In some aspects, the effective amount will depend on the size and nature of the application in question. It will also depend on the nature and sensitivity of the target subject and the methods in use. The skilled artisan will be able to determine the effective amount based on these and other considerations. The effective amount can comprise one or more administrations of a composition (e.g., 1, 2, 3, 4, 5, 10 or more) depending on the embodiment.

As used herein, the term “administer” or “administration” is the delivery of a substance to a subject such as an animal or human. Administration can be effected in one dose, continuously, or intermittently throughout the course of treatment. Methods of determining the most effective means and dosage of administration can vary with the composition used for therapy, the purpose of the therapy, as well as the age, health, or gender of the subject being treated. Single or multiple administrations can be carried out with the dose level and pattern being selected by the treating physician or in the case of pets and other animals, treating veterinarian.

Single or multiple administrations can be carried out with the dose level and pattern being selected by the treating physician. It is noted that dosage can be impacted by the route of administration. Non-limiting examples of dosages can be as low as 10⁸ vector genomes to as much as 10¹⁷ vector genomes per administration.

In some aspects of the methods described herein, the number of vector particles (e.g., rAAV viral vectors) administered to the subject ranges from about 10⁸ to about 10¹⁷. In some aspects, about 10⁹ to about 10¹². In some aspects, about 10¹⁰ to about 10¹², about 10¹¹ to about 10¹³, about 10¹¹ to about 10¹², about 10¹¹ to about 10¹⁴, about 10¹² to about 10¹⁶, about 10¹³ to about 10¹⁶, about 10¹⁴ to about 10¹⁵, about 5×10¹¹ to about 5×10¹², or about 10¹² to about 10¹³ viral particles are administered to the subject.

In some aspects the number of viral particles (e.g., rAAV viral vectors) administered to the subject can be at least about 10¹⁰, or at least about 10¹¹, or at least about 10¹², or at least about 10¹³, or at least about 10¹⁴, or at least about 10¹⁵, or at least about 10¹⁶, or at least about 10¹⁷ viral particles.

In some aspects of the methods, the number of viral particles (e.g., rAAV viral vectors) administered to the subject can depend on the age of the subject. In non-limiting examples, a subject that is 7 years of age or older can be administered about 10×10¹⁴ viral particles, a subject that is about 4 years of age to about 7 years of age can be administered about 10×10¹⁴ viral particles, a subject that is about 3 years of age to about 4 years of age can be administered about 9×10¹⁴ viral particles, a subject that is about 2 years of age to about 3 years of age can be about 8.2×10¹⁴ viral particles, a subject that is about 1 year of age to about 2 years of age can be administered about 7.3×10¹⁴ viral particles, a subject that is about 0.5 years of age to about 1 year of age can be administered about 4×10¹⁴ viral particles, or a subject that is less than 0.5 years of age can be administered 3×10¹⁴ viral particles.

In some aspects, the amount of viral particles in a composition, pharmaceutical composition, or the amount of viral particles administered to a patient can calculated based on the percentage of viral particles that are predicted to contain viral genomes.

In some aspects, rAAV viral vectors can be introduced to the subject intravenously, intrathecally, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally, intra-arterial, intracardiac, subventricular, epidural, intracerebral, intracerebroventricular, sub-retinal, intravitreal, intraarticular, intraperitoneal, intrauterine, or any combination thereof. In some aspects, the rAAV vectors, rAAV viral vectors, compositions, or pharmaceutical compositions of this disclosure are parenterally administered by injection, infusion, or implantation. In some aspects, the vectors, e.g., viral particles, are delivered to a desired target tissue, e.g., to the brain, spinal cord, or CNS, as non-limiting examples. In some aspects, delivery of vectors such as viral particles is systemic. The intracisternal route of administration involves administration of a drug directly into the cerebrospinal fluid of the brain ventricles. It could be performed by direct injection into the cisterna magna or via a permanently positioned tube. In some aspects, vectors, such as rAAV viral vectors are administered intrathecally.

In some aspects, the vectors, such as rAAV viral vectors, show enhanced tropism for brain and cervical spine. In some aspects, vectors, such as rAAV viral vectors of the disclosure can cross the blood-brain-barrier (BBB).

### Transgenic Organisms

Provided herein are transgenic organisms, which can include a non-human animal in where one or more of the cells of the organism includes a transgene. The organism can be a vertebrate or an invertebrate, such as an arthropod. A transgenic organism can have one or more recombinant nucleic acid molecules stably integrated into the genome of the organism, wherein the recombinant nucleic acid molecule encodes a Cas13 polypeptide (e.g. any of the Cas13 polypeptides described herein). In some aspects a transgenic organism can have two or more recombinant nucleic acid molecules stably integrated into the genome of the organism, comprising at least a first recombinant nucleic acid molecule that encodes a Cas13 polypeptide, and a second recombinant nucleic acid molecule that comprises a sequence that encodes a crRNA or a Cas13-specific repeat array.

A transgenic animal comprising a recombinant nucleic acid molecule encoding a Cas13 polypeptide can be identified based upon the presence of the nucleic acid sequence in its genome and/or expression of Cas13 in tissues or cells of the animal. A transgenic animal comprising a recombinant nucleic acid molecule encoding a crRNA can be identified based upon the presence of the nucleic acid sequence in its genome. A transgenic animal can be used to breed additional animals carrying the one or more transgenes. A transgenic animal can be heterozygous or homozygous for the one or more transgenes.

Methods for making transgenic animals include, for example, pronuclear microinjection retrovirus mediated gene transfer into germ lines, gene targeting into embryonic stem cells, electroporation of embryos, and in vitro transformation of somatic cells, such as cumulus or mammary cells, followed by nuclear transplantation.

Also provided herein are populations of cells isolated from a transgenic organism, as well as primary or cultured host cells, e.g., isolated host cells, engineered to include a nucleic acid molecule sequence that encodes one or more Cas13 proteins and/or crRNAs. The cells can be isolated from any of the transgenic animals described above. Also provided herein are methods of introducing transgenes described herein into a host cell (e.g., primary cells or cultured cells) by, for example, viral delivery.

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### EXAMPLES

### Example 1

In certain embodiments, we demonstrate that RfxCas13d can be efficiently delivered to the mouse spinal cord and brain to silence the dominant mutant genes responsible for an inherited form of ALS and HD. In certain embodiments, we show that intrathecally administering an AAV vector encoding RfxCas13d with a crRNA programmed to target SOD1 reduced mutant SOD1 mRNA and protein by 50-/5% in the spinal cord of a mouse model of SOD1-linked ALS, an outcome that we found decreased muscle atrophy, improved neuromuscular function and slowed the overall progression of the disease. As demonstration of its versatility for mediating knockdown in vivo, we further show, in certain embodiments, that RfxCas13d can be programmed to target HTT and that its intrastriatal delivery to a model of HD led to a potent reduction in mutant HTT protein and its toxic aggregates. In various embodiments, RfxCas13d can be utilized as a versatile platform for mediating RNA knockdown in the CNS.

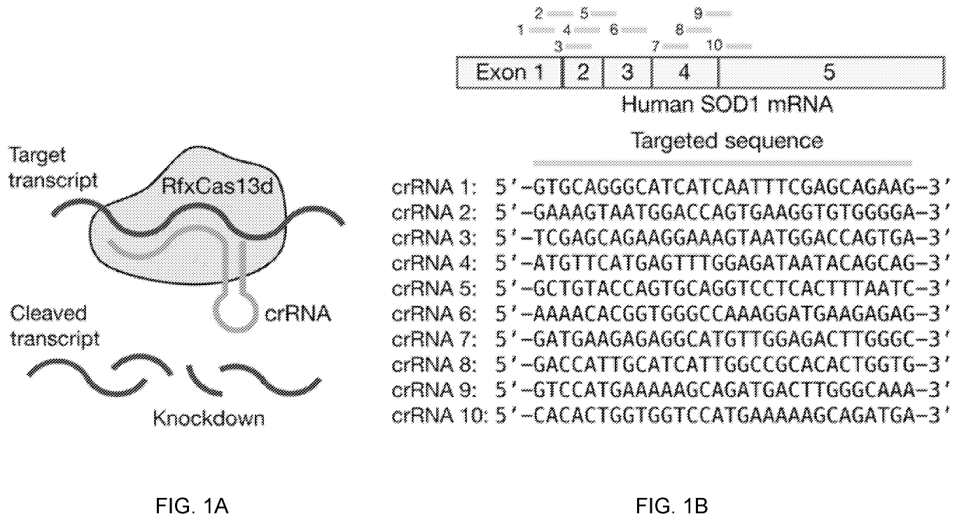

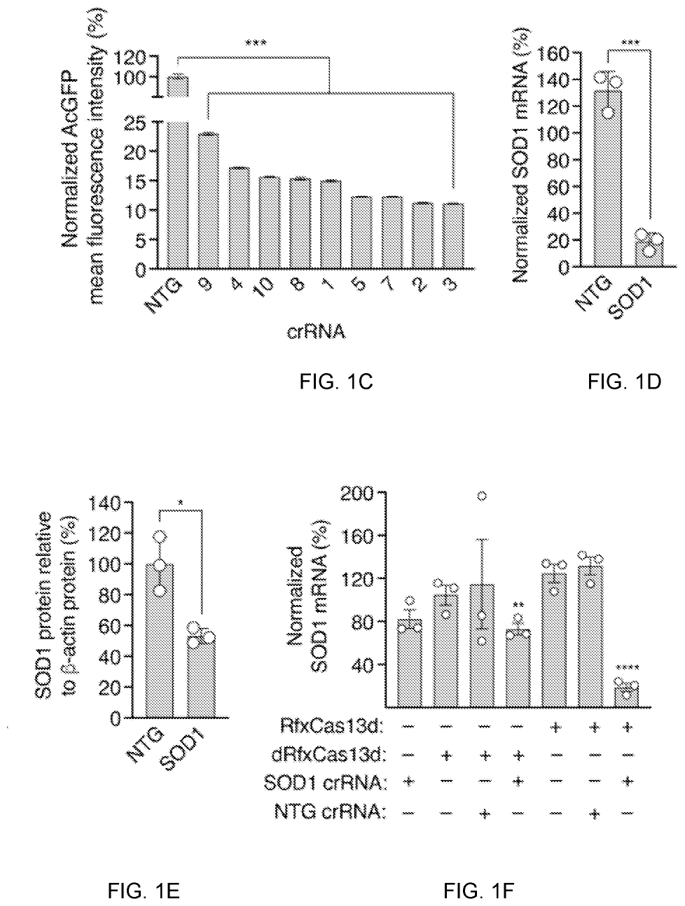

In an example, we sought to determine the ability for CRISPR-Cas13 to silence genes associated with neurodegenerative disorders. More specifically, we asked if RfxCas13d, a compact Cas13 variant with favorable targeting characteristics to other Cas13 orthologs, could target SOD1 (FIG. 1A), a ubiquitously expressed metalloenzyme that normally converts superoxide anions into oxygen and hydrogen peroxide and whose mutation can cause ALS (17), a rapidly progressive and paralytic disorder characterized by the selective loss of motor neurons in the spinal cord and brain (18). Given its causative role in ALS, lowering the expression of mutant SOD1 by gene silencing has emerged as a promising strategy for treating the disease (19-21).

To this end, we designed ten crRNAs with 30 nucleotide (nt) protospacers to target the mature human SOD1 mRNA (FIG. 1B). crRNA1 is SEQ ID NO:58; crRNA2 is SEQ ID NO:59; crRNA3 is SEQ ID NO:60; crRNA4 is SEQ ID NO:61; crRNA5 is SEQ ID NO:62; crRNA6 is SEQ ID NO: 63; crRNA7 is SEQ ID NO:64; crRNA8 is SEQ ID NO:65; crRNA9 is SEQ ID NO:66; crRNA10 is SEQ ID NO:67. Importantly, these crRNAs do not overlap with any common mutations in SOD1, ensuring they could be used in a mutation-independent manner to target the majority of the >100 different SOD1 mutations identified in ALS patients to date. Additionally, as Cas13d variants with a nuclear localization signal (NLS) sequence target RNA with greater efficiency than wild-type proteins, we used a RfxCas13d variant with NLS sequences at its N and C termini for our studies.

We first determined the ability of RfxCas13d to target SOD1 in human embryonic kidney (HEK) 293T cells. To identify the most active crRNAs, we constructed and used a reporter plasmid expressing complementary DNA (cDNA) encoding wild-type SOD1 fused to the green fluorescent protein from Aequorea coerulescens (AcGFP), thereby linking SOD1 expression to GFP fluorescence. According to flow cytometry, we found that all ten of the SOD1-targeting crRNAs decreased AcGFP fluorescence by >75% relative to a non-targeted crRNA (P<0.001), with the most active crRNAs observed to decrease fluorescence by ˜90% (P<0.001) (FIG. 1C), illustrating the overall programmability of RfxCas13d.

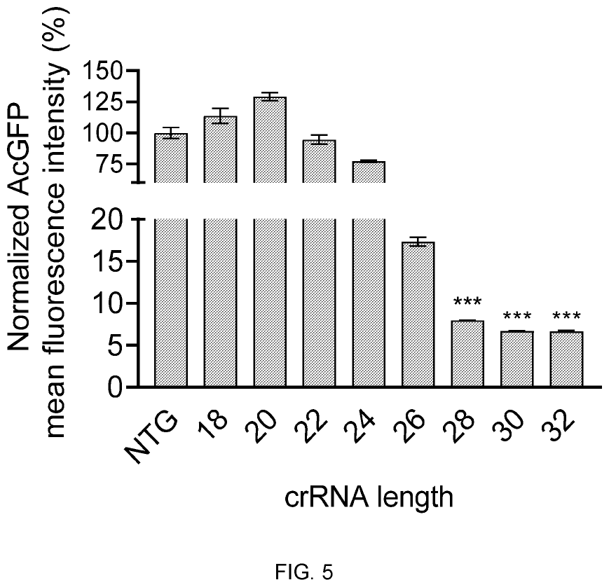

As the protospacer length of a crkNA can influence RtxCas13d targeting, we analyzed the activities of crRNAs with variously sized targeting sequences (18, 20, 22, 24, 26, 28, 30 and 32 nts) for their ability to silence the SOD1 reporter. While spacer sequences equal to or less than 24 nts displayed modest or no activity, crRNAs with protospacers exceeding 28 nts all had virtually identical targeting activities (˜95% decrease in GFP fluorescence) (FIG. 5). Given the lack of clear enhancement in targeting with alternatively sized crRNAs, we used a 30 nt protospacer for all subsequent studies.

We next evaluated the ability of RfxCas13d to target endogenous SOD1 mRNA in HEK293T cells. According to RT-qPCR and relative to untreated cells, we observed a ˜80% decrease in SOD1 mRNA at 72 hr post-transfection using crRNA 3 (P<0.001) (FIG. 1D), the most active crRNA identified from our reporter screen. Western blot corroborated this, revealing a two-fold reduction in total SOD1 protein at the same time point (FIG. 1E), demonstrating that RfxCas13d can knockdown SOD1 mRNA and protein.

As crRNAs have been reported as capable of knocking down genes even in the absence of a Cas13 protein (22), we also tested whether the SOD1-targeting crRNA could lower SOD1 either by itself (that is, without RfxCas13d) or with a catalytically inactivated RfxCas13d variant (dRfxCas13d). According to qPCR and relative to untreated cells, we measured a ˜18% decrease in SOD1 mRNA in cells transfected with just the SOD1-targeting crRNA (P=0.1) and a ˜28% decrease in SOD1 mRNA in cells transfected with dRfxCas13d and the SOD1-targeting crRNA (P=0.001; FIG. 1F), supporting the idea that Cas13 binding to an RNA target is sufficient to lead to a measurable knockdown. As additional controls, we assessed the ability for RfxCas13d alone, dRfxCas13d alone, RfxCas13d with a non-targeted crRNA and dRfxCas13d with a non-targeted crRNA to affect SOD1. No significant changes in SOD1 mRNA were observed for any of these combinations (FIG. 1F). Thus, our results demonstrate that RfxCas13d can be programmed to target SOD1 and that, while largely driven by the catalytic activity of RfxCas13d, non-RNase mechanism(s) could contribute to knockdown, potentially in a synergistic manner.

### Example 2: RfxCas13d Silencing of SOD1 Improves Therapeutic Outcomes in a Rodent Model of ALS

We next sought to determine if RfxCas13d could provide therapeutic benefit to a mouse model of SOD1-linked ALS following its in vivo delivery to the spinal cord by AAV.

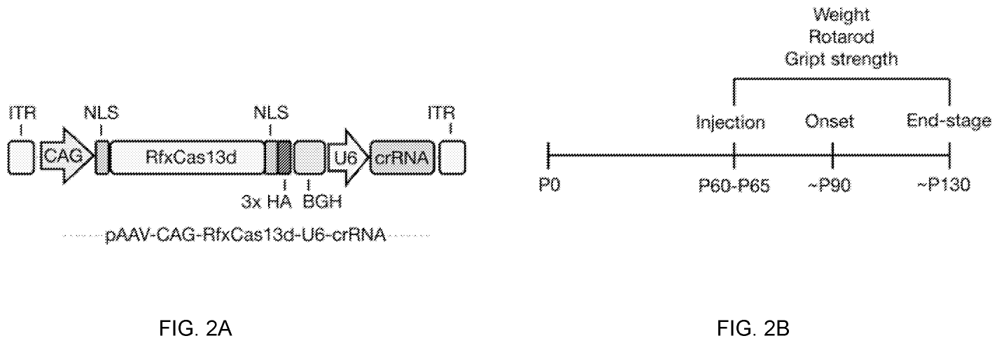

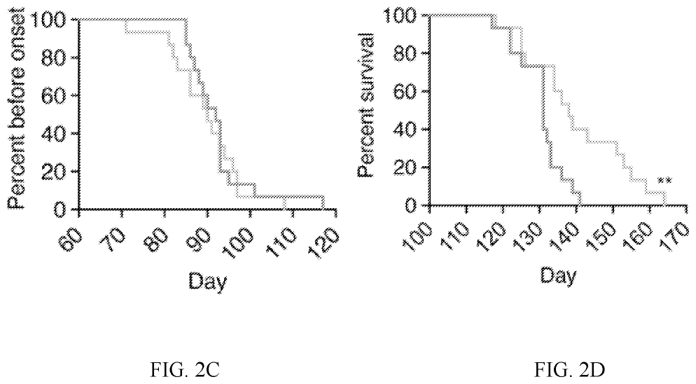

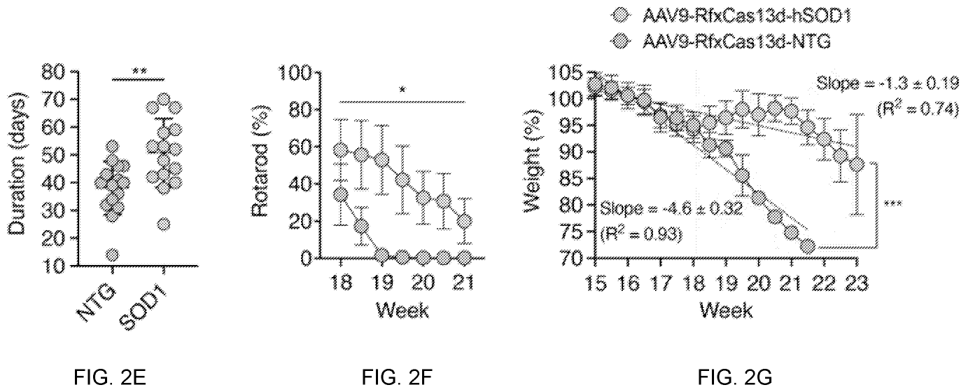

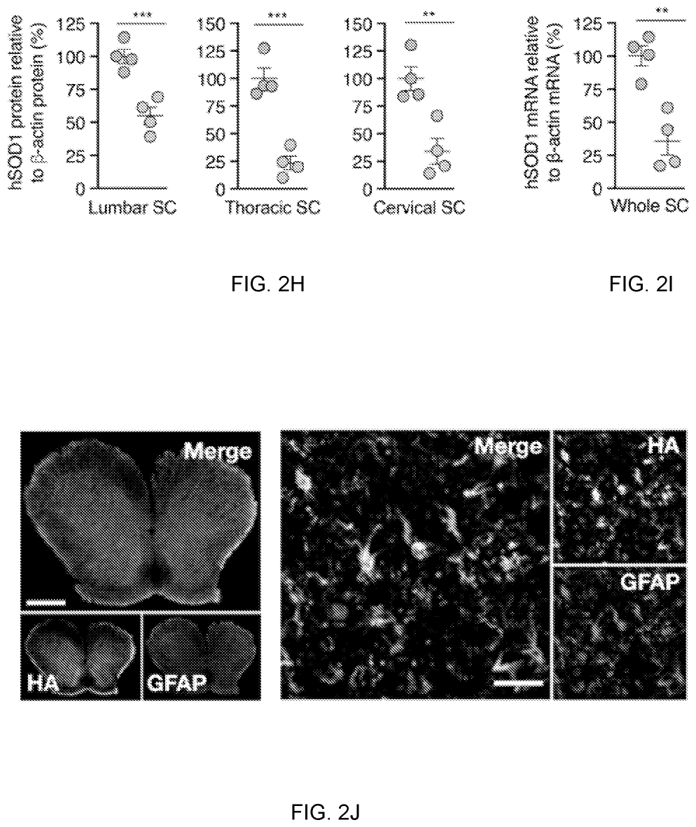

Importantly, while motor neurons are selectively lost in ALS, lowering the expression of mutant SOD1 within these cells only delays the onset of the disease, whereas decreasing its expression in astrocytes, a type of glial cell involved in the neuroinflammatory response, markedly slows the overall progression of the disorder. For these reasons, and because slowing disease progression is a clinically relevant outcome, we sought to deliver RfxCas13d to spinal cord astrocytes, a cell type that can be efficiently targeted in adult mice by AAV9. To this end, we injected 60- to 65-day-old G93A-SOD1 mice—which carry ˜25 copies of a human SOD1^{G93A} transgene and develop an aggressive disease that phenocopies hallmarks of ALS, including motor neuron degeneration, muscle wasting, and paralysis—with AAV9 vector encoding RfxCas13d with either the SOD1-targeting crRNA (AAV9-RfxCas13d-hSOD1) or a non-targeted crRNA (AAV9-RfxCas13d-NTG) via the cerebrospinal fluid (CSF) of the lumbar spine (FIG. 2A, B).

As expected, given the strong preference that intrathecally administered AAV9 has for spinal cord astrocytes and the fact that disease onset in SOD1-ALS mice is modulated by the expression of mutant SOD1 in motor neurons, we observed no delay in the onset of the disease (measured as peak weight) in mice injected with AAV9-RfxCas13d-hSOD1 versus the controls (FIG. 2C). However, as compared to control animals, we observed that mice treated by RfxCas13d had both significantly increased survival (P<0.01; FIG. 2D) and a considerably slower course of disease, evidenced by a ˜33% increase in the duration of disease, a measure defined as the length of time between disease onset and end-stage (hSOD1: 50.7±12.4 days; NTG: 38.1±9.6 days; P<0.001; FIG. 2E).

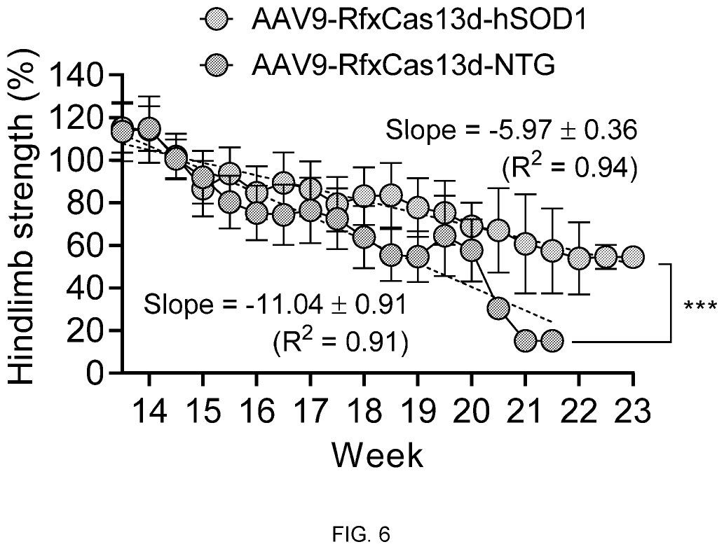

In addition to the overall slowing of disease, we observed that G93A-SOD1 mice treated by RfxCas13d had improved functional outcomes, including significantly improved rotarod times during the late stage of the disease (P<0.05) (FIG. 2F), a period defined as the time from when animals lose 10% of their peak weight to end-stage, as well as a two-fold slowing in the rate with which hind limb grip strength declined over the course of the entire disease (FIG. 6), which we determined by a linear regression analysis from a long-term longitudinal assessment of hindlimb strength (hSOD1: −6.0±0.36 normalized strength % per measurement; NTG: −11.0±0.91 normalized strength % per measurement; P<0.001). Linear regression analysis of biweekly weight measurements further revealed that mice injected with AAV9-RfxCas13d-hSOD1 had a nearly three-fold slower rate of muscle atrophy than control mice (hSOD1: −1.3±0.19 weight % per measurement; NTG: −4.6±0.32 weight % per measurement; P<0.001; FIG. 2G), which altogether demonstrates that RfxCas13d can improve therapeutic outcomes in SOD1-ALS mice.

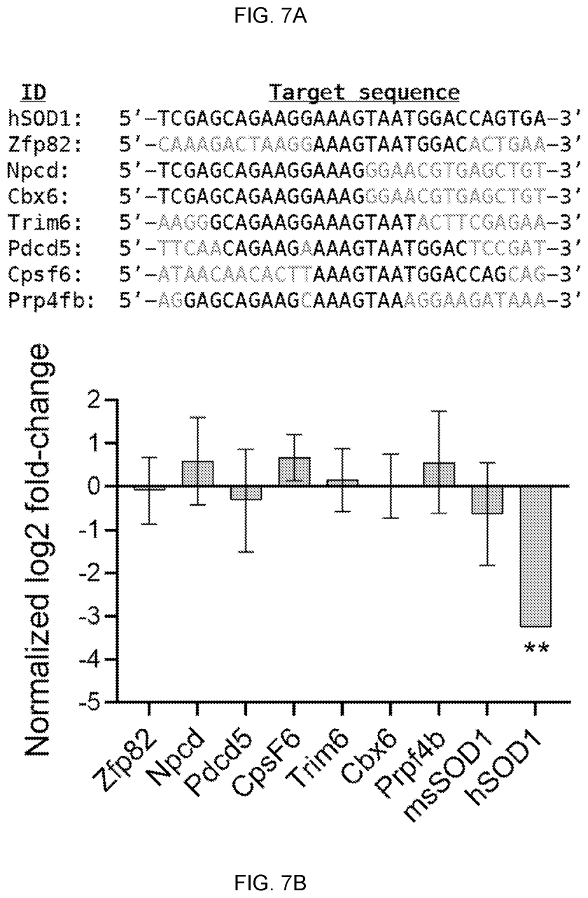

We next quantified SOD1 silencing in tissue harvested from end-stage animals. According to western blot, G93A-SOD1 mice injected with AAV9-RfxCas13d-hSOD1 had reduced mutant SOD1 protein in each region of the spinal cord compared to controls (FIG. 2H), including a 65-75% reduction in mutant protein in the cervical and thoracic regions (P<0.001), and a ˜45% reduction of mutant protein in the lumbar region (P<0.01). Consistent with this finding, we also observed a ˜65% decrease in the relative amount of SOD1 mRNA in the whole spinal cord of animals treated by RfxCas13d compared to the controls (P<0.001) (FIG. 2I), demonstrating that RfxCas13d can lower SOD1 in vivo. To evaluate off-target silencing by RfxCas13d, we next analyzed the expression of six candidate off-target transcripts, which each contained a potential pseudo-crRNA binding site with at least 15 nucleotides of homology to the SOD1-targeting crRNA (FIG. 7A). Compared to control mice, no significant differences in expression (all P>0.1) were measured by qPCR for any of the candidate genes in whole spinal cord tissue from mice injected with AAV9-RfxCas13d-hSOD1 (FIG. 7B).

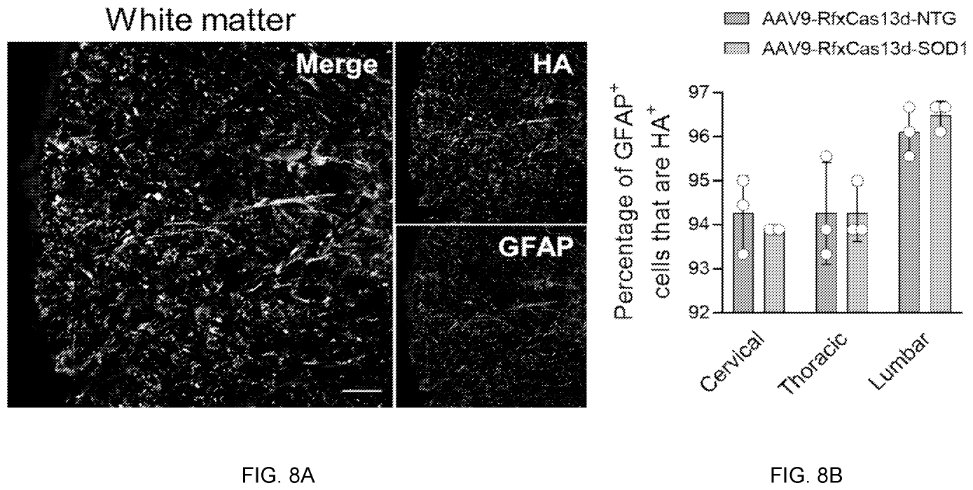

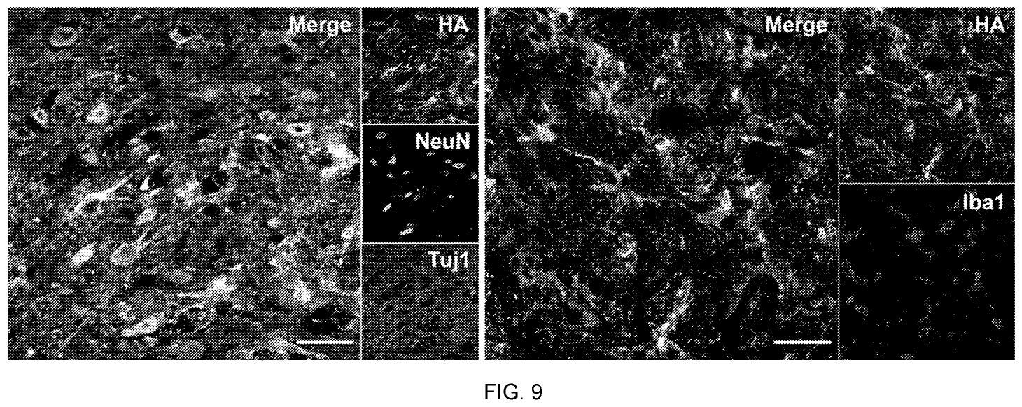

We next analyzed the distribution of RfxCas13d protein in G93A-SOD1 mice. According to an immunofluorescent analysis, we detected RfxCas13d in ˜90% of analyzed GFAP⁺ astrocytes in the anterior horn of the lumbar, thoracic and cervical spinal cord of end-stage animals (FIG. 2J; FIG. 8). Consistent with the preference that intrathecally delivered AAV9 has for spinal cord astrocytes, we observed limited RfxCas13d expression in other cell types within the spinal cord, including Iba1⁺ microglia and NeuN⁺ neurons (FIG. 9).

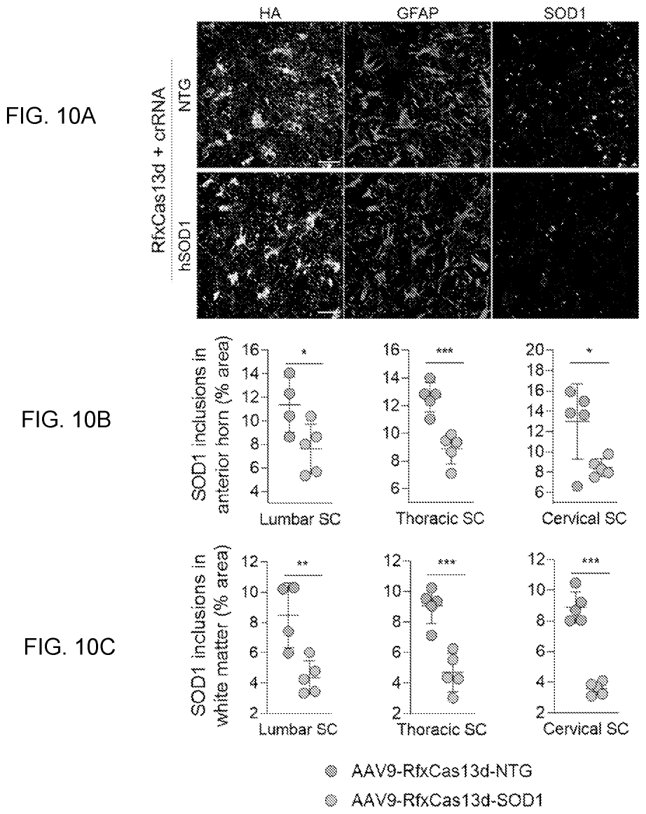

Finally, we quantified if RfxCas13d reduced the accumulation of SOD1 immunoreactive inclusions in the spinal cord, an outcome that we and others have observed can occur in SOD1-ALS mice after mutant SOD1 expression is ablated in spinal cord astrocytes. Based on fluorescent immunostaining, we found that mice injected with AAV9-RfxCas13d-hSOD1 had on average ˜40% fewer SOD1 immunoreactive inclusions in the spinal cord white matter and ˜29% fewer immunoreactive inclusions in the spinal cord anterior horn (P<0.05 for all; FIG. 10).

In sum, our results demonstrate that RfxCas13d can impact a therapeutic benefit to a mouse model of SOD1-linked ALS by knocking down mutant SOD1 mRNA and protein in the spinal cord.

### Example 3: Lowering Huntingtin by RfxCas13d Targeting

We next sought to determine the general applicability of RfxCas13d targeting in the CNS. To this end, we asked if RfxCas13d could be used to knockdown huntingtin (HTT), a protein with a polyglutamine (polyQ) tract near its N-terminus that, when expanded beyond 37 repeats, causes Huntington's disease (HD), a progressive and ultimately fatal neurodegenerative disorder characterized by the loss of neurons in the striatum. Given the causal link between mutant HTT and HD, modalities capable of lowering mutant HTT gene expression hold the potential to slow the clinical progression of the disease.

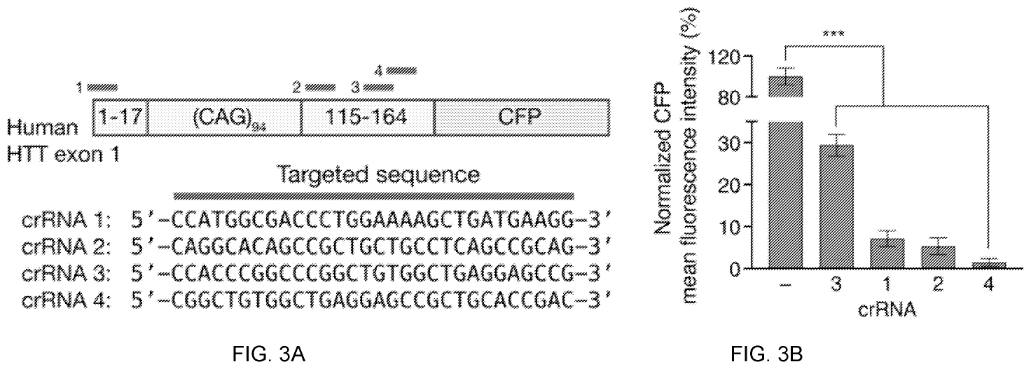

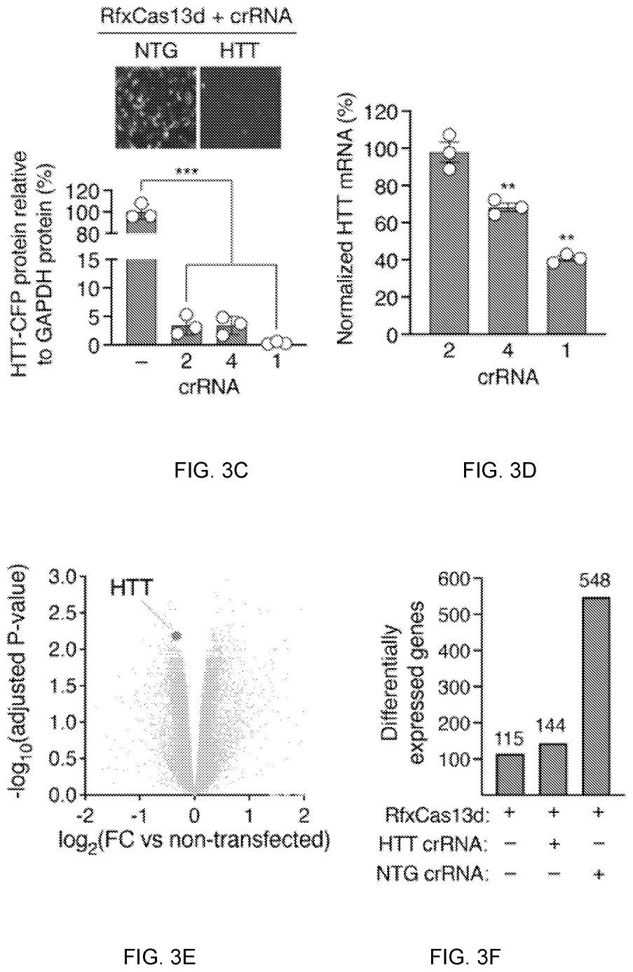

Based on the versatility and flexibility observed by RfxCas13d for SOD1, we designed just four crRNAs to target the sequence corresponding to exon 1 of the human HTT mRNA, both upstream and downstream of the polyQ-encoding repeat expansion (FIG. 3A). We then evaluated the silencing activity of the crRNAs in HEK293T cells using an inducible reporter that expresses exon 1 of the human HTT gene with 94Q fused to cyan fluorescent protein (CFP), thus linking the expression of HTT exon 1 to CFP fluorescence (FIG. 3A).

According to flow cytometry, all four of the crRNAs reduced CHP expression by >70% relative to a non-targeting crRNA, with the three most active crRNAs found to decrease fluorescence by ˜95% (P<0.001; FIG. 3B). As this reporter expresses a mutant HTT protein fragment that also forms fluorescent inclusions in cells, we used fluorescence microscopy to analyze aggregate formation, observing a dramatic reduction in the number of visible inclusions in cells with RfxCas13d and the most active crRNA (FIG. 3C). Western blot confirmed this reduction, revealing at least an 80% knockdown in mutant protein for each of the three most active crRNAs (FIG. 3C).

We next sought to determine the ability of RfxCas13d to target the endogenous HTT mRNA in HEK293T cells. By qPCR, we measured a significant decrease in HTT mRNA for two crRNAs, with the more active crRNA, crRNA 1, found to reduce HTT mRNA by ˜60% in comparison to cells transfected with RfxCas13d and a non-targeting crRNA (P<0.001; FIG. 3D), demonstrating that RfxCas13d can be programmed to target endogenous HTT mRNA in human cells.

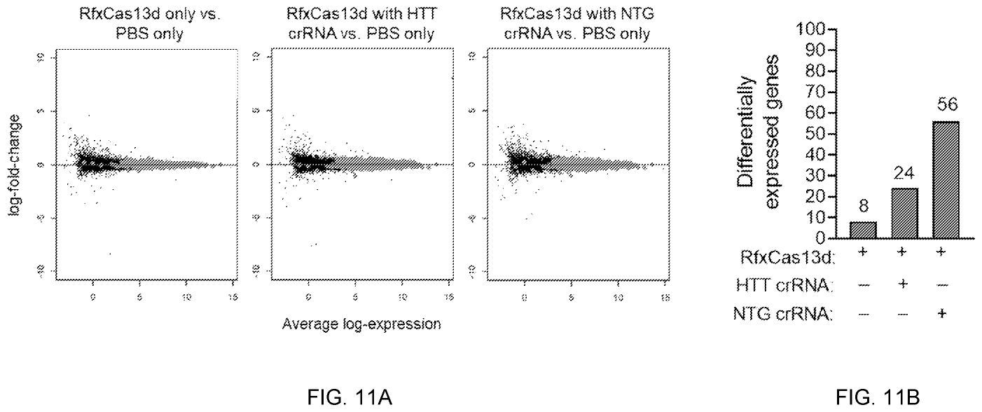

As RfxCas13d has been reported as capable of affecting the expression of non-target transcripts, we next analyzed on a transcriptome-wide scale the targeting specificity of RfxCas13d with the HTT-targeting crRNA in HEK293T cells. Using RNA-seq, we found that, in addition to HTT, whose downregulation was expected, 144 genes (˜0.92% of total measured transcripts) were differentially expressed in cells transfected with RfxCas13d and the HTT crRNA (>1.25-fold change; FDR P value<0.01) (FIG. 3E). For reference, RfxCas13d alone or with a non-targeted crRNA affected the expression of ˜0.73% and ˜3.5% of total transcripts, respectively (FIG. 3F). To further probe if RfxCas13d affected cell homeostasis, we examined the effect that RfxCas13d had on the expression of 2,134 constitutively expressed housekeeping (HK) genes. Overall, we observed a similar frequency of differentially expressed genes as compared to the whole-transcriptomes analysis: ˜ 0.37%, ˜1.1%, and ˜2.6% of HK genes were affected in cells transfected with RfxCas13d alone, RfxCas13d with HTT crRNA, and RfxCas13d with non-targeted crRNA, respectively (>1.25-fold change; FDR P value<0.01) (FIG. 11), demonstrating that RfxCas13d targeted HTT mRNA without significantly affecting the expression of very many genes.

Finally, we sought to determine whether RfxCas13d could lower the mutant human HTT protein in a transgenic mouse model of HD, specifically R6/2 mice, which carry exon 1 of the human HTT gene with a ˜150 polyQ repeat alongside a ˜1 kilobase fragment of the 5′ untranslated region (UTR) that drives expression of the transgene. R6/2 mice produce the toxic N-terminal fragment of the human mutant HTT protein and develop inclusions in striatal neurons as early as 4 weeks of age and are thus a useful model for evaluating the activity of HTT silencing agents.

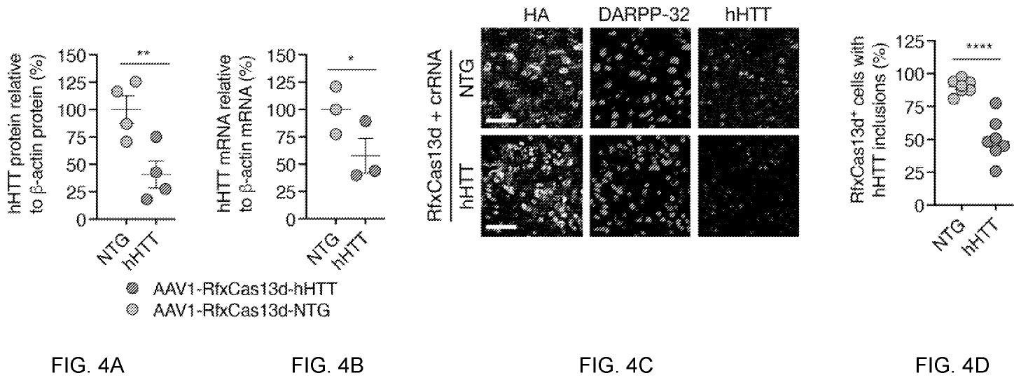

At four weeks after bilaterally injecting the striata of R6/2 mice with AAV1 vector encoding RfxCas13d with either the HTT-targeting crRNA (AAV1-RfxCas13d-hHTT, left hemisphere) or a non-targeted crRNA (AAV1-RfxCas13d-NTG, right hemisphere), we quantified the abundance of the human mutant HTT protein in dissected striatal tissue. According to western blot and compared to control tissue, we measured a ˜60% decrease in mutant HTT protein in hemispheres injected with AAV1-RfxCas13d-hHTT (P<0.05) (FIG. 4A), a finding that was corroborated by qPCR, which revealed a ˜45% reduction in the amount of relative human HTT mRNA in treated hemispheres compared to control hemispheres (P<0.05) (FIG. 4B). As R6/2 mice develop HTT immunoreactive inclusions, we next used immunohistochemistry to determine if RfxCas13d could reduce their abundance in the brain. In addition to observing RfxCas13d expression in cells positive for the medium-spiny neuron marker DARPP-32⁺ (FIG. 4C), the principal cell type lost in HD, we measured that hemispheres injected with AAV1-RfxCas13d-hHTT had ˜50% fewer RfxCas13d⁺ cells with HTT immunoreactive inclusions as compared to RfxCas13d⁺ cells from the control hemisphere (P<0.0001) (FIG. 4C, D), demonstrating that RfxCas13d can reduce a pathology associated with HD.

In sum, our results establish that RfxCas13d can be delivered to the spinal cord and brain and knockdown neurodegeneration-associated genes.

### Example 4: Discussion

By virtue of their high programmability and their ability to cleave targeted RNAs via an intrinsic RNase activity, CRISPR-Cas13 effectors can mediate RNA knockdown in mammalian cells and hold potential for therapeutic gene silencing. In the present study we demonstrate that RfxCas13d, a Cas13 effector that is compact enough to fit within a single AAV vector particle alongside a crRNA expression cassette, can be programmed to target SOD1, a protein whose mutation has been linked to inherited forms of ALS, and HTT, a protein that, when mutated to carry an expansion of a polyQ stretch in its N-terminus, causes HD. Our results thus establish RfxCas13d as a versatile platform for suppressing mutant gene function in the nervous system.

Gene silencing has emerged as a promising strategy for treating various CNS disorders. As evidence for this, tofersen, an antisense oligonucleotide (ASO) for SOD1, has advanced to phase 3 trials (ClinicalTrials.gov Identifier: NCT02623699), while miRNA-based approaches for SOD1-ALS and HD (ClinicalTrials.gov Identifier: NCT04120493) are under evaluation in patients. Yet despite their broad potential, traditional gene silencing modalities possess certain limitations. For instance, ASOs have a transient lifecycle and will require a lifetime of administrations, which, in addition to potentially producing periods of diminished activity before a re-dosing, could pose a physical and financial burden on patients. Conversely, while siRNAs, shRNAs and miRNAs can be expressed from a viral vector to continuously engage with a target RNA, these modalities rely on endogenous pathways whose activation can trigger the silencing of off-target transcripts. Thus, there remains a need to explore alternate RNA targeting platforms.

To this end, in addition to assessing its ability for mediating RNA knockdown in the CNS, we sought to determine the potential for Cas13d to improve therapeutic outcomes in a mouse model of a neurodegenerative disorder. Our results establish that Cas13d can in fact impact a therapeutic benefit, as we found that G93A-SOD1 mice intrathecally injected with AAV encoding Cas13d and a SOD1-targeting crRNA had a significantly slowed disease, improved survival, a decreased rate of muscle atrophy and improved rotarod and grip strength.

Surprisingly, our results also suggest that RNase-independent mechanisms can contribute to RNA knockdown, as we observed a measurable decrease in SOD1 mRNA in cells transfected with just the SOD1-targeting crRNA or a catalytically dead variant of RfxCas13d with the SOD1-targeting crRNA, bringing to light the possibility that there may exist enzymatic and non-enzymatic mechanisms working in concert to knock down RNA. Our results demonstrate that, on a transcriptome-wide scale in a human cellular background, Cas13 can knockdown a target RNA without significantly affecting the expression of very many genes.

We implemented non-allele-specific strategies to target both SOD1 and HTT, which holds the advantage of being applicable to a wider patient population than mutation-specific approaches for these disorders. For instance, in the case of SOD1, >150 different mutations have been identified in ALS patients while, for HTT, even the most prevalent mutant allele-specific SNPs are estimated to be present in only ˜20% of the HD patient population. To this end, non-allele-specific strategies have been implemented with ASOs and shRNA/miRNAs for both SOD1-linked ALS and HD. However, these approaches are also expected to reduce the wild-type protein, which hold risks for inducing adverse effects. While, to date, non-allele-specific targeting for SOD1-linked ALS has resulted in no serious side effects and produced evidence of clinical improvement, a phase 3 clinical trial designed to evaluate the effectiveness of an intrathecally administered non-allele-specific ASO for HD was recently halted, as it failed to show greater efficacy than a placebo. However, a phase 1/2 trial for an AAV-based non-allele-specific miRNA therapy for HD involving its surgical delivery to the striatum is ongoing (ClinicalTrials.gov: NCT04120493).

In conclusion, we have demonstrated that CRISPR-Cas13 can be delivered to the nervous system to knockdown neurodegeneration-associated genes. Our findings support its use as an agent for therapeutic gene silencing.

### Example 5: Materials and Methods

### Plasmid Construction

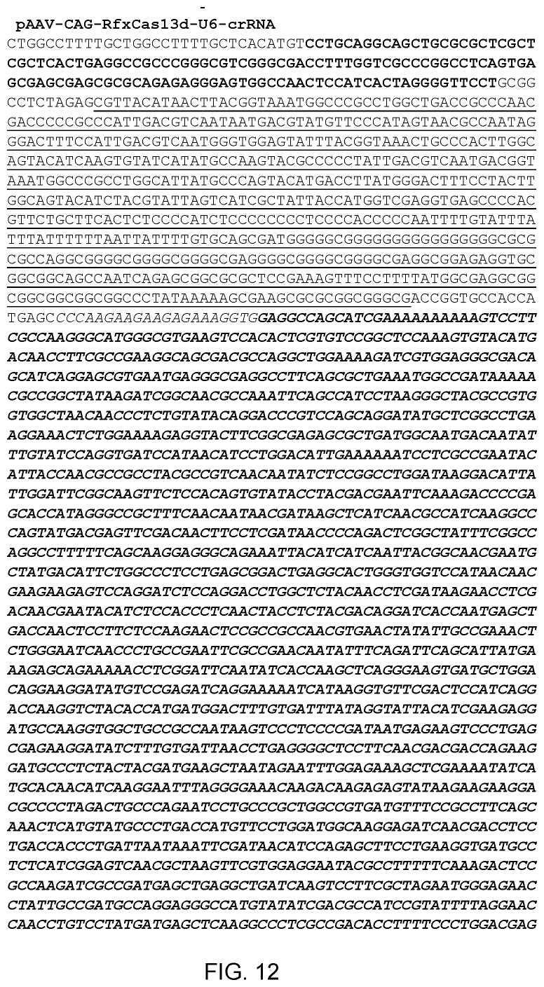

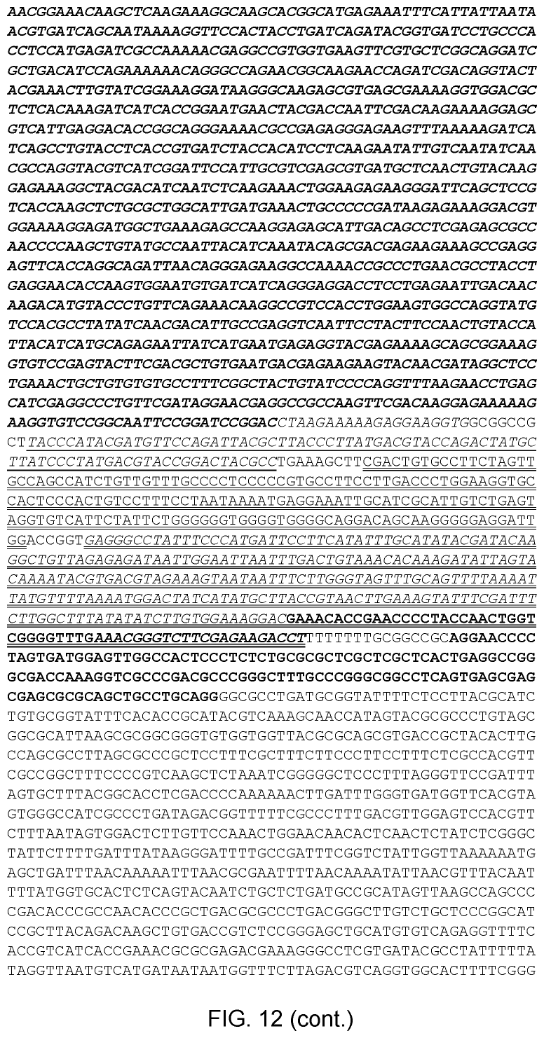

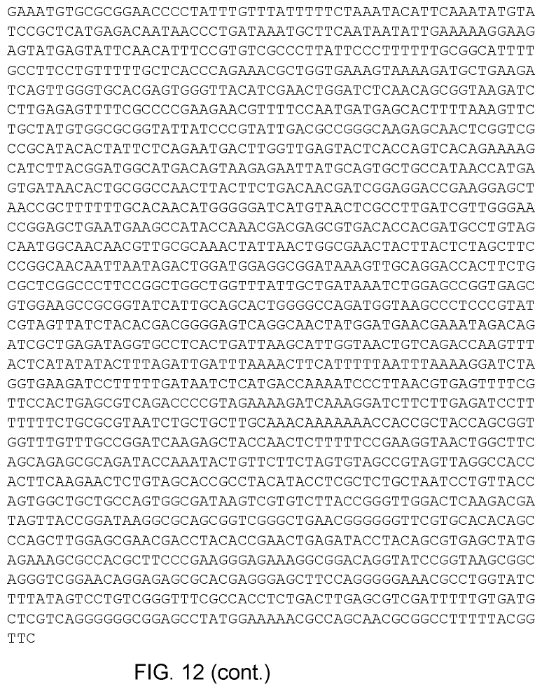

pXR001, the plasmid encoding RtxCas13d (Addgene, 109049), and pXR003, the plasmid encoding the crRNA expression cassette (Addgene, 109053) were gifts from Patrick Hsu. To clone the pAAV plasmid pAAV-CAG-RfxCas13d-U6-crRNA, (1) RfxCas13d, (2) three tandem copies of an HA epitope tag, and BGH-poly(A) polyadenylation signal, and (3) the human U6 promoter with a crRNA scaffold were PCR amplified from (1) pXR001, (2) pAAV-CAG-C-Int-CBE-U6-sgRNA and (3) pXR003, respectively. These cassettes were then inserted between the AgeI and NotI restriction sites of the plasmid pAAV-CAG-C-Int-CBE-U6-sgRNA by a single-step Gibson assembly using the Gibson Assembly Master mix (New England Biolabs, NEB) according to the manufacturer's instructions. Sanger sequencing (ACGT) was used to confirm the sequence of the plasmid. The full sequence of pAAV-CAG-RfxCas13d-U6-crRNA is shown in FIG. 12.

To construct the SOD1-AcGFP reporter plasmid, site-directed mutagenesis was performed on pF148-pSOD1-G37R-AcGFP1 (Addgene, 26409), a gift from Elizabeth Fisher. Briefly, primers encoding nucleotides to revert the SOD1-G37R mutation back to wild-type were used to amplify pF148-pSOD1-G37R-AcGFP1 using Phusion High-Fidelity DNA Polymerase (NEB). The PCR reaction was then incubated with 1 μL of DpnI (NEB) for 1 hr at 37° C. and transformed to 5-alpha Competent Escherichia coli (NEB). Sanger sequencing (ACGT) was used to confirm the sequence of the plasmid.

Oligonucleotides encoding the crRNA targeting sequences were custom synthesized (Integrated DNA Technologies, IDT) and then incubated with T4 polynucleotide kinase (New England Biolabs; NEB) for 30 min at 37° C., annealed at 95° C. for 5 min and then cooled to 4° C. at a rate of −0.1° C./s. Annealed oligonucleotides were then ligated into the BbsI restriction sites in pXR003 or pAAV-CAG-RfxCas13d-U6-crRNA. Sanger sequencing (ACGT) was used to confirm the sequence of the crRNA.

All primer sequences are shown in Table 1.

| Name | Sequence (5′ to 3′) |
|---|---|
| hSOD1 crRNA 1 Fwd | AAACCTTCTGCTCGAAATTGATGATGCCCTGCAC (SEQ ID NO: 4) |
| hSOD1 crRNA 1 Rev | AAAAGTGCAGGGCATCATCAATTTCGAGCAGAAG (SEQ ID NO: 5) |
| hSOD1 crRNA 2 Fwd | AAACTCCCCACACCTTCACTGGTCCATTACTTTC (SEQ ID NO: 6) |
| hSOD1 crRNA 2 Rev | AAAAGAAAGTAATGGACCAGTGAAGGTGTGGGGA (SEQ ID NO: 7) |
| hSOD1 crRNA 3 Fwd | AAACTCACTGGTCCATTACTTTCCTTCTGCTCGA (SEQ ID NO: 8) |
| hSOD1 crRNA 3 Rev | AAAATCGAGCAGAAGGAAAGTAATGGACCAGTGA (SEQ ID NO: 9) |
| hSOD1 crRNA 4 Fwd | AAACATTATCTCCAAACTCATGAACATGGAATCC (SEQ ID NO: 10) |
| hSOD1 crRNA 4 Rev | AAAAGGATTCCATGTTCATGAGTTTGGAGATAAT (SEQ ID NO: 11) |
| hSOD1 crRNA 5 Fwd | AAACCATCCTTTGGCCCACCGTGTTTTCTGGATA (SEQ ID NO: 12) |
| hSOD1 crRNA 5 Rev | AAAATATCCAGAAAACACGGTGGGCCAAAGGATG (SEQ ID NO: 13) |
| hSOD1 crRNA 6 Fwd | AAACCTCTCTTCATCCTTTGGCCCACCGTGTTTT (SEQ ID NO: 14) |
| hSOD1 crRNA 6 Rev | AAAAAAAACACGGTGGGCCAAAGGATGAAGAGAG (SEQ ID NO: 15) |
| hSOD1 crRNA 7 Fwd | AAACGCCCAAGTCTCCAACATGCCTCTCTTCATC (SEQ ID NO: 16) |
| hSOD1 crRNA 7 Rev | AAAAGATGAAGAGAGGCATGTTGGAGACTTGGGC (SEQ ID NO: 17) |
| hSOD1 crRNA 8 Fwd | AAACCACCAGTGTGCGGCCAATGATGCAATGGTC (SEQ ID NO: 18) |
| hSOD1 crRNA 8 Rev | AAAAGACCATTGCATCATTGGCCGCACACTGGTG (SEQ ID NO: 19) |
| hSOD1 crRNA 9 Fwd | AAACTTTGCCCAAGTCATCTGCTTTTTCATGGAC (SEQ ID NO: 20) |
| hSOD1 crRNA 9 Rev | AAAAGTCCATGAAAAAGCAGATGACTTGGGCAAA (SEQ ID NO: 21) |
| hSOD1 crRNA 10 Fwd | AAACTCATCTGCTTTTTCATGGACCACCAGTGTG (SEQ ID NO: 22) |
| hSOD1 crRNA 10 Rev | AAAACACACTGGTGGTCCATGAAAAAGCAGATGA (SEQ ID NO: 23) |
| HTT crRNA 1 Fwd | AAACCCTTCATCAGCTTTTCCAGGGTCGCCATGG (SEQ ID NO: 24) |
| HTT crRNA 1 Rev | AAAACCATGGCGACCCTGGAAAAGCTGATGAAGG (SEQ ID NO: 25) |
| HTT crRNA 2 Fwd | AAACCTGCGGCTGAGGCAGCAGCGGCTGTGCCTG (SEQ ID NO: 26) |
| HTT crRNA 2 Rev | AAAACAGGCACAGCCGCTGCTGCCTCAGCCGCAG (SEQ ID NO: 27) |
| HTT crRNA 3 Fwd | AAACCGGCTCCTCAGCCACAGCCGGGCCGGGTGG (SEQ ID NO: 28) |
| HTT crRNA 3 Rev | AAAACCACCCGGCCCGGCTGTGGCTGAGGAGCCG (SEQ ID NO: 29) |
| HTT crRNA 4 Fwd | AAACGTCGGTGCAGCGGCTCCTCAGCCACAGCCG (SEQ ID NO: 30) |
| HTT crRNA 4 Rev | AAAACGGCTGTGGCTGAGGAGCCGCTGCACCGAC (SEQ ID NO: 31) |
| qPCR-hSOD1-Fwd | CAATGTGACTGCTGACAAAG (SEQ ID NO: 32) |
| qPCR-hSOD1-Rev | GTGCGGCCAATGATGCAAT (SEQ ID NO: 33) |
| qPCR-GAPDH-Fwd | CCTGACCTGCCGTCTAGAAAA (SEQ ID NO: 34) |
| qPCR-GAPDH-Rev | CTCCGACGCCTGCTTCAC (SEQ ID NO: 35) |
| qPCR-msBeta-Actin- | CACGATGGAGGGGCCGGACTCATC (SEQ ID NO: 36) |
| Fwd | |
| qPCR-msBeta-Actin- | TAAAGACCTCTATGCCAACACAGT (SEQ ID NO: 37) |
| Rev | |
| qPCR-HTT-Fwd | GGGTCCAAGATGGACGGC (SEQ ID NO: 38) |
| qPCR-HTT-Rev | AGCACCGGGGCAATGAATG (SEQ ID NO: 39) |
| qPCR-bGHpA-Fwd | GCCTTCTAGTTGCCAGCCAT (SEQ ID NO: 40) |
| qPCR-bGHpA-Rev | GGCACCTTCCAGGGTCAAG (SEQ ID NO: 41) |
| qPCR-Zfp82-Fwd | ACGGGGACAACTCACTCTC (SEQ ID NO: 42) |
| qPCR-Zfp82-Rev | ACGGGGACAACTCACTCTC (SEQ ID NO: 43) |
| qPCR-Npcd-Fwd | TGGGCGATCAAGTACAGCAC (SEQ ID NO: 44) |
| qPCR-Npcd-Rev | TTTGGGTTTAGGTCCCCTCTT (SEQ ID NO: 45) |
| qPCR-Cbx6-Fwd | TGGCACATTTGCGCTCTACAA (SEQ ID NO: 46) |
| qPCR-Cbx6-Rev | CTCCGGGAGTAGCTTGGGT (SEQ ID NO: 47) |
| qPCR-Trim6-Fwd | ATGACTTCAACAGTCTTGGTGG (SEQ ID NO: 48) |
| qPCR-Trim6-Rev | TTCCCAGGCTGATAGGAGGTC (SEQ ID NO: 49) |
| qPCR-Pdcd5-Fwd | ATGGCGGACGAAGAACTTGAG (SEQ ID NO: 50) |
| qPCR-Pdcd5-Rev | CGGGCTGACTGATCCAGAA (SEQ ID NO: 51) |
| qPCR-Cpsf6-Fwd | CATCTGCAAATAATGGCGATGC (SEQ ID NO: 52) |
| qPCR-Cpsf6-Rev | CTCATCTGTTGTCCACCATGTTA (SEQ ID NO: 53) |
| qPCR-Prpf4b-Fwd | ATCGAGGCTTCCGACAAAGAA (SEQ ID NO: 54) |
| qPCR-Prpf4b-Rev | TGCCTTTTCGTGAATCTCTCCT (SEQ ID NO: 55) |
| qPCR-msSOD1-Fwd | ATGGCGATGAAAGCGGTGT (SEQ ID NO: 56) |
| qPCR-msSOD1-Rev | CCTTGTGTATTGTCCCCATACTG (SEQ ID NO: 57) |

### Cell Culture and Transfections

HEK293T cells (ATCC) were maintained in Dulbecco's Modified Eagle's Medium (DMEM; Corning) supplemented with 10% (v/v) fetal bovine serum (FBS; ThermoFisher Scientific) and 1% (v/v) antibiotic-antimycotic (ThermoFisher Scientific) in a humidified 5% CO₂ incubator at 37° C. Cells were seeded onto 24-well plates at an average density of 2×10⁵ cells per well and transfected with 1 μg of pAAV-CAG-RfxCas13d-U6-crRNA by Lipofectamine 3000 (ThermoFisher Scientific), according to the manufacturer's instructions.

For flow cytometry using the SOD1-AcGFP reporter plasmid, cells were transfected with 800 ng of pAAV-CAG-RfxCas13d-U6-crRNA and 200 ng of SOD1-AcGFP reporter plasmid, while, for flow cytometry using the HTT-CFP reporter plasmid, cells were transfected with 800 ng of pAAV-CAG-RfxCas13d-U6-crRNA, 100 ng tTA/TRE-mCherry and 100 ng of pTreTight-HTT94Q-CFP (Addgene, 23966).

### Flow Cytometry

At 72 hr after transfection, cells were harvested, washed with PBS, and strained into single-cell suspensions using Falcon Round-Bottom Polystyrene Test Tubes with Cell Strainer Snap Caps. Fluorescence was then measuring using a BD LSR Fortessa Flow Cytometry Analyzer (Roy J. Carver Biotechnology Center Flow Cytometry Facility). 50,000 events were recorded for each sample, and data was analyzed using FlowJo v 10 (FlowJo, LLC).

qPCR

RNA was extracted from cells using the PureLink RNA Mini Kit (Invitrogen) and converted to cDNA using the iScript cDNA Synthesis Kit (BioRad) according to the manufacturers' instructions. 50 ng of cDNA was then used per qPCR reaction using iTaq Universal SYBR Green Supermix (Bio-Rad). qPCR measurements for each biological replicate were conducted in technical triplicates and normalized to human GAPDH or mouse β-actin expression for each respective sample. The average told change tor each sample was determined using the 2^{-ΔΔCT method.}

### Western Blot

Cells were lysed by RIPA buffer (0.2% IGEPAL CA-620, 0.02% SDS with VWR Life Science Protease Inhibitor Cocktail) and protein concentration was determined by using the DC Protein Assay Kit (Bio-Rad). 15 ug of protein was then electrophoresed by SDS-PAGE and electrophoretically transferred onto a polyvinylidene fluoride (PVDF) membrane in transfer buffer (20 mM Tris-HCl, 150 mM glycine and 20% [v/v] methanol) for 1.5 hr at 100V. Membranes were blocked with 5% (v/v) blotting-grade blocker (Bio-Rad) in Tris-buffered saline (TBS, 10 mM Tris-HCl, 150 mM NaCl, and 0.1%, pH) with 0.05% Tween 20 (TBS-T) for 1 hr and then incubated with primary antibodies in blocking solution at 4° C. overnight. The following primary antibodies were used: rabbit anti-hSOD1 (1:2,000; Cell Signaling Technology, 2770S), rabbit anti-GFP/CFP (1:1000, Abcam, ab6556), rabbit anti-GAPDH (1:1,000 Cell Signaling Technology, 2118S) and rabbit anti-β-actin (1:1000, Cell Signaling Technology, 4970S). Membranes were then washed three times with TBS-T and incubated with goat anti-rabbit horseradish peroxidase conjugate (1:4,000 ThermoFisher Scientific, 65-6120) in blocking solution for 1 hr at room temperature (RT). Membranes were then washed again three times with TBS-T and developed using SuperSignal West Dura Extended Duration Substrate (ThermoFisher Scientific) and visualized by automated chemiluminescence using the ChemiDoc XRS+ (Bio-Rad). Band intensity was quantified using Image Lab software (BioRad) and normalized to the reference protein in each line.

To analyze the N-terminal fragment of the human HTT protein, R6/2 striatal tissue was lysed using RIPA buffer and 20 ug of protein was electrophoresed on a 4-15% Mini-PROTEAN TGX Precast Protein Gel (Bio-Rad) and transferred onto a (PVDF) membrane in transfer buffer (20 mM Tris-HCl, 150 mM glycine and 20% [v/v] methanol) for 1.5 hr at 100V. Membranes were then blocked in blocking solution for an hr and incubated overnight at 4° C. with the following primary antibodies: mouse anti-HTT (1:200; Millipore-Sigma, MAB5374) and rabbit anti-β-actin (1:1000, Cell Signaling Technology, 4970S). Membranes were washed three times with TBS-T and incubated with either ready-to-use biotinylated goat Anti-Rabbit IgG (Abcam, ab64256) or ready-to-use biotinylated goat anti-mouse IgG (Abcam, ab64255) for 1 hr at RT. Membranes were then again washed three times in TBS-T and then incubated with a streptavidin-Alexa Fluor 700 conjugate (1:4000; Thermo Fisher Scientific, S21383) diluted in blocking solution (5% BSA in TBS-T) for 1 hr at RT. Membranes were washed three more times and visualized using an Odyssey Imaging System (LI-COR). Band intensity was quantitated using LI-COR Image Studio Software (LI-COR). Human mutant HTT protein was then normalized to the β-actin reference protein in each lane.

### RNA-Seq

Library construction was performed by the Roy J. Carver Biotechnology Center (University of Illinois). Purified DNase-treated RNAs were converted into individually barcoded polyadenylated mRNA-seq libraries using the TruSeq Stranded mRNA Sample Prep kit (Illumina). Libraries were then barcoded with Unique Dual Indexes (UDI's) to prevent index switching. Adaptor-ligated double-stranded cDNAs were then PCR amplified for 8 cycles with a Kapa HiFi polymerase (Roche). Final libraries were quantitated by Qubit (ThermoFisher) and the average cDNA fragment sizes were determined on a Fragment Analyzer. Libraries were diluted to 10 nM and quantitated by qPCR on a CFX Connect Real-Time qPCR system (Bio-Rad) to confirm accurate pooling of barcoded libraries and to maximize the number of clusters in the flowcell. Barcoded RNA-seq libraries were sequenced by a NovaSeq 6000 (Illumina). FastQ read files were generated and demultiplexed using the bcl2fastq v 2.20 Conversion Software (Illumina). The quality of the demultiplexed FastQ files was evaluated using FastQC. RNA-seq analysis was conducted by the High-Performance Biological Computing Core (University of Illinois). Briefly. Salmon3 version 1.2.0 was used to quasi-map reads to the transcriptome and to quantify the abundance of each transcript. Transcriptomes were indexed using the decoy-aware method in Salmon with the entire genome file and gene-level counts were estimated based on transcript-level counts using the “bias corrected counts without an offset” method from the tximport package.

### AAV Packaging

AAV vectors were packaged according to a previously established protocol (68). Briefly, 2×10⁷ HEK293T cells were seeded onto 15 cm cell culture plates in DMEM supplemented with 10% (v/v) FBS (ThermoFisher Scientific) and 1% (v/v) antibiotic-antimycotic (ThermoFisher Scientific). At 16 hr after seeding, cells were transfected with 15 ug of either pAAV-CAG-RfxCas13d-U6-NTG, pAAV-CAG-RfxCas13d-U6-SOD1 or pAAV-CAG-RfxCas13d-U6-HTT and 15 ug of AAV1 or AAV9 and 15 ug pHelper using 135 μL of polyethylamine (PEI) (1 μg/uL). Cells were gently harvested after 72 hr using a cell scraper, centrifuged at 4,000×g for 5 min at RT, and then resuspended in lysis buffer (50 mM Tris-HCl and 150 mM NaCl, pH 8.0). Using liquid nitrogen and a water bath set to 37° C., cells were freeze-thawed three times then incubated with 10 U of Benzonase (Sigma-Aldrich) per 1 mL of cell supernatant for 30 min at 37° C. Supernatant was then centrifuged at 18,500×g for 30 min at RT and the resulting lysate was overlayed on an iodixanol density gradient, which was centrifuged at 140,000×g for 2 hr at 18° C. After centrifugation, the virus was isolated by extraction, washed three times with 15 mL of PBS with 0.001% Tween 20 using an Ultra-15 centrifugal filter unit (Amicon) and concentrated to <150 μL. Viral genomic titer was determined by qPCR using iTaq Universal SYBR Green Supermix (Bio-Rad) and stored at 4° C.

### Injections

All animal procedures were approved by the Illinois Institutional Animal Care and Use Committee (IACUC) at the University of Illinois and conducted in accordance with the National Institutes of Health (NIH) Guide for Care and Use of Laboratory Animals. Treatment and control groups for all studies were sex-balanced and litter-matched.

Genotyped P60-P65 G93A-SOD1 mice bred from male G93A-SOD1 mice (B6SJL-Tg(SOD1*G93A)1Gur/J; Jackson Laboratory, Stock #002726) and female B6SJLF1/J mice (Jackson Laboratory, Stock #100012) were injected with 2×10¹¹ vg of AAV9-RfxCas13d-hSOD1 or AAV9-RfxCas13d-NTG in 10 mL PBS with 0.001% Tween-20 into the mouse lumbar subarachnoid space between the L5 and L6 vertebrae using a Hamilton syringe with a 29 gauge 1.5-inch needle. The crRNA used in the animal study corresponded to SOD1 crRNA 3 from our initial screen.

Genotyped P28 R6/2 mice bred from male R6/2 mice (B6CBA-Tg(HDexon1)62Gpb/3J; Jackson Laboratory, Stock #006494) and female B6CBAF 1/J mice (Jackson Laboratory, Stock #100011) were injected with a total of 6×10¹⁰ vg of AAV1-RfxCas13d-HTT (left hemisphere) and AAV1-RfxCas13d-NTG (right hemisphere) in 3 μL PBS with 0.001% Tween-20 at stereotaxic coordinates anterior-posterior (AP)=0.50 mm; medial-lateral (ML)=+1.65 mm; and dorsal-ventral (DV)=−3.5 mm, −3.0 mm, and −2.5 mm using a 25 μL syringe with a 30 gauge Point Style 4 needle with a 30° angle (Hamilton). Brain injections were performed using a drill and injection robot (NeuroStar). The crRNA used in this animal study corresponded to HTT crRNA 1 from our initial screen.

### Behavior

All behavior measurements were conducted by a blinded investigator. Beginning one week after injection, motor coordination of G93A-SOD1 mice was measured using a Rotamex-5 rotarod (Columbus Instruments). Mice were placed onto an apparatus programmed to accelerate from 4 to 40 rpm in 180 sec. The latency to fall was recorded and each session was composed of 3 trials.

Hindlimb strength was measured using a grip strength meter (Harvard Apparatus). Mice were scruffed and allowed to firmly latch onto a pull bar with their hindlimbs, then pulled in the opposite direction. The maximum force exerted prior to the release of the bar was recorded for each animal. Each session comprising of at least three measurements.

Weights of each mouse was recorded twice per week using an electronic scale. Disease onset and late-stage disease onset were then calculated as the day at which animals reached peak weight and lost 10% of their peak weight, respectively.

End-stage was determined as the point when the animal could no longer turn themselves over within 10 seconds of being placed on their back, lost more than 20% of their peak weight, or had complete paralysis. Mice were provided with wet, mashed food in their cages at the first sign of hindlimb paralysis and were monitored daily thereafter. All behavior measurements were normalized to the starting value at day 63.

### Immunohistochemistry

After transcardially perfusing mice with PBS, tissues were harvested and fixed overnight with 4% paraformaldehyde (PFA) in PBS at 4° C. then cut into 40 mm sagittal or coronal sections using a CM3050 S cryostat (Leica). Sections of spinal cord were then transferred to a 48-well plate, while sections of brain were transferred to a 24-well plate, then stored in cryoprotectant at −20° C. Sections washed three times with PBS and incubated with blocking solution (PBS with 10% [v/v] donkey serum [Abcam] and 1% Triton X-100) for 2 hr at RT and stained with primary antibodies in blocking solution for 72 hr at 4° C. Sections were then again washed three times with PBS and incubated with secondary antibodies in blocking solution for 2 hr at RT. Following incubation with the secondary antibodies, sections were washed three final times with PBS and then mounted onto slides using VECTASHIELD HardSet Antifade Mounting Medium (Vector Laboratories). Slides were imaged using a Leica TCS SP8 confocal microscope and a Zeiss Observer Z1 microscope (Beckman Institute Imaging Technology Microscopy Suite, University of Illinois). All image analyses were performed using ImageJ software.

The following primary antibodies were used: rabbit anti-hSOD1 (1:250; Cell Signaling Technology, 2770S), goat anti-ChAT (1:25; EMD Millipore, AB144P), goat anti-HA (1:250; GenScript, A00168), rabbit anti-HA (1:500; Cell Signaling Technology, 3724S), chicken anti-HA (1:500; Abcam, ab9111), rabbit anti-NeuN (1:500; Abcam, ab177487), rabbit anti-Ibal (1:500; Wako Pure Chemicals Industries, 019-19741), mouse anti-β3-tubulin (1:1,000; Sigma-Aldrich, T8578), chicken anti-GFAP (1:1,000; Abcam, ab4674), rabbit anti-DARPP-32 (1:100, Cell Signaling Technologies, 2306S) and mouse anti-HTT (1:50; Millipore-Sigma, MAB5374).

The following secondary antibodies were used: donkey anti-rabbit Cy3 (Jackson Fluor 488 (Jackson ImmunoResearch, 711-165-152), donkey anti-rabbit Alexa F ImmunoResearch, 711-545-152), donkey anti-goat Cy3 (Jackson ImmunoResearch, 705-165-147), donkey anti-goat Alexa Fluor 488 (Thermo Fisher Scientific, A-11055), donkey anti-goat Alexa Fluor 647 (Jackson ImmunoResearch, 705-605-147), donkey anti-mouse Alexa Fluor 488 (Jackson ImmunoResearch, 715-545-150), donkey anti-chicken Alexa Fluor 647 (Jackson ImmunoResearch, 703-605-155), donkey anti-chicken Cy3 (Jackson ImmunoResearch, 703-165-155), donkey anti-rat Cy3 (Jackson ImmunoResearch, 712-165-153), and donkey anti-mouse Alexa Fluor 488 (Jackson ImmunoResearch, 715-545-150).

Inclusion area was quantified by first highlighting an area by ImageJ. A pixel intensity threshold was then applied to identify regions covered by inclusions and the area of the respective region was analyzed by the measure function in ImageJ. The measured area was then normalized to the total area initially highlighted to derive the percentage of the area occupied by immunoreactive inclusions. All measurements were performed by a blinded investigator.

### Statistical Analysis

Statistical analysis was performed using GraphPad Prism 8. mRNA, protein, disease onset and late-stage disease onset were compared using an unpaired one-way t-test. Survival was analyzed by Kaplan-Meier analyses using the Mantel-Cox test. Rotarod and grip strength were analyzed using a two-way ANOVA followed by a Bonferroni post-hoc test. Weight loss and hind-limb grip strength were analyzed using a linear regression analysis. Reactive inclusion data were compared using a one-tailed unpaired t-test.

### REFERENCES

1. M. Jinek, K. Chylinski, I. Fonfara, M. Hauer, J. A. Doudna, E. Charpentier, A programmable dual-RNA-guided DNA endonuclease in adaptive bacterial immunity. Science 337, 816 (2012).
2. L. Cong, F. A. Ran, D. Cox, S. Lin, R. Barretto, N. Habib, P. D. Hsu, X. Wu, W. Jiang, L. A. Marraffini, F. Zhang, Multiplex genome engineering using CRISPR/Cas systems. Science 339, 819 (2013).
3. P. Mali, L. Yang, K. M. Esvelt, J. Aach, M. Guell, J. E. DiCarlo, J. E. Norville, G. M. Church, RNA-guided human genome engineering via Cas9. Science 339, 823 (2013).
4. O. O. Abudayyeh, J. S. Gootenberg, S. Konermann, J. Joung, I. M. Slaymaker, D. B. T. Cox, S. Shmakov, K. S. Makarova, E. Semenova, L. Minakhin, K. Severinov, A. Regev, E. S. Lander, E. V. Koonin, F. Zhang, C2c2 is a single-component programmable RNA-guided RNA-targeting CRISPR effector. Science 353, aaf5573 (2016).
5. A. A. Smargon, D. B. T. Cox, N. K. Pyzocha, K. Zheng, I. M. Slaymaker, J. S. Gootenberg, O. A. Abudayyeh, P. Essletzbichler, S. Shmakov, K. S. Makarova, E. V. Koonin, F. Zhang, Cas13b Is a type VI-B CRISPR-associated RNA-guided RNase differentially regulated by accessory proteins Csx27 and Csx28. Mol. Cell 65, 618-630.e617 (2017).
6. S. Konermann, P. Lotfy, N. J. Brideau, J. Oki, M. N. Shokhirev, P. D. Hsu, Transcriptome Engineering with RNA-Targeting Type VI-D CRISPR Effectors. Cell 173, 665-676.e614 (2018).
7. W. X. Yan, S. Chong, H. Zhang, K. S. Makarova, E. V. Koonin, D. R. Cheng, D. A. Scott, Cas13d Is a compact RNA-Targeting type VI CRISPR effector positively modulated by a WYL-domain-containing accessory protein. Mol. Cell 70, 327-339.e325 (2018).
8. A. East-Seletsky, M. R. O'Connell, S. C. Knight, D. Burstein, J. H. Cate, R. Tjian, J. A. Doudna, Two distinct RNase activities of CRISPR-C2c2 enable guide-RNA processing and RNA detection. Nature 538, 270-273 (2016).
9. O. O. Abudayyeh, J. S. Gootenberg, P. Essletzbichler, S. Han, J. Joung, J. J. Belanto, V. Verdine, D. B. T. Cox, M. J. Kellner, A. Regev, E. S. Lander, D. F. Voytas, A. Y. Ting, F. Zhang, RNA targeting with CRISPR-Cas13. Nature 550, 280-284 (2017).
10. D. B. T. Cox, J. S. Gootenberg, O. O. Abudayyeh, B. Franklin, M. J. Kellner, J. Joung, F. Zhang, RNA editing with CRISPR-Cas13. Science 358, 1019-1027 (2017).
11. C. Xu, Y. Zhou, Q. Xiao, B. He, G. Geng, Z. Wang, B. Cao, X. Dong, W. Bai, Y. Wang, X. Wang, D. Zhou, T. Yuan, X. Huo, J. Lai, H. Yang, Programmable RNA editing with compact CRISPR-Cas13 systems from uncultivated microbes. Nat. Methods 18, 499-506 (2021).
12. H.-H. Wessels, A. Mendez-Mancilla, X. Guo, M. Legut, L. Damloski, N. E. Sanjana, Massively parallel Cas13 screens reveal principles for guide RNA design. Nat. Biotechnol. 38, 722-727 (2020).
13. N. Huynh, N. Depner, R. Larson, K. King-Jones, A versatile toolkit for CRISPR-Cas13-based RNA manipulation in Drosophila. Genome Biol. 21, 279 (2020).
14. B. He, W. Peng, J. Huang, H. Zhang, Y. Zhou, X. Yang, J. Liu, Z. Li, C. Xu, M. Xue, H. Yang, P. Huang, Modulation of metabolic functions through Cas13d-mediated gene knockdown in liver. Protein Cell 11, 518-524 (2020).
15. H. Zhou, J. Su, X. Hu, C. Zhou, H. Li, Z. Chen, Q. Xiao, B. Wang, W. Wu, Y. Sun, Y. Zhou, C. Tang, F. Liu, L. Wang, C. Feng, M. Liu, S. Li, Y. Zhang, H. Xu, H. Yao, L. Shi, H. Yang, Glia-to-neuron conversion by CRISPR-CasRx alleviates symptoms of neurological disease in mice. Cell 181, 590-603.e516 (2020).
16. C. Zhou, X. Hu, C. Tang, W. Liu, S. Wang, Y. Zhou, Q. Zhao, Q. Bo, L. Shi, X. Sun, H. Zhou, H. Yang, CasRx-mediated RNA targeting prevents choroidal neovascularization in a mouse model of age-related macular degeneration. Natl. Sci. Rev. 7, 835-837 (2020).
17. D. R. Rosen, T. Siddique, D. Patterson, D. A. Figlewicz, P. Sapp, A. Hentati, D. Donaldson, J. Goto, J. P. O'Regan, H.-X. Deng, Z. Rahmani, A. Krizus, D. McKenna-Yasek, A. Cayabyab, S. M. Gaston, R. Berger, R. E. Tanzi, J. J. Halperin, B. Herzfeldt, R. Van den Bergh, W.-Y. Hung, T. Bird, G. Deng, D. W. Mulder, C. Smyth, N. G. Laing, E. Soriano, M. A. Pericak-Vance, J. Haines, G. A. Rouleau, J. S. Gusella, H. R. Horvitz, R. H. Brown, Mutations in Cu/Zn superoxide dismutase gene are associated with familial amyotrophic lateral sclerosis. Nature 362, 59-62 (1993).
18. R. H. Brown, A. Al-Chalabi, Amyotrophic lateral sclerosis. N. Eng. J. Med. 377, 162-172 (2017).
19. C. Mueller, J. D. Berry, D. M. McKenna-Yasek, G. Gernoux, M. A. Owegi, L. M. Pothier, C. L. Douthwright, D. Gelevski, S. D. Luppino, M. Blackwood, N. S. Wightman, D. H. Oakley, M. P. Frosch, T. R. Flotte, M. E. Cudkowicz, R. H. Brown, SOD1 Suppression with Adeno-Associated Virus and MicroRNA in Familial ALS. N. Eng. J. Med. 383, 151-158 (2020).
20. T. Miller, M. Cudkowicz, P. J. Shaw, P. M. Andersen, N. Atassi, R. C. Bucelli, A. Genge, J. Glass, S. Ladha, A. L. Ludolph, N. J. Maragakis, C. J. McDermott, A. Pestronk, J. Ravits, F. Salachas, R. Trudell, P. Van Damme, L. Zinman, C. F. Bennett, R. Lane, A. Sandrock, H. Runz, D. Graham, H. Houshyar, A. McCampbell, I. Nestorov, I. Chang, M. McNeill, L. Fanning, S. Fradette, T. A. Ferguson, Phase 1-2 Trial of Antisense Oligonucleotide Tofersen for SOD1 ALS. N. Eng. J. Med. 383, 109-119 (2020).
21. O. Hardiman, Major advances in amyotrophic lateral sclerosis in 2020. Lancet Neurol. 20, 14-15 (2021).
22. E. L. Blanchard, D. Vanover, S. S. Bawage, P. M. Tiwari, L. Rotolo, J. Beyersdorf, H. E. Peck, N. C. Bruno, R. Hincapie, F. Michel, J. Murray, H. Sadhwani, B. Vanderheyden, M. G. Finn, M. A. Brinton, E. R. Lafontaine, R. J. Hogan, C. Zurla, P. J. Santangelo, Treatment of influenza and SARS-CoV-2 infections via mRNA-encoded Cas13a in rodents. Nat. Biotechnol. 39, 717-726 (2021).
23. S. Boillée, K. Yamanaka, C. S. Lobsiger, N. G. Copeland, N. A. Jenkins, G. Kassiotis, G. Kollias, D. W. Cleveland, Onset and progression in inherited ALS determined by motor neurons and microglia. Science 312, 1389-1392 (2006).
24. K. D. Foust, D. L. Salazar, S. Likhite, L. Ferraiuolo, D. Ditsworth, H. Ilieva, K. Meyer, L. Schmelzer, L. Braun, D. W. Cleveland, B. K. Kaspar, Therapeutic AAV9-mediated suppression of mutant SOD1 slows disease progression and extends survival in models of inherited ALS. Mol. Ther. 21, 2148-2159 (2013).
25. G. M. Thomsen, G. Gowing, J. Latter, M. Chen, J. P. Vit, K. Staggenborg, P. Avalos, M. Alkaslasi, L. Ferraiuolo, S. Likhite, B. K. Kaspar, C. N. Svendsen, Delayed disease onset and extended survival in the SOD1G93A rat model of amyotrophic lateral sclerosis after suppression of mutant SOD1 in the motor cortex. J. Neurosci. 34, 15587-15600 (2014).
26. T. Gaj, D. S. Ojala, F. K. Ekman, L. C. Byrne, P. Limsirichai, D. V. Schaller, in vivo genome editing improves motor function and extends survival in a mouse model of ALS. Sci. Adv. 3, eaar3952 (2017).
27. M. Nagai, D. B. Re, T. Nagata, A. Chalazonitis, T. M. Jessell, H. Wichterle, S. Przedborski, Astrocytes expressing ALS-linked mutated SOD1 release factors selectively toxic to motor neurons. Nat. Neurosci. 10, 615-622 (2007).
28. K. Yamanaka, S. J. Chun, S. Boillee, N. Fujimori-Tonou, H. Yamashita, D. H. Gutmann, R. Takahashi, H. Misawa, D. W. Cleveland, Astrocytes as determinants of disease progression in inherited amyotrophic lateral sclerosis. Nat. Neurosci. 11, 251 (2008).
29. M. C. Marchetto, A. R. Muotri, Y. Mu, A. M. Smith, G. G. Cezar, F. H. Gage, Non-cell-autonomous effect of human SOD1 G37R astrocytes on motor neurons derived from human embryonic stem cells. Cell Stem Cell 3, 649-657 (2008).
30. A. M. Haidet-Phillips, M. E. Hester, C. J. Miranda, K. Meyer, L. Braun, A. Frakes, S. Song, S. Likhite, M. J. Murtha, K. D. Foust, M. Rao, A. Eagle, A. Kammesheidt, A. Christensen, J. R. Mendell, A. H. M. Burghes, B. K. Kaspar, Astrocytes from familial and sporadic ALS patients are toxic to motor neurons. Nat. Biotechnol. 29, 824 (2011).
31. C. K. W. Lim, M. Gapinske, A. K. Brooks, W. S. Woods, J. E. Powell, M. A. Zeballos C, J. Winter, P. Perez-Pinera, T. Gaj, Treatment of a Mouse Model of ALS by In Vivo Base Editing. Mol. Ther. 28, 1177-1189 (2020).
32. H. Wang, B. Yang, L. Qiu, C. Yang, J. Kramer, Q. Su, Y. Guo, R. H. Brown, Jr., G. Gao, Z. Xu, Widespread spinal cord transduction by intrathecal injection of rAAV delivers efficacious RNAi therapy for amyotrophic lateral sclerosis. Hum. Mol. Genet. 23, 668-681 (2014).
33. K. Bey, C. Ciron, L. Dubreil, J. Deniaud, M. Ledevin, J. Cristini, V. Blouin, P. Aubourg, M. A. Colle, Efficient CNS targeting in adult mice by intrathecal infusion of single-stranded AAV9-GFP for gene therapy of neurological disorders. Gene Ther. 24, 325-332 (2017).
34. Y. Gong, A. Berenson, F. Laheji, G. Gao, D. Wang, C. Ng, A. Volak, R. Kok, V. Kreouzis, I. M. Dijkstra, S. Kemp, C. A. Maguire, F. Eichler, Intrathecal adeno-associated viral vector-mediated gene delivery for adrenomyeloneuropathy. Hum. Gene Ther. 30, 544-555 (2019).
35. M. Gurney, H. Pu, A. Chiu, M. Dal Canto, C. Polchow, D. Alexander, J. Caliendo, A. Hentati, Y. Kwon, H. Deng, a. et, Motor neuron degeneration in mice that express a human Cu,Zn superoxide dismutase mutation. Science 264, 1772-1775 (1994).
36. S. Vinsant, C. Mansfield, R. Jimenez-Moreno, V. Del Gaizo Moore, M. Yoshikawa, T. G. Hampton, D. Prevette, J. Caress, R. W. Oppenheim, C. Milligan, Characterization of early pathogenesis in the SOD1(G93A) mouse model of ALS: part II, results and discussion. Brain Behav. 3, 431-457 (2013).
37. Y. Gong, A. Berenson, F. Laheji, G. Gao, D. Wang, C. Ng, A. Volak, R. Kok, V. Kreouzis, I. M. Dijkstra, S. Kemp, C. A. Maguire, F. Eichler, Intrathecal Adeno-Associated Viral Vector-Mediated Gene Delivery for Adrenomyeloneuropathy. Hum. Gene Ther. 30, 544-555 (2019).
38. T. Gaj, D. S. Ojala, F. K. Ekman, L. C. Byrne, P. Limsirichai, D. V. Schaffer, In vivo genome editing improves motor function and extends survival in a mouse model of ALS. Science Adv. 3, eaar3952 (2017).
39. E. Dirren, J. Aebischer, C. Rochat, C. Towne, B. L. Schneider, P. Aebischer, SOD1 silencing in motoneurons or glia rescues neuromuscular function in ALS mice. Ann. Clin. Transl. Neurol. 2, 167-184 (2015).
40. L. Wang, D. H. Gutmann, R. P. Roos, Astrocyte loss of mutant SOD1 delays ALS disease onset and progression in G85R transgenic mice. Hum. Mol. Genet. 20, 286-293 (2011).
41. F. Saudou, S. Humbert, The Biology of Huntingtin. Neuron 89, 910-926 (2016).
42. S. J. Tabrizi, M. D. Flower, C. A. Ross, E. J. Wild, Huntington disease: new insights into molecular pathogenesis and therapeutic opportunities. Nat. Rev. Neurosci. 16, 529-546 (2020).
43. H. B. Kordasiewicz, L. M. Stanek, E. V. Wancewicz, C. Mazur, M. M. McAlonis, K. A. Pytel, J. W. Artates, A. Weiss, S. H. Cheng, L. S. Shihabuddin, G. Hung, C. F. Bennett, D. W. Cleveland, Sustained therapeutic reversal of Huntington's disease by transient repression of huntingtin synthesis. Neuron 74, 1031-1044 (2012).
44. S. J. Tabrizi, B. R. Leavitt, G. B. Landwehrmeyer, E. J. Wild, U. Salt, K. A. Barker, N. F. Blair, D. Craufurd, J. Priller, H. Rickards, A. Rosser, H. B. Kordasiewicz, C. Czech, E. E. Swayze, D. A. Norris, T. Baumann, I. Gerlach, S. A. Schobel, E. Paz, A. V. Smith, C. F. Bennett, R. M. Lane, Targeting huntingtin expression in patients with Huntington's disease. N. Eng. J. Med. 380, 2307-2316 (2019).
45. C. J. Maynard, C. Bottcher, Z. Ortega, R. Smith, B. I. Florea, M. Diaz-Hernindez, P. Brundin, H. S. Overkleeft, J. Y. Li, J. J. Lucas, N. P. Dantuma, Accumulation of ubiquitin conjugates in a polyglutamine disease model occurs without global ubiquitin/proteasome system impairment. Proc Natl Acad Sci USA 106, 13986-13991 (2009).
46. A. B. Buchman, D. J. Brogan, R. Sun, T. Yang, P. D. Hsu, O. S. Akbari, Programmable RNA rargeting using CasRx in flies. CRISPR J. 3, 164-176 (2020).
47. B. W. Hounkpe, F. Chenou, F. de Lima, Erich V. De Paula, HRT Atlas v 1.0 database: redefining human and mouse housekeeping genes and candidate reference transcripts by mining massive RNA-seq datasets. Nucleic Acids Res. 49, D947-D955 (2021).
48. L. Mangiarini, K. Sathasivam, M. Seller, B. Cozens, A. Harper, C. Hetherington, M. Lawton, Y. Trottier, H. Lehrach, S. W. Davies, G. P. Bates, Exon 1 of the HD gene with an expanded CAG repeat is sufficient to cause a progressive neurological phenotype in transgenic mice. Cell 87, 493-506 (1996).
49. F. K. Ekman, D. S. Ojala, M. M. Adil, P. A. Lopez, D. V. Schaffer, T. Gaj, CRISPR-Cas9-mediated genome editing increases lifespan and improves motor deficits in a Huntington's disease mouse model. Mol. Ther. Nucleic Acids 17, 829-839 (2019).
50. J. Y. Li, N. Popovic, P. Brundin, The use of the R6 transgenic mouse models of Huntington's disease in attempts to develop novel therapeutic strategies. NeuroRx 2, 447-464 (2005).
51. T. Liu, W. Im, I. Mook-Jung, M. Kim, MicroRNA-124 slows down the progression of Huntington's disease by promoting neurogenesis in the striatum. Neural Regen. Res. 10, 786-791 (2015).
52. C. F. Bennett, A. R. Kramer, D. W. Cleveland, Antisense oligonucleotide herapies for neurodegenerative diseases. Annu. Rev. Neurosci. 42, 385-406 (2019).
53. A. C. Silva, D. D. Lobo, I. M. Martins, S. M. Lopes, C. Henriques, S. P. Duarte, J.-C. Dodart, R. J. Nobre, L. Pereira de Almeida, Antisense oligonucleotide therapeutics in neurodegenerative diseases: the case of polyglutamine disorders. Brain 143, 407-429 (2020).
54. R. Martier, P. Konstantinova, Gene therapy for neurodegenerative diseases: slowing down the ticking clock. Front. Neurosci. 14, (2020).
55. R. Ghosh, S. J. Tabrizi, Gene suppression approaches to neurodegeneration. Alzheimer's Res. Ther. 9, 82 (2017).
56. A. Vallés, M. M. Evers, A. Stam, M. Sogorb-Gonzalez, C. Brouwers, C. Vendrell-Tornero, S. Acar-Broekmans, L. Paerels, J. Klima, B. Bohuslavova, R. Pintauro, V. Fodale, A. Bresciani, R. Liscak, D. Urgosik, Z. Starek, M. Crha, B. Blits, H. Petry, Z. Ellederova, J. Motlik, S. van Deventer, P. Konstantinova, Widespread and sustained target engagement in Huntington's disease minipigs upon intrastriatal microRNA-based gene therapy. Sci. Transl. Med. 13, eabb8920 (2021).
57. M. M. Janas, M. K. Schlegel, C. E. Harbison, V. O. Yilmaz, Y. Jiang, R. Parmar, I. Zlatev, A. Castoreno, H. Xu, S. Shulga-Morskaya, K. G. Rajeev, M. Manoharan, N. D. Keirstead, M. A. Maier, V. Jadhav, Selection of GalNAc-conjugated siRNAs with limited off-target-driven rat hepatotoxicity. Nat. Commun. 9, 723 (2018).
58. F. Borel, G. Gernoux, B. Cardozo, J. P. Metterville, G. C. Toro Cabreja, L. Song, Q. Su, G. P. Gao, M. K. Elmallah, R. H. Brown, Jr., C. Mueller, Therapeutic rAAVrh10 mediated SOD1 silencing in adult SOD1(G93A) mice and nonhuman primates. Hum. Gene Ther. 27, 19-31 (2016).
59. A. McCampbell, T. Cole, A. J. Wegener, G. S. Tomassy, A. Setnicka, B. J. Farley, K. M. Schoch, M. L. Hoye, M. Shabsovich, L. Sun, Y. Luo, M. Zhang, N. Comfort, B. Wang, J. Amacker, S. Thankamony, D. W. Salzman, M. Cudkowicz, D. L. Graham, C. F. Bennett, H. B. Kordasiewicz, E. E. Swayze, T. M. Miller, Antisense oligonucleotides extend survival and reverse decrement in muscle response in ALS models. J. Clin. Invest. 128, 3558-3567 (2018).
60. C. Raoul, T. Abbas-Terki, J.-C. Bensadoun, S. Guillot, G. Haase, J. Szulc, C. E. Henderson, P. Aebischer, Lentiviral-mediated silencing of SOD1 through RNA interference retards disease onset and progression in a mouse model of ALS. Nat. Med. 11, 423-428 (2005).
61. M. DiFiglia, M. Sena-Esteves, K. Chase, E. Sapp, E. Pfister, M. Sass, J. Yoder, P. Reeves, R. K. Pandey, K. G. Rajeev, M. Manoharan, D. W. Y. Sah, P. D. Zamore, N. Aronin, Therapeutic silencing of mutant huntingtin with siRNA attenuates striatal and cortical neuropathology and behavioral deficits. Proc. Natl. Acad. Sci. U.S.A. 104, 17204 (2007).
62. R. L. Boudreau, J. L. McBride, I. Martins, S. Shen, Y. Xing, B. J. Carter, B. L. Davidson, Nonallele-specific silencing of mutant and wild-type huntingtin demonstrates therapeutic efficacy in Huntington's disease mice. Mol. Ther. 17, 1053-1063 (2009).
63. S. Q. Harper, P. D. Staber, X. He, S. L. Eliason, I. H. Martins, Q. Mao, L. Yang, R. M. Kotin, H. L. Paulson, B. L. Davidson, RNA interference improves motor and neuropathological abnormalities in a Huntington's disease mouse model. Proc. Natl. Acad. Sci. U.S.A. 102, 5820-5825 (2005).
64. H.-H. Wessels, A. Mendez-Mancilla, X. Guo, M. Legut, Z. Daniloski, N. E. Sanjana, Massively parallel Cas13 screens reveal principles for guide RNA design. Nature Biotechnology 38, 722-727 (2020).
65. C. T. Charlesworth, P. S. Deshpande, D. P. Dever, J. Camarena, V. T. Lemgart, M. K. Cromer, C. A. Vakulskas, M. A. Collingwood, L. Zhang, N. M. Bode, M. A. Behlke, B. Dejene, B. Cieniewicz, R. Romano, B. J. Lesch, N. Gomez-Ospina, S. Mantri, M. Pavel-Dinu, K. I. Weinberg, M. H. Porteus, Identification of preexisting adaptive immunity to Cas9 proteins in humans. Nat. Med. 25, 249-254 (2019).
66. W. L. Chew, M. Tabebordbar, J. K. W. Cheng, P. Mali, E. Y. Wu, A. H. M. Ng, K. Zhu, A. J. Wagers, G. M. Church, A multifunctional AAV-CRISPR-Cas9 and its host response. Nat. Methods 13, 868-874 (2016).
67. S. R. Ferdosi, R. Ewaisha, F. Moghadam, S. Krishna, J. G. Park, M. R. Ebrahimkhani, S. Kiani, K. S. Anderson, Multifunctional CRISPR-Cas9 with engineered immunosilenced human T cell epitopes. Nat. Commun. 10, 1842 (2019).
68. T. Gaj, D. V. Schaffer, Adeno-associated virus-mediated delivery of CRISPR-Cas systems for genome engineering in mammalian cells. Cold Spring Harb. Protoc. 2016, pdb.prot086868 (2016).

We claim:

1. A recombinant adeno-associated virus (rAAV) vector comprising in 5′ to 3′ direction:
(a) a first AAV inverted terminal repeat (ITR) sequence;
(b) a crRNA sequence having homology to a superoxide dismutase 1 (SOD1) gene or a huntingtin (HTT) gene; and
(c) a second AAV ITR sequence.

2. The rAAV vector of claim 1, further comprising between the first AAV ITR and the second AAV ITR sequence:
(a) a promoter sequence;
(b) a nucleic acid molecule encoding a Cas13 polypeptide; and
(c) a polyA sequence.

3. The rAAV vector of any of the preceding claims, wherein the rAAV vector further comprises one or more nuclear localization signals (NLSs).

4. The rAAV vector of claim 3, wherein the one or more NLSs occur 5′ to the nucleic acid molecule encoding the Cas13 polypeptide, 3′ to the nucleic acid molecule encoding the Cas13 polypeptide, or both.

5. The rAAV vector of any of claims 2-4, wherein the promoter is a cytomegalovirus early enhancer/chicken β-actin (CAG) promoter.

6. The rAAV vector of any of the preceding claims, wherein the vector further comprises one or more human influenza hemagglutinin (HA) epitope tags.

7. The rAAV vector of claims 2-6, wherein the polyA sequence is a bovine growth hormone (BGH) polyA sequence.

8. The rAAV vector of any of the preceding claims, wherein the crRNA sequence comprises one or more of SEQ ID NOs:58-71.

9. The rAAV vector of claim 8, wherein the crRNA further comprises a Cas13-specific direct repeat region.

10. The rAAV vector of claim 9, where the Cas13-specific direct repeat region comprises the sequence set forth in SEQ ID NO:90.

11. The rAAV vector of any of the preceding claims, wherein the Cas13 polypeptide is a Cas13d polypeptide.

12. The rAAV vector of any of the preceding claims, wherein the Cas13 polypeptide is a Ruminococcus flavefaciens Cas13d (RfxCas13d) polypeptide.

13. The rAAV vector of any of the preceding claims, wherein the Cas13 polypeptide is a catalytically deactivated Cas13 (dCas13) polypeptide.

14. The rAAV vector of claim 13, wherein the dCas13 polypeptide is a dCas13d polypeptide.

15. The rAAV vector of any of the preceding claims, further comprising a nucleic acid molecule encoding an AAV capsid protein.

16. The rAAV vector of claim 15, wherein the AAV capsid protein is an AAV1 capsid protein, an AAV2 capsid protein, an AAV4 capsid protein, an AAV5 capsid protein, an AAV6 capsid protein, an AAV7 capsid protein, an AAV8 capsid protein, an AAV9 capsid protein, an AAV10 capsid protein, an AAV11 capsid protein, an AAV12 capsid protein, an AAV13 capsid protein, an AAVPHP.B capsid protein, an AAVrh74 capsid protein or an AAVrh.10 capsid protein.

17. The rAAV vector of claim 16, wherein the AAV capsid protein is an AAV9 capsid protein.

18. A pharmaceutical composition comprising:
the rAAV vector of any one of the preceding claims and at least one pharmaceutically acceptable excipient and/or additive.

19. A method for treating a subject having a disease and/or disorder involving an SOD1 gene or an HTT gene, the method comprising administering to the subject at least one therapeutically effective amount of the rAAV vector of any one of claims 1-17 or the pharmaceutical composition of claim 18.

20. The method of claim 19, wherein the disease and/or disorder involving the SOD1 gene is amyotrophic lateral sclerosis (ALS), familial amyotrophic lateral sclerosis, or Parkinson's disease, and the disease and/or disorder involving the HTT gene is Huntington's disease (HD).

21. The method of any of claims 18-20, wherein the rAAV viral vector or the pharmaceutical composition is administered to the subject at a dose ranging from about 10¹¹ to about 10¹⁸ viral vector particles.

22. The method of any of claims 18-20, wherein the rAAV viral vector or the pharmaceutical composition is administered to the subject at a dose ranging from about 10¹³ to about 10¹⁶ viral vector particles.

23. The method of any of claims 18-20, wherein the rAAV viral vector or the pharmaceutical composition is administered to the subject intravenously, intrathecally, intrastriatally, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally or intranerve.

24. The method of any of claims 18-20, wherein the rAAV viral vector or pharmaceutical composition is administered intrathecally or intrastriatally.

25. The rAAV vector of any one of claim 1-17 or 19-24 or the pharmaceutical composition of claim 18 for use in treating a disease and/or disorder involving an SOD1 or HTT gene in a subject in need thereof.

26. Use of an rAAV viral vector of any one of claim 1-17 or 19-24 or the pharmaceutical composition of claim 18.

27. The use of claim 26, wherein the disease and/or disorder involving the SOD1 gene is amyotrophic lateral sclerosis (ALS), familial amyotrophic lateral sclerosis, and Parkinson's disease and the disease and/or disorder involving the HTT gene is HD.

28. The use of claim 26, wherein the rAAV viral vector or the pharmaceutical composition is for administration to the subject at a dose ranging from about 10¹¹ to about 10¹⁸ viral vector particles.

29. The use of claim 26, wherein the rAAV viral vector or the pharmaceutical composition is for administration to the subject at a dose ranging from about 10¹³ to about 10¹⁶ viral vector particles.

30. The use of claim 26, wherein the rAAV viral vector or the pharmaceutical composition is for administration to the subject intravenously, intrathecally, intrastriatally, intracerebrally, intraventricularly, intranasally, intratracheally, intra-aurally, intra-ocularly, or peri-ocularly, orally, rectally, transmucosally, inhalationally, transdermally, parenterally, subcutaneously, intradermally, intramuscularly, intracisternally, intranervally, intrapleurally, topically, intralymphatically, intracisternally, or intranerve.

31. The use of claim 26, wherein the rAAV viral vector or pharmaceutical composition is for administration intrathecally or intrastriatally.

32. A method of reducing an amount of mRNA encoding SOD1 or HTT in a cell comprising delivering the rAAV vector of any one of claim 1-17 or 19-24 to the cell.

33. An isolated crRNA comprising one or more of the nucleotide sequences set forth in SEQ ID NOs: 58-71.

34. The isolated crRNA of claim 33 further comprising a Cas13-specific direct repeat region.

35. The isolated crRNA of claim 34, wherein the Cas13-specific direct repeat region is set forth in SEQ ID NO:90.

36. The isolated crRNA of claim 33 further comprising a promoter sequence.

37. The (rAAV) vector of claim 1, wherein a promoter is associated with the crRNA sequence having homology to a superoxide dismutase 1 (SOD1) gene or a huntingtin (HTT) gene, such that the promoter drives expression of the crRNA sequence.


SEQUENCE LISTING (92 sequences)

SEQ ID NO: 1 - protein, 7 aa, synthetic construct
  REGION 1..7: note = V40 large T antigen NLS
PKKKRRV

SEQ ID NO: 2 - protein, 16 aa, synthetic construct
  REGION 1..16: note = nucleoplasmin NLS
KRPAATKKAGQAKKKK

SEQ ID NO: 3 - RNA, 36 nt, synthetic construct
  misc_feature 1..36: note = Cas13-specific direct repeat
  source 1..36: mol_type = other RNA
caagtaaacccctaccaactggtcggggtttgaaac

SEQ ID NO: 4 - RNA, 34 nt, synthetic construct
  modified_base 6..7: mod_base = OTHER; note = thymine
  modified_base 9: mod_base = OTHER; note = thymine
  modified_base 12: mod_base = OTHER; note = thymine
  modified_base 18..19: mod_base = OTHER; note = thymine
  modified_base 22: mod_base = OTHER; note = thymine
  modified_base 25: mod_base = OTHER; note = thymine
  modified_base 30: mod_base = OTHER; note = thymine
  misc_feature 1..34: note = hSOD1 crRNA 1 Fwd primer
  source 1..34: mol_type = other RNA
aaaccttctgctcgaaattgatgatgccctgcac

SEQ ID NO: 5 - RNA, 34 nt, synthetic construct
  modified_base 6: mod_base = OTHER; note = thymine
  modified_base 15: mod_base = OTHER; note = thymine
  modified_base 18: mod_base = OTHER; note = thymine
  modified_base 22..24: mod_base = OTHER; note = thymine
  misc_feature 1..34: note = hSOD1 crRNA 1 Rev primer
  source 1..34: mol_type = other RNA
aaaagtgcagggcatcatcaatttcgagcagaag

SEQ ID NO: 6 - RNA, 34 nt, synthetic construct
  modified_base 5: mod_base = OTHER; note = thymine
  modified_base 15..16: mod_base = OTHER; note = thymine
  modified_base 20: mod_base = OTHER; note = thymine
  modified_base 23: mod_base = OTHER; note = thymine
  modified_base 27..28: mod_base = OTHER; note = thymine
  modified_base 31..33: mod_base = OTHER; note = thymine
  misc_feature 1..34: note = hSOD1 crRNA 2 Fwd primer
  source 1..34: mol_type = other RNA
aaactccccacaccttcactggtccattactttc

SEQ ID NO: 7 - RNA, 34 nt, synthetic construct
  modified_base 10: mod_base = OTHER; note = thymine
  modified_base 13: mod_base = OTHER; note = thymine
  modified_base 21: mod_base = OTHER; note = thymine
  modified_base 27: mod_base = OTHER; note = thymine
  modified_base 29: mod_base = OTHER; note = thymine
  misc_feature 1..34: note = hSOD1 crRNA 2 Rev primer
  source 1..34: mol_type = other RNA
aaaagaaagtaatggaccagtgaaggtgtgggga

SEQ ID NO: 8 - RNA, 34 nt, synthetic construct
  modified_base 5: mod_base = OTHER; note = thymine
  modified_base 9: mod_base = OTHER; note = thymine
  modified_base 12: mod_base = OTHER; note = thymine
  modified_base 16..17: mod_base = OTHER; note = thymine
  modified_base 20..22: mod_base = OTHER; note = thymine
  modified_base 25..26: mod_base = OTHER; note = thymine
  modified_base 28: mod_base = OTHER; note = thymine
  modified_base 31: mod_base = OTHER; note = thymine
  misc_feature 1..34: note = hSOD1 crRNA 3 Fwd primer
  source 1..34: mol_type = other RNA
aaactcactggtccattactttccttctgctcga

SEQ ID NO: 9 - RNA, 34 nt, synthetic construct
  modified_base 5: mod_base = OTHER; note = thymine
  modified_base 21: mod_base = OTHER; note = thymine
  modified_base 24: mod_base = OTHER; note = thymine
  modified_base 32: mod_base = OTHER; note = thymine
  misc_feature 1..34: note = hSOD1 crRNA 3 Rev primer
  source 1..34: mol_type = other RNA
aaaatcgagcagaaggaaagtaatggaccagtga

SEQ ID NO: 10 - RNA, 34 nt, synthetic construct
  modified_base 6..7: mod_base = OTHER; note = thymine
  modified_base 9: mod_base = OTHER; note = thymine
  modified_base 11: mod_base = OTHER; note = thymine
  modified_base 18: mod_base = OTHER; note = thymine
  modified_base 21: mod_base = OTHER; note = thymine
  modified_base 27: mod_base = OTHER; note = thymine
  modified_base 32: mod_base = OTHER; note = thymine
  misc_feature 1..34: note = hSOD1 crRNA 4 Fwd primer
  source 1..34: mol_type = other RNA
aaacattatctccaaactcatgaacatggaatcc

SEQ ID NO: 11 - RNA, 34 nt, synthetic construct
  modified_base 8..9: mod_base = OTHER; note = thymine
  modified_base 13: mod_base = OTHER; note = thymine
  modified_base 15..16: mod_base = OTHER; note = thymine
  modified_base 19: mod_base = OTHER; note = thymine
  modified_base 23..25: mod_base = OTHER; note = thymine
  modified_base 31: mod_base = OTHER; note = thymine
  modified_base 34: mod_base = OTHER; note = thymine
  misc_feature 1..34: note = hSOD1 crRNA 4 Rev primer
  source 1..34: mol_type = other RNA
aaaaggattccatgttcatgagtttggagataat

SEQ ID NO: 12 - RNA, 34 nt, synthetic construct
  modified_base 7: mod_base = OTHER; note = thymine
  modified_base 10..12: mod_base = OTHER; note = thymine
  modified_base 22: mod_base = OTHER; note = thymine
  modified_base 24..27: mod_base = OTHER; note = thymine
  modified_base 29: mod_base = OTHER; note = thymine
  modified_base 33: mod_base = OTHER; note = thymine
  misc_feature 1..34: note = hSOD1 crRNA 5 Fwd primer
  source 1..34: mol_type = other RNA
aaaccatcctttggcccaccgtgttttctggata

SEQ ID NO: 13 - RNA, 34 nt, synthetic construct
  modified_base 5: mod_base = OTHER; note = thymine
  modified_base 7: mod_base = OTHER; note = thymine
  modified_base 21: mod_base = OTHER; note = thymine
  modified_base 33: mod_base = OTHER; note = thymine
  misc_feature 1..34: note = hSOD1 crRNA 5 Rev primer
  source 1..34: mol_type = other RNA
aaaatatccagaaaacacggtgggccaaaggatg

SEQ ID NO: 14 - RNA, 34 nt, synthetic construct
  modified_base 6: mod_base = OTHER; note = thymine
  modified_base 8: mod_base = OTHER; note = thymine
  modified_base 10..11: mod_base = OTHER; note = thymine
  modified_base 14: mod_base = OTHER; note = thymine
  modified_base 17..19: mod_base = OTHER; note = thymine
  modified_base 29: mod_base = OTHER; note = thymine
  modified_base 31..34: mod_base = OTHER; note = thymine
  misc_feature 1..34: note = hSOD1 crRNA 6 Fwd primer
  source 1..34: mol_type = other RNA
aaacctctcttcatcctttggcccaccgtgtttt

SEQ ID NO: 15 - RNA, 34 nt, synthetic construct
  modified_base 14: mod_base = OTHER; note = thymine
  modified_base 26: mod_base = OTHER; note = thymine
  misc_feature 1..34: note = hSOD1 crRNA 6 Rev primer
  source 1..34: mol_type = other RNA
aaaaaaaacacggtgggccaaaggatgaagagag

SEQ ID NO: 16 - RNA, 34 nt, synthetic construct
  modified_base 12: mod_base = OTHER; note = thymine
  modified_base 14: mod_base = OTHER; note = thymine
  modified_base 21: mod_base = OTHER; note = thymine
  modified_base 25: mod_base = OTHER; note = thymine
  modified_base 27: mod_base = OTHER; note = thymine
  modified_base 29..30: mod_base = OTHER; note = thymine
  modified_base 33: mod_base = OTHER; note = thymine
  misc_feature 1..34: note = hSOD1 crRNA 7 Fwd primer
  source 1..34: mol_type = other RNA
aaacgcccaagtctccaacatgcctctcttcatc

SEQ ID NO: 17 - RNA, 34 nt, synthetic construct
  modified_base 7: mod_base = OTHER; note = thymine
  modified_base 19: mod_base = OTHER; note = thymine
  modified_base 21..22: mod_base = OTHER; note = thymine
  modified_base 29..30: mod_base = OTHER; note = thymine
  misc_feature 1..34: note = hSOD1 crRNA 7 Rev primer
  source 1..34: mol_type = other RNA
aaaagatgaagagaggcatgttggagacttgggc

SEQ ID NO: 18 - RNA, 34 nt, synthetic construct
  modified_base 11: mod_base = OTHER; note = thymine
  modified_base 13: mod_base = OTHER; note = thymine
  modified_base 22: mod_base = OTHER; note = thymine
  modified_base 25: mod_base = OTHER; note = thymine
  modified_base 30: mod_base = OTHER; note = thymine
  modified_base 33: mod_base = OTHER; note = thymine
  misc_feature 1..34: note = hSOD1 crRNA 8 Fwd primer
  source 1..34: mol_type = other RNA
aaaccaccagtgtgcggccaatgatgcaatggtc

SEQ ID NO: 19 - RNA, 34 nt, synthetic construct
  modified_base 10..11: mod_base = OTHER; note = thymine
  modified_base 15: mod_base = OTHER; note = thymine
  modified_base 18..19: mod_base = OTHER; note = thymine
  modified_base 30: mod_base = OTHER; note = thymine
  modified_base 33: mod_base = OTHER; note = thymine
  misc_feature 1..34: note = hSOD1 crRNA 8 Rev primer
  source 1..34: mol_type = other RNA
aaaagaccattgcatcattggccgcacactggtg

SEQ ID NO: 20 - RNA, 34 nt, synthetic construct
  modified_base 5..7: mod_base = OTHER; note = thymine
  modified_base 15: mod_base = OTHER; note = thymine
  modified_base 18: mod_base = OTHER; note = thymine
  modified_base 20: mod_base = OTHER; note = thymine
  modified_base 23..27: mod_base = OTHER; note = thymine
  modified_base 30: mod_base = OTHER; note = thymine
  misc_feature 1..34: note = hSOD1 crRNA 9 Fwd primer
  source 1..34: mol_type = other RNA
aaactttgcccaagtcatctgctttttcatggac

SEQ ID NO: 21 - RNA, 34 nt, synthetic construct
  modified_base 6: mod_base = OTHER; note = thymine
  modified_base 10: mod_base = OTHER; note = thymine
  modified_base 22: mod_base = OTHER; note = thymine
  modified_base 26..27: mod_base = OTHER; note = thymine
  misc_feature 1..34: note = hSOD1 crRNA 9 Rev primer
  source 1..34: mol_type = other RNA
aaaagtccatgaaaaagcagatgacttgggcaaa

SEQ ID NO: 22 - RNA, 34 nt, synthetic construct
  modified_base 5: mod_base = OTHER; note = thymine
  modified_base 8: mod_base = OTHER; note = thymine
  modified_base 10: mod_base = OTHER; note = thymine
  modified_base 13..17: mod_base = OTHER; note = thymine
  modified_base 20: mod_base = OTHER; note = thymine
  modified_base 31: mod_base = OTHER; note = thymine
  modified_base 33: mod_base = OTHER; note = thymine
  misc_feature 1..34: note = hSOD1 crRNA 10 Fwd primer
  source 1..34: mol_type = other RNA
aaactcatctgctttttcatggaccaccagtgtg

SEQ ID NO: 23 - RNA, 34 nt, synthetic construct
  modified_base 10: mod_base = OTHER; note = thymine
  modified_base 13: mod_base = OTHER; note = thymine
  modified_base 16: mod_base = OTHER; note = thymine
  modified_base 20: mod_base = OTHER; note = thymine
  modified_base 32: mod_base = OTHER; note = thymine
  misc_feature 1..34: note = hSOD1 crRNA 10 Rev primer
  source 1..34: mol_type = other RNA
aaaacacactggtggtccatgaaaaagcagatga

SEQ ID NO: 24 - RNA, 34 nt, synthetic construct
  modified_base 7..8: mod_base = OTHER; note = thymine
  modified_base 11: mod_base = OTHER; note = thymine
  modified_base 16..19: mod_base = OTHER; note = thymine
  modified_base 26: mod_base = OTHER; note = thymine
  modified_base 32: mod_base = OTHER; note = thymine
  misc_feature 1..34: note = HTT crRNA 1 Fwd primer
  source 1..34: mol_type = other RNA
aaacccttcatcagcttttccagggtcgccatgg

SEQ ID NO: 25 - RNA, 34 nt, synthetic construct
  modified_base 8: mod_base = OTHER; note = thymine
  modified_base 17: mod_base = OTHER; note = thymine
  modified_base 26: mod_base = OTHER; note = thymine
  modified_base 29: mod_base = OTHER; note = thymine
  misc_feature 1..34: note = HTT crRNA 1 Rev primer
  source 1..34: mol_type = other RNA
aaaaccatggcgaccctggaaaagctgatgaagg

SEQ ID NO: 26 - RNA, 34 nt, synthetic construct
  modified_base 6: mod_base = OTHER; note = thymine
  modified_base 12: mod_base = OTHER; note = thymine
  modified_base 27: mod_base = OTHER; note = thymine
  modified_base 29: mod_base = OTHER; note = thymine
  modified_base 33: mod_base = OTHER; note = thymine
  misc_feature 1..34: note = HTT crRNA 2 Fwd primer
  source 1..34: mol_type = other RNA
aaacctgcggctgaggcagcagcggctgtgcctg

SEQ ID NO: 27 - RNA, 34 nt, synthetic construct
  modified_base 18: mod_base = OTHER; note = thymine
  modified_base 21: mod_base = OTHER; note = thymine
  modified_base 25: mod_base = OTHER; note = thymine
  misc_feature 1..34: note = HTT crRNA 2 Rev primer
  source 1..34: mol_type = other RNA
aaaacaggcacagccgctgctgcctcagccgcag

SEQ ID NO: 28 - RNA, 34 nt, synthetic construct
  modified_base 9: mod_base = OTHER; note = thymine
  modified_base 12: mod_base = OTHER; note = thymine
  modified_base 32: mod_base = OTHER; note = thymine
  misc_feature 1..34: note = HTT crRNA 3 Fwd primer
  source 1..34: mol_type = other RNA
aaaccggctcctcagccacagccgggccgggtgg

SEQ ID NO: 29 - RNA, 34 nt, synthetic construct
  modified_base 19: mod_base = OTHER; note = thymine
  modified_base 21: mod_base = OTHER; note = thymine
  modified_base 25: mod_base = OTHER; note = thymine
  misc_feature 1..34: note = HTT crRNA 3 Rev primer
  source 1..34: mol_type = other RNA
aaaaccacccggcccggctgtggctgaggagccg

SEQ ID NO: 30 - RNA, 34 nt, synthetic construct
  modified_base 6: mod_base = OTHER; note = thymine
  modified_base 10: mod_base = OTHER; note = thymine
  modified_base 19: mod_base = OTHER; note = thymine
  modified_base 22: mod_base = OTHER; note = thymine
  misc_feature 1..34: note = HTT crRNA 4 Fwd primer
  source 1..34: mol_type = other RNA
aaacgtcggtgcagcggctcctcagccacagccg

SEQ ID NO: 31 - RNA, 34 nt, synthetic construct
  modified_base 9: mod_base = OTHER; note = thymine
  modified_base 11: mod_base = OTHER; note = thymine
  modified_base 15: mod_base = OTHER; note = thymine
  modified_base 26: mod_base = OTHER; note = thymine
  misc_feature 1..34: note = HTT crRNA 4 Rev primer
  source 1..34: mol_type = other RNA
aaaacggctgtggctgaggagccgctgcaccgac

SEQ ID NO: 32 - RNA, 20 nt, synthetic construct
  modified_base 4: mod_base = OTHER; note = thymine
  modified_base 6: mod_base = OTHER; note = thymine
  modified_base 10: mod_base = OTHER; note = thymine
  modified_base 13: mod_base = OTHER; note = thymine
  misc_feature 1..20: note = qPCR-hSOD1-Fwd primer
  source 1..20: mol_type = other RNA
caatgtgactgctgacaaag

SEQ ID NO: 33 - RNA, 19 nt, synthetic construct
  modified_base 2: mod_base = OTHER; note = thymine
  modified_base 11: mod_base = OTHER; note = thymine
  modified_base 14: mod_base = OTHER; note = thymine
  modified_base 19: mod_base = OTHER; note = thymine
  misc_feature 1..19: note = qPCR-hSOD1-Rev primer
  source 1..19: mol_type = other RNA
gtgcggccaatgatgcaat

SEQ ID NO: 34 - RNA, 21 nt, synthetic construct
  modified_base 3: mod_base = OTHER; note = thymine
  modified_base 8: mod_base = OTHER; note = thymine
  modified_base 13: mod_base = OTHER; note = thymine
  modified_base 15: mod_base = OTHER; note = thymine
  misc_feature 1..21: note = qPCR-GAPDH-Fwd primer
  source 1..21: mol_type = other RNA
cctgacctgccgtctagaaaa

SEQ ID NO: 35 - RNA, 18 nt, synthetic construct
  modified_base 2: mod_base = OTHER; note = thymine
  modified_base 11: mod_base = OTHER; note = thymine
  modified_base 14..15: mod_base = OTHER; note = thymine
  misc_feature 1..18: note = qPCR-GAPDH-Rev primer
  source 1..18: mol_type = other RNA
ctccgacgcctgcttcac

SEQ ID NO: 36 - RNA, 24 nt, synthetic construct
  modified_base 6: mod_base = OTHER; note = thymine
  modified_base 20: mod_base = OTHER; note = thymine
  modified_base 23: mod_base = OTHER; note = thymine
  misc_feature 1..24: note = qPCR-msBeta-Actin-Fwd primer
  source 1..24: mol_type = other RNA
cacgatggaggggccggactcatc

SEQ ID NO: 37 - RNA, 24 nt, synthetic construct
  modified_base 1: mod_base = OTHER; note = thymine
  modified_base 9: mod_base = OTHER; note = thymine
  modified_base 11: mod_base = OTHER; note = thymine
  modified_base 13: mod_base = OTHER; note = thymine
  modified_base 24: mod_base = OTHER; note = thymine
  misc_feature 1..24: note = qPCR-msBeta-Actin-Rev primer
  source 1..24: mol_type = other RNA
taaagacctctatgccaacacagt

SEQ ID NO: 38 - RNA, 18 nt, synthetic construct
  modified_base 4: mod_base = OTHER; note = thymine
  modified_base 11: mod_base = OTHER; note = thymine
  misc_feature 1..18: note = qPCR-HTT-Fwd primer
  source 1..18: mol_type = other RNA
gggtccaagatggacggc

SEQ ID NO: 39 - RNA, 19 nt, synthetic construct
  modified_base 14: mod_base = OTHER; note = thymine
  modified_base 18: mod_base = OTHER; note = thymine
  misc_feature 1..19: note = qPCR-HTT-Rev primer
  source 1..19: mol_type = other RNA
agcaccggggcaatgaatg

SEQ ID NO: 40 - RNA, 20 nt, synthetic construct
  modified_base 4..5: mod_base = OTHER; note = thymine
  modified_base 7: mod_base = OTHER; note = thymine
  modified_base 10..11: mod_base = OTHER; note = thymine
  modified_base 20: mod_base = OTHER; note = thymine
  misc_feature 1..20: note = qPCR-bGHpA-Fwd primer
  source 1..20: mol_type = other RNA
gccttctagttgccagccat

SEQ ID NO: 41 - RNA, 19 nt, synthetic construct
  modified_base 7..8: mod_base = OTHER; note = thymine
  modified_base 15: mod_base = OTHER; note = thymine
  misc_feature 1..19: note = qPCR-bGHpA-Rev primer
  source 1..19: mol_type = other RNA
ggcaccttccagggtcaag

SEQ ID NO: 42 - RNA, 19 nt, synthetic construct
  modified_base 12: mod_base = OTHER; note = thymine
  modified_base 16: mod_base = OTHER; note = thymine
  modified_base 18: mod_base = OTHER; note = thymine
  misc_feature 1..19: note = qPCR-Zfp82-Fwd primer
  source 1..19: mol_type = other RNA
acggggacaactcactctc

SEQ ID NO: 43 - RNA, 19 nt, synthetic construct
  modified_base 12: mod_base = OTHER; note = thymine
  modified_base 16: mod_base = OTHER; note = thymine
  modified_base 18: mod_base = OTHER; note = thymine
  misc_feature 1..19: note = qPCR-Zfp82-Rev primer
  source 1..19: mol_type = other RNA
acggggacaactcactctc

SEQ ID NO: 44 - RNA, 20 nt, synthetic construct
  modified_base 1: mod_base = OTHER; note = thymine
  modified_base 8: mod_base = OTHER; note = thymine
  modified_base 13: mod_base = OTHER; note = thymine
  misc_feature 1..20: note = qPCR-Npcd-Fwd primer
  source 1..20: mol_type = other RNA
tgggcgatcaagtacagcac

SEQ ID NO: 45 - RNA, 21 nt, synthetic construct
  modified_base 1..3: mod_base = OTHER; note = thymine
  modified_base 7..9: mod_base = OTHER; note = thymine
  modified_base 13: mod_base = OTHER; note = thymine
  modified_base 18: mod_base = OTHER; note = thymine
  modified_base 20..21: mod_base = OTHER; note = thymine
  misc_feature 1..21: note = qPCR-Npcd-Rev primer
  source 1..21: mol_type = other RNA
tttgggtttaggtcccctctt

SEQ ID NO: 46 - RNA, 21 nt, synthetic construct
  modified_base 1: mod_base = OTHER; note = thymine
  modified_base 8..10: mod_base = OTHER; note = thymine
  modified_base 15: mod_base = OTHER; note = thymine
  modified_base 17: mod_base = OTHER; note = thymine
  misc_feature 1..21: note = qPCR-Cbx6-Fwd primer
  source 1..21: mol_type = other RNA
tggcacatttgcgctctacaa

SEQ ID NO: 47 - RNA, 19 nt, synthetic construct
  modified_base 2: mod_base = OTHER; note = thymine
  modified_base 10: mod_base = OTHER; note = thymine
  modified_base 14..15: mod_base = OTHER; note = thymine
  modified_base 19: mod_base = OTHER; note = thymine
  misc_feature 1..19: note = qPCR-Cbx6-Rev primer
  source 1..19: mol_type = other RNA
ctccgggagtagcttgggt

SEQ ID NO: 48 - RNA, 22 nt, synthetic construct
  modified_base 2: mod_base = OTHER; note = thymine
  modified_base 6..7: mod_base = OTHER; note = thymine
  modified_base 14: mod_base = OTHER; note = thymine
  modified_base 16..17: mod_base = OTHER; note = thymine
  modified_base 20: mod_base = OTHER; note = thymine
  misc_feature 1..22: note = qPCR-Trim6-Fwd primer
  source 1..22: mol_type = other RNA
atgacttcaacagtcttggtgg

SEQ ID NO: 49 - RNA, 21 nt, synthetic construct
  modified_base 1..2: mod_base = OTHER; note = thymine
  modified_base 10: mod_base = OTHER; note = thymine
  modified_base 13: mod_base = OTHER; note = thymine
  modified_base 20: mod_base = OTHER; note = thymine
  misc_feature 1..21: note = qPCR-Trim6-Rev primer
  source 1..21: mol_type = other RNA
ttcccaggctgataggaggtc

SEQ ID NO: 50 - RNA, 21 nt, synthetic construct
  modified_base 2: mod_base = OTHER; note = thymine
  modified_base 17..18: mod_base = OTHER; note = thymine
  misc_feature 1..21: note = qPCR-Pdcd5-Fwd primer
  source 1..21: mol_type = other RNA
atggcggacgaagaacttgag

SEQ ID NO: 51 - RNA, 19 nt, synthetic construct
  modified_base 6: mod_base = OTHER; note = thymine
  modified_base 10: mod_base = OTHER; note = thymine
  modified_base 13: mod_base = OTHER; note = thymine
  misc_feature 1..19: note = qPCR-Pdcd5-Rev primer
  source 1..19: mol_type = other RNA
cgggctgactgatccagaa

SEQ ID NO: 52 - RNA, 22 nt, synthetic construct
  modified_base 3: mod_base = OTHER; note = thymine
  modified_base 5: mod_base = OTHER; note = thymine
  modified_base 11: mod_base = OTHER; note = thymine
  modified_base 14: mod_base = OTHER; note = thymine
  modified_base 20: mod_base = OTHER; note = thymine
  misc_feature 1..22: note = qPCR-Cpsf6-Fwd primer
  source 1..22: mol_type = other RNA
catctgcaaataatggcgatgc

SEQ ID NO: 53 - RNA, 23 nt, synthetic construct
  modified_base 2: mod_base = OTHER; note = thymine
  modified_base 5: mod_base = OTHER; note = thymine
  modified_base 7: mod_base = OTHER; note = thymine
  modified_base 9..10: mod_base = OTHER; note = thymine
  modified_base 12: mod_base = OTHER; note = thymine
  modified_base 19: mod_base = OTHER; note = thymine
  modified_base 21..22: mod_base = OTHER; note = thymine
  misc_feature 1..23: note = qPCR-Cpsf6-Rev primer
  source 1..23: mol_type = other RNA
ctcatctgttgtccaccatgtta

SEQ ID NO: 54 - RNA, 21 nt, synthetic construct
  modified_base 2: mod_base = OTHER; note = thymine
  modified_base 9..10: mod_base = OTHER; note = thymine
  misc_feature 1..21: note = qPCR-Prpf4b-Fwd primer
  source 1..21: mol_type = other RNA
atcgaggcttccgacaaagaa

SEQ ID NO: 55 - RNA, 22 nt, synthetic construct
  modified_base 1: mod_base = OTHER; note = thymine
  modified_base 5..8: mod_base = OTHER; note = thymine
  modified_base 11: mod_base = OTHER; note = thymine
  modified_base 15: mod_base = OTHER; note = thymine
  modified_base 17: mod_base = OTHER; note = thymine
  modified_base 19: mod_base = OTHER; note = thymine
  modified_base 22: mod_base = OTHER; note = thymine
  misc_feature 1..22: note = qPCR-Prpf4b-Rev primer
  source 1..22: mol_type = other RNA
tgccttttcgtgaatctctcct

SEQ ID NO: 56 - RNA, 19 nt, synthetic construct
  modified_base 2: mod_base = OTHER; note = thymine
  modified_base 8: mod_base = OTHER; note = thymine
  modified_base 17: mod_base = OTHER; note = thymine
  modified_base 19: mod_base = OTHER; note = thymine
  misc_feature 1..19: note = qPCR-msSOD1-Fwd primer
  source 1..19: mol_type = other RNA
atggcgatgaaagcggtgt

SEQ ID NO: 57 - RNA, 23 nt, synthetic construct
  modified_base 3..4: mod_base = OTHER; note = thymine
  modified_base 6: mod_base = OTHER; note = thymine
  modified_base 8: mod_base = OTHER; note = thymine
  modified_base 10..11: mod_base = OTHER; note = thymine
  modified_base 13: mod_base = OTHER; note = thymine
  modified_base 19: mod_base = OTHER; note = thymine
  modified_base 22: mod_base = OTHER; note = thymine
  misc_feature 1..23: note = qPCR-msSOD1-Rev primer
  source 1..23: mol_type = other RNA
ccttgtgtattgtccccatactg

SEQ ID NO: 58 - RNA, 30 nt, synthetic construct
  modified_base 2: mod_base = OTHER; note = thymine
  modified_base 11: mod_base = OTHER; note = thymine
  modified_base 14: mod_base = OTHER; note = thymine
  modified_base 18..20: mod_base = OTHER; note = thymine
  misc_feature 1..30: note = Human SOD1 mRNA targeted sequence
  source 1..30: mol_type = other RNA
gtgcagggcatcatcaatttcgcgcagaag

SEQ ID NO: 59 - RNA, 30 nt, synthetic construct
  modified_base 6: mod_base = OTHER; note = thymine
  modified_base 9: mod_base = OTHER; note = thymine
  modified_base 17: mod_base = OTHER; note = thymine
  modified_base 23: mod_base = OTHER; note = thymine
  modified_base 25: mod_base = OTHER; note = thymine
  misc_feature 1..30: note = Human SOD1 mRNA targeted sequence
  source 1..30: mol_type = other RNA
gaaagtaatggaccagtgaaggtgtgggga

SEQ ID NO: 60 - RNA, 30 nt, synthetic construct
  modified_base 1: mod_base = OTHER; note = thymine
  modified_base 17: mod_base = OTHER; note = thymine
  modified_base 20: mod_base = OTHER; note = thymine
  modified_base 28: mod_base = OTHER; note = thymine
  misc_feature 1..30: note = Human SOD1 mRNA targeted sequence
  source 1..30: mol_type = other RNA
tcgagcagaaggaaagtaatggaccagtga

SEQ ID NO: 61 - RNA, 30 nt, synthetic construct
  modified_base 2: mod_base = OTHER; note = thymine
  modified_base 4..5: mod_base = OTHER; note = thymine
  modified_base 8: mod_base = OTHER; note = thymine
  modified_base 12..14: mod_base = OTHER; note = thymine
  modified_base 20: mod_base = OTHER; note = thymine
  modified_base 23: mod_base = OTHER; note = thymine
  misc_feature 1..30: note = Human SOD1 mRNA targeted sequence
  source 1..30: mol_type = other RNA
atgttcatgagtttggagataatacagcag

SEQ ID NO: 62 - RNA, 30 nt, synthetic construct
  modified_base 3: mod_base = OTHER; note = thymine
  modified_base 5: mod_base = OTHER; note = thymine
  modified_base 11: mod_base = OTHER; note = thymine
  modified_base 17: mod_base = OTHER; note = thymine
  modified_base 20: mod_base = OTHER; note = thymine
  modified_base 24..26: mod_base = OTHER; note = thymine
  modified_base 29: mod_base = OTHER; note = thymine
  misc_feature 1..30: note = Human SOD1 mRNA targeted sequence
  source 1..30: mol_type = other RNA
gctgtaccagtgcaggtcctcactttaatc

SEQ ID NO: 63 - RNA, 30 nt, synthetic construct
  modified_base 10: mod_base = OTHER; note = thymine
  modified_base 22: mod_base = OTHER; note = thymine
  misc_feature 1..30: note = Human SOD1 mRNA targeted sequence
  source 1..30: mol_type = other RNA
aaaacacggtgggccaaaggatgaagagag

SEQ ID NO: 64 - RNA, 30 nt, synthetic construct
  modified_base 3: mod_base = OTHER; note = thymine
  modified_base 15: mod_base = OTHER; note = thymine
  modified_base 17..18: mod_base = OTHER; note = thymine
  modified_base 25..26: mod_base = OTHER; note = thymine
  misc_feature 1..30: note = Human SOD1 mRNA targeted sequence
  source 1..30: mol_type = other RNA
gatgaagagaggcatgttggagacttgggc

SEQ ID NO: 65 - RNA, 30 nt, synthetic construct
  modified_base 6..7: mod_base = OTHER; note = thymine
  modified_base 11: mod_base = OTHER; note = thymine
  modified_base 14..15: mod_base = OTHER; note = thymine
  modified_base 26: mod_base = OTHER; note = thymine
  modified_base 29: mod_base = OTHER; note = thymine
  misc_feature 1..30: note = Human SOD1 mRNA targeted sequence
  source 1..30: mol_type = other RNA
gaccattgcatcattggccgcacactggtg

SEQ ID NO: 66 - RNA, 30 nt, synthetic construct
  modified_base 2: mod_base = OTHER; note = thymine
  modified_base 6: mod_base = OTHER; note = thymine
  modified_base 18: mod_base = OTHER; note = thymine
  modified_base 22..23: mod_base = OTHER; note = thymine
  misc_feature 1..30: note = Human SOD1 mRNA targeted sequence
  source 1..30: mol_type = other RNA
gtccatgaaaaacgagatcacttgggcaaa

SEQ ID NO: 67 - RNA, 30 nt, synthetic construct
  modified_base 6: mod_base = OTHER; note = thymine
  modified_base 9: mod_base = OTHER; note = thymine
  modified_base 12: mod_base = OTHER; note = thymine
  modified_base 16: mod_base = OTHER; note = thymine
  modified_base 28: mod_base = OTHER; note = thymine
  misc_feature 1..30: note = Human SOD1 mRNA targeted sequence
  source 1..30: mol_type = other RNA
cacactggtggtccatgaaaaagcagatga

SEQ ID NO: 68 - RNA, 30 nt, synthetic construct
  modified_base 4: mod_base = OTHER; note = thymine
  modified_base 13: mod_base = OTHER; note = thymine
  modified_base 22: mod_base = OTHER; note = thymine
  modified_base 25: mod_base = OTHER; note = thymine
  misc_feature 1..30: note = Human HTT exon 1 targeted sequence
  source 1..30: mol_type = other RNA
ccatggcgaccctggaaaagctgatgaagg

SEQ ID NO: 69 - RNA, 30 nt, synthetic construct
  modified_base 14: mod_base = OTHER; note = thymine
  modified_base 17: mod_base = OTHER; note = thymine
  modified_base 21: mod_base = OTHER; note = thymine
  misc_feature 1..30: note = Human HTT exon 1 targeted sequence
  source 1..30: mol_type = other RNA
caggcacagccgctgctgcctcagccgcag

SEQ ID NO: 70 - RNA, 30 nt, synthetic construct
  modified_base 15: mod_base = OTHER; note = thymine
  modified_base 17: mod_base = OTHER; note = thymine
  modified_base 21: mod_base = OTHER; note = thymine
  misc_feature 1..30: note = Human HTT exon 1 targeted sequence
  source 1..30: mol_type = other RNA
ccacccggcccggctgtggctgaggagccg

SEQ ID NO: 71 - RNA, 30 nt, synthetic construct
  modified_base 5: mod_base = OTHER; note = thymine
  modified_base 7: mod_base = OTHER; note = thymine
  modified_base 11: mod_base = OTHER; note = thymine
  modified_base 22: mod_base = OTHER; note = thymine
  misc_feature 1..30: note = Human HTT exon 1 targeted sequence
  source 1..30: mol_type = other RNA
cggctgtggctgaggagccgctgcaccgac

SEQ ID NO: 72 - RNA, 30 nt, synthetic construct; identical to SEQ ID NO: 60
  modified_base 1: mod_base = OTHER; note = thymine
  modified_base 17: mod_base = OTHER; note = thymine
  modified_base 20: mod_base = OTHER; note = thymine
  modified_base 28: mod_base = OTHER; note = thymine
  misc_feature 1..30: note = hSOD1 targeted sequence
  source 1..30: mol_type = other RNA

SEQ ID NO: 73 - RNA, 30 nt, synthetic construct
  modified_base 8: mod_base = OTHER; note = thymine
  modified_base 17: mod_base = OTHER; note = thymine
  modified_base 20: mod_base = OTHER; note = thymine
  modified_base 27: mod_base = OTHER; note = thymine
  misc_feature 1..30: note = Zfp82 targeted sequence
  source 1..30: mol_type = other RNA
caaagactaaggaaagtaatggacactgaa

SEQ ID NO: 74 - RNA, 30 nt, synthetic construct
  modified_base 1: mod_base = OTHER; note = thymine
  modified_base 23: mod_base = OTHER; note = thymine
  modified_base 28: mod_base = OTHER; note = thymine
  modified_base 30: mod_base = OTHER; note = thymine
  misc_feature 1..30: note = Npcd targeted sequence
  source 1..30: mol_type = other RNA
tcgagcagaaggaaagggaacgtgagctgt

SEQ ID NO: 75 - RNA, 30 nt, synthetic construct; identical to SEQ ID NO: 74
  modified_base 1: mod_base = OTHER; note = thymine
  modified_base 23: mod_base = OTHER; note = thymine
  modified_base 28: mod_base = OTHER; note = thymine
  modified_base 30: mod_base = OTHER; note = thymine
  misc_feature 1..30: note = Cbx6 targeted sequence
  source 1..30: mol_type = other RNA

SEQ ID NO: 76 - RNA, 30 nt, synthetic construct
  modified_base 17: mod_base = OTHER; note = thymine
  modified_base 20: mod_base = OTHER; note = thymine
  modified_base 23..24: mod_base = OTHER; note = thymine
  misc_feature 1..30: note = Trim6 targeted sequence
  source 1..30: mol_type = other RNA
aagggcagaaggaaagtaatacttcgagaa

SEQ ID NO: 77 - RNA, 30 nt, synthetic construct
  modified_base 1..2: mod_base = OTHER; note = thymine
  modified_base 17: mod_base = OTHER; note = thymine
  modified_base 20: mod_base = OTHER; note = thymine
  modified_base 25: mod_base = OTHER; note = thymine
  modified_base 30: mod_base = OTHER; note = thymine
  misc_feature 1..30: note = Pdcd5 targeted sequence
  source 1..30: mol_type = other RNA
ttcaacagaagaaaagtaatggactccgat

SEQ ID NO: 78 - RNA, 30 nt, synthetic construct
  modified_base 2: mod_base = OTHER; note = thymine
  modified_base 11..12: mod_base = OTHER; note = thymine
  modified_base 17: mod_base = OTHER; note = thymine
  modified_base 20: mod_base = OTHER; note = thymine
  misc_feature 1..30: note = Cpsf6 targeted sequence
  source 1..30: mol_type = other RNA
ataacaacacttaaagtaatggaccagcag

SEQ ID NO: 79 - RNA, 30 nt, synthetic construct
  modified_base 17: mod_base = OTHER; note = thymine
  modified_base 27: mod_base = OTHER; note = thymine
  misc_feature 1..30: note = Prp4fb targeted sequence
  source 1..30: mol_type = other RNA
aggagcagaagcaaagtaaaggaagataaa

SEQ ID NO: 80 - protein, 7 aa, synthetic construct
  REGION 1..7: note = NLS SV40 large T antigen
PKKKRKV

SEQ ID NO: 81 - protein, 16 aa, synthetic construct
  REGION 1..16: note = NLS nucleoplasmin
KRPAATKKAGQAKKKK

SEQ ID NO: 82 - protein, 7 aa, synthetic construct
  REGION 1..7: note = NLS p54
RIRKKLR

SEQ ID NO: 83 - protein, 5 aa, synthetic construct
  REGION 1..5: note = NLS SOX9
PRRRK

SEQ ID NO: 84 - protein, 9 aa, synthetic construct
  REGION 1..9: note = NLS NS5A
PPRKKRTVV

SEQ ID NO: 85 - protein, 11 aa, synthetic construct
  REGION 1..11: note = NLS
SPKKKRKVEAS

SEQ ID NO: 86 - protein, 11 aa, synthetic construct
  REGION 1..11: note = SV40 large T antigen NLS
GPKKKRKVAAA

SEQ ID NO: 87 - RNA, 27 nt, synthetic construct
  modified_base 1: mod_base = OTHER; note = thymine
  modified_base 7: mod_base = OTHER; note = thymine
  modified_base 12: mod_base = OTHER; note = thymine
  modified_base 14..15: mod_base = OTHER; note = thymine
  modified_base 21..22: mod_base = OTHER; note = thymine
  modified_base 27: mod_base = OTHER; note = thymine
  misc_feature 1..27: note = hemagglutinin epitope tag
  source 1..27: mol_type = other RNA
tacccatacgatgttccagattacgct

SEQ ID NO: 88 - RNA, 27 nt, synthetic construct
  modified_base 1: mod_base = OTHER; note = thymine
  modified_base 3: mod_base = OTHER; note = thymine
  modified_base 7: mod_base = OTHER; note = thymine
  modified_base 9: mod_base = OTHER; note = thymine
  modified_base 12: mod_base = OTHER; note = thymine
  modified_base 14..15: mod_base = OTHER; note = thymine
  modified_base 21..22: mod_base = OTHER; note = thymine
  modified_base 24: mod_base = OTHER; note = thymine
  modified_base 27: mod_base = OTHER; note = thymine
  misc_feature 1..27: note = hemagglutinin epitope tag
  source 1..27: mol_type = other RNA
tatccatatgatgttccagattatgct

SEQ ID NO: 89 - protein, 9 aa, synthetic construct
  REGION 1..9: note = hemagglutinin epitope tag
YPYDVPDYA

SEQ ID NO: 90 - DNA, 35 bp, synthetic construct
  misc_feature 1..35: note = Cas13-specific direct repeat
  source 1..35: mol_type = other DNA
gaaacaccgaacccctaccaactggtcggggtttg

SEQ ID NO: 91 - DNA, 241 bp, synthetic construct
  misc_feature 1..241: note = U6 promoter
  source 1..241: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 92 - RNA, 7043 nt, synthetic construct
  misc_feature 1..7043: note = Nucleotide sequence of the pAAV-CAG-RfxCas13d-U6-crRNA vector
  source 1..7043: mol_type = other RNA
  modified_base 2: mod_base = OTHER; note = thymine
  modified_base 7..10: mod_base = OTHER; note = thymine
  modified_base 13: mod_base = OTHER; note = thymine
  modified_base 18..21: mod_base = OTHER; note = thymine
  modified_base 24: mod_base = OTHER; note = thymine
  modified_base 29: mod_base = OTHER; note = thymine
  modified_base 31: mod_base = OTHER; note = thymine
  modified_base 34: mod_base = OTHER; note = thymine
  modified_base 44: mod_base = OTHER; note = thymine
  modified_base 51: mod_base = OTHER; note = thymine
  modified_base 55: mod_base = OTHER; note = thymine
  modified_base 59: mod_base = OTHER; note = thymine
  modified_base 63: mod_base = OTHER; note = thymine
  modified_base 79: mod_base = OTHER; note = thymine
  modified_base 89..91: mod_base = OTHER; note = thymine
  modified_base 94: mod_base = OTHER; note = thymine
  modified_base 104: mod_base = OTHER; note = thymine
  modified_base 108: mod_base = OTHER; note = thymine
  modified_base 135: mod_base = OTHER; note = thymine
  modified_base 143: mod_base = OTHER; note = thymine
  modified_base 147: mod_base = OTHER; note = thymine
  modified_base 151: mod_base = OTHER; note = thymine
  modified_base 157..158: mod_base = OTHER; note = thymine
  modified_base 161: mod_base = OTHER; note = thymine
  modified_base 168: mod_base = OTHER; note = thymine
  modified_base 170: mod_base = OTHER; note = thymine
  modified_base 177..178: mod_base = OTHER; note = thymine
  modified_base 182: mod_base = OTHER; note = thymine
  modified_base 186..187: mod_base = OTHER; note = thymine
  modified_base 192: mod_base = OTHER; note = thymine
  modified_base 196: mod_base = OTHER; note = thymine
  modified_base 205: mod_base = OTHER; note = thymine
  modified_base 209: mod_base = OTHER; note = thymine
  modified_base 233..234: mod_base = OTHER; note = thymine
  modified_base 239: mod_base = OTHER; note = thymine
  modified_base 243: mod_base = OTHER; note = thymine
  modified_base 246: mod_base = OTHER; note = thymine
  modified_base 251: mod_base = OTHER; note = thymine
  modified_base 253: mod_base = OTHER; note = thymine
  modified_base 255..256: mod_base = OTHER; note = thymine
  modified_base 261: mod_base = OTHER; note = thymine
  modified_base 264: mod_base = OTHER; note = thymine
  modified_base 273: mod_base = OTHER; note = thymine
  modified_base 280..282: mod_base = OTHER; note = thymine
  modified_base 286..287: mod_base = OTHER; note = thymine
  modified_base 292: mod_base = OTHER; note = thymine
  modified_base 296: mod_base = OTHER; note = thymine
  modified_base 300: mod_base = OTHER; note = thymine
  modified_base 305: mod_base = OTHER; note = thymine
  modified_base 307..309: mod_base = OTHER; note = thymine
  modified_base 314: mod_base = OTHER; note = thymine
  modified_base 319: mod_base = OTHER; note = thymine
  modified_base 326..327: mod_base = OTHER; note = thymine
  modified_base 333: mod_base = OTHER; note = thymine
  modified_base 337: mod_base = OTHER; note = thymine
  modified_base 342: mod_base = OTHER; note = thymine
  modified_base 344: mod_base = OTHER; note = thymine
  modified_base 346: mod_base = OTHER; note = thymine
  modified_base 349: mod_base = OTHER; note = thymine
  modified_base 351: mod_base = OTHER; note = thymine
  modified_base 358: mod_base = OTHER; note = thymine
  modified_base 367: mod_base = OTHER; note = thymine
  modified_base 369..370: mod_base = OTHER; note = thymine
  modified_base 375: mod_base = OTHER; note = thymine
  modified_base 379: mod_base = OTHER; note = thymine
  modified_base 385: mod_base = OTHER; note = thymine
  modified_base 389: mod_base = OTHER; note = thymine
  modified_base 398: mod_base = OTHER; note = thymine
  modified_base 403..404: mod_base = OTHER; note = thymine
  modified_base 406: mod_base = OTHER; note = thymine
  modified_base 413: mod_base = OTHER; note = thymine
  modified_base 417: mod_base = OTHER; note = thymine
  modified_base 422..423: mod_base = OTHER; note = thymine
  modified_base 425: mod_base = OTHER; note = thymine
  modified_base 431..433: mod_base = OTHER; note = thymine
  modified_base 436: mod_base = OTHER; note = thymine
  modified_base 439..440: mod_base = OTHER; note = thymine
  modified_base 446: mod_base = OTHER; note = thymine
  modified_base 450: mod_base = OTHER; note = thymine
  modified_base 452: mod_base = OTHER; note = thymine
  modified_base 456: mod_base = OTHER; note = thymine
  modified_base 458..459: mod_base = OTHER; note = thymine
  modified_base 462: mod_base = OTHER; note = thymine
  modified_base 465: mod_base = OTHER; note = thymine
  modified_base 469: mod_base = OTHER; note = thymine
  modified_base 471..472: mod_base = OTHER; note = thymine
  modified_base 477: mod_base = OTHER; note = thymine
  modified_base 480: mod_base = OTHER; note = thymine
  modified_base 486: mod_base = OTHER; note = thymine
  modified_base 497..498: mod_base = OTHER; note = thymine
  modified_base 500: mod_base = OTHER; note = thymine
  modified_base 503..504: mod_base = OTHER; note = thymine
  modified_base 508: mod_base = OTHER; note = thymine
  modified_base 510: mod_base = OTHER; note = thymine
  modified_base 516: mod_base = OTHER; note = thymine
  modified_base 518: mod_base = OTHER; note = thymine
  modified_base 527: mod_base = OTHER; note = thymine
  modified_base 540..543: mod_base = OTHER; note = thymine
  modified_base 545: mod_base = OTHER; note = thymine
  modified_base 547..549: mod_base = OTHER; note = thymine
  modified_base 551..553: mod_base = OTHER; note = thymine
  modified_base 555..560: mod_base = OTHER; note = thymine
  modified_base 563..564: mod_base = OTHER; note = thymine
  modified_base 566..569: mod_base = OTHER; note = thymine
  modified_base 571: mod_base = OTHER; note = thymine
  modified_base 579: mod_base = OTHER; note = thymine
  modified_base 658: mod_base = OTHER; note = thymine
  modified_base 673: mod_base = OTHER; note = thymine
  modified_base 687: mod_base = OTHER; note = thymine
  modified_base 695..697: mod_base = OTHER; note = thymine
  modified_base 700..703: mod_base = OTHER; note = thymine
  modified_base 705: mod_base = OTHER; note = thymine
  modified_base 731: mod_base = OTHER; note = thymine
  modified_base 733: mod_base = OTHER; note = thymine
  modified_base 763: mod_base = OTHER; note = thymine
  modified_base 771: mod_base = OTHER; note = thymine
  modified_base 795: mod_base = OTHER; note = thymine
  modified_base 807: mod_base = OTHER; note = thymine
  modified_base 821: mod_base = OTHER; note = thymine
  modified_base 824..825: mod_base = OTHER; note = thymine
  modified_base 837: mod_base = OTHER; note = thymine
  modified_base 843: mod_base = OTHER; note = thymine
  modified_base 848: mod_base = OTHER; note = thymine
  modified_base 855: mod_base = OTHER; note = thymine
  modified_base 858: mod_base = OTHER; note = thymine
  modified_base 860: mod_base = OTHER; note = thymine
  modified_base 866: mod_base = OTHER; note = thymine
  modified_base 873: mod_base = OTHER; note = thymine
  modified_base 875: mod_base = OTHER; note = thymine
  modified_base 879: mod_base = OTHER; note = thymine
  modified_base 887..888: mod_base = OTHER; note = thymine
  modified_base 912: mod_base = OTHER; note = thymine
  modified_base 921: mod_base = OTHER; note = thymine
  modified_base 924: mod_base = OTHER; note = thymine
  modified_base 939: mod_base = OTHER; note = thymine
  modified_base 948: mod_base = OTHER; note = thymine
  modified_base 952: mod_base = OTHER; note = thymine
  modified_base 965..966: mod_base = OTHER; note = thymine
  modified_base 973: mod_base = OTHER; note = thymine
  modified_base 978: mod_base = OTHER; note = thymine
  modified_base 985: mod_base = OTHER; note = thymine
  modified_base 998: mod_base = OTHER; note = thymine
  modified_base 1000: mod_base = OTHER; note = thymine
  modified_base 1005: mod_base = OTHER; note = thymine
  modified_base 1019..1020: mod_base = OTHER; note = thymine
  modified_base 1027: mod_base = OTHER; note = thymine
  modified_base 1030: mod_base = OTHER; note = thymine
  modified_base 1037: mod_base = OTHER; note = thymine
  modified_base 1044: mod_base = OTHER; note = thymine
  modified_base 1047: mod_base = OTHER; note = thymine
  modified_base 1051: mod_base = OTHER; note = thymine
  modified_base 1060: mod_base = OTHER; note = thymine
  modified_base 1062: mod_base = OTHER; note = thymine
  modified_base 1064: mod_base = OTHER; note = thymine
  modified_base 1066: mod_base = OTHER; note = thymine
  modified_base 1077: mod_base = OTHER; note = thymine
  modified_base 1087: mod_base = OTHER; note = thymine
  modified_base 1089: mod_base = OTHER; note = thymine
  modified_base 1092: mod_base = OTHER; note = thymine
  modified_base 1098: mod_base = OTHER; note = thymine
  modified_base 1108: mod_base = OTHER; note = thymine
  modified_base 1110: mod_base = OTHER; note = thymine
  modified_base 1121: mod_base = OTHER; note = thymine
  modified_base 1124..1125: mod_base = OTHER; note = thymine
  modified_base 1138: mod_base = OTHER; note = thymine
  modified_base 1141: mod_base = OTHER; note = thymine
  modified_base 1147: mod_base = OTHER; note = thymine
  modified_base 1153: mod_base = OTHER; note = thymine
  modified_base 1155..1157: mod_base = OTHER; note = thymine
  modified_base 1159: mod_base = OTHER; note = thymine
  modified_base 1161: mod_base = OTHER; note = thymine
  modified_base 1167: mod_base = OTHER; note = thymine
  modified_base 1170: mod_base = OTHER; note = thymine
  modified_base 1174: mod_base = OTHER; note = thymine
  modified_base 1179: mod_base = OTHER; note = thymine
  modified_base 1182: mod_base = OTHER; note = thymine
  modified_base 1188..1189: mod_base = OTHER; note = thymine
  modified_base 1197: mod_base = OTHER; note = thymine
  modified_base 1200: mod_base = OTHER; note = thymine
  modified_base 1208: mod_base = OTHER; note = thymine
  modified_base 1212..1213: mod_base = OTHER; note = thymine
  modified_base 1226: mod_base = OTHER; note = thymine
  modified_base 1233: mod_base = OTHER; note = thymine
  modified_base 1240: mod_base = OTHER; note = thymine
  modified_base 1242: mod_base = OTHER; note = thymine
  modified_base 1244: mod_base = OTHER; note = thymine
  modified_base 1251: mod_base = OTHER; note = thymine
  modified_base 1255: mod_base = OTHER; note = thymine
  modified_base 1263..1264: mod_base = OTHER; note = thymine
  modified_base 1266..1267: mod_base = OTHER; note = thymine
  modified_base 1271..1272: mod_base = OTHER; note = thymine
  modified_base 1280..1281: mod_base = OTHER; note = thymine
  modified_base 1283: mod_base = OTHER; note = thymine
  modified_base 1290: mod_base = OTHER; note = thymine
  modified_base 1292: mod_base = OTHER; note = thymine
  modified_base 1294: mod_base = OTHER; note = thymine
  modified_base 1298: mod_base = OTHER; note = thymine
  modified_base 1307..1308: mod_base = OTHER; note = thymine
  modified_base 1327: mod_base = OTHER; note = thymine
  modified_base 1336..1338: mod_base = OTHER; note = thymine
  modified_base 1345: mod_base = OTHER; note = thymine
  modified_base 1351: mod_base = OTHER; note = thymine
  modified_base 1356: mod_base = OTHER; note = thymine
  modified_base 1359: mod_base = OTHER; note = thymine
  modified_base 1368: mod_base = OTHER; note = thymine
  modified_base 1379: mod_base = OTHER; note = thymine
  modified_base 1381: mod_base = OTHER; note = thymine
  modified_base 1388..1389: mod_base = OTHER; note = thymine
  modified_base 1397..1398: mod_base = OTHER; note = thymine
  modified_base 1401: mod_base = OTHER; note = thymine
  modified_base 1405: mod_base = OTHER; note = thymine
  modified_base 1416: mod_base = OTHER; note = thymine
  modified_base 1421: mod_base = OTHER; note = thymine
  modified_base 1423..1425: mod_base = OTHER; note = thymine
  modified_base 1436..1440: mod_base = OTHER; note = thymine
  modified_base 1459..1460: mod_base = OTHER; note = thymine
  modified_base 1464: mod_base = OTHER; note = thymine
  modified_base 1467: mod_base = OTHER; note = thymine
  modified_base 1471..1472: mod_base = OTHER; note = thymine
  modified_base 1484: mod_base = OTHER; note = thymine
  modified_base 1487: mod_base = OTHER; note = thymine
  modified_base 1489: mod_base = OTHER; note = thymine
  modified_base 1494..1495: mod_base = OTHER; note = thymine
  modified_base 1497: mod_base = OTHER; note = thymine
  modified_base 1503: mod_base = OTHER; note = thymine
  modified_base 1506: mod_base = OTHER; note = thymine
  modified_base 1515: mod_base = OTHER; note = thymine
  modified_base 1523: mod_base = OTHER; note = thymine
  modified_base 1527: mod_base = OTHER; note = thymine
  modified_base 1530: mod_base = OTHER; note = thymine
  modified_base 1534: mod_base = OTHER; note = thymine
  modified_base 1550: mod_base = OTHER; note = thymine
  modified_base 1557: mod_base = OTHER; note = thymine
  modified_base 1559: mod_base = OTHER; note = thymine
  modified_base 1568: mod_base = OTHER; note = thymine
  modified_base 1572: mod_base = OTHER; note = thymine
  modified_base 1574: mod_base = OTHER; note = thymine
  modified_base 1581: mod_base = OTHER; note = thymine
  modified_base 1585: mod_base = OTHER; note = thymine
  modified_base 1593: mod_base = OTHER; note = thymine
  modified_base 1604: mod_base = OTHER; note = thymine
  modified_base 1608: mod_base = OTHER; note = thymine
  modified_base 1610: mod_base = OTHER; note = thymine
  modified_base 1617: mod_base = OTHER; note = thymine
  modified_base 1622: mod_base = OTHER; note = thymine
  modified_base 1626: mod_base = OTHER; note = thymine
  modified_base 1628: mod_base = OTHER; note = thymine
  modified_base 1638: mod_base = OTHER; note = thymine
  modified_base 1645: mod_base = OTHER; note = thymine
  modified_base 1650: mod_base = OTHER; note = thymine
  modified_base 1658: mod_base = OTHER; note = thymine
  modified_base 1661..1662: mod_base = OTHER; note = thymine
  modified_base 1664: mod_base = OTHER; note = thymine
  modified_base 1673: mod_base = OTHER; note = thymine
  modified_base 1686: mod_base = OTHER; note = thymine
  modified_base 1691: mod_base = OTHER; note = thymine
  modified_base 1693: mod_base = OTHER; note = thymine
  modified_base 1695..1696: mod_base = OTHER; note = thymine
  modified_base 1705: mod_base = OTHER; note = thymine
  modified_base 1707: mod_base = OTHER; note = thymine
  modified_base 1713: mod_base = OTHER; note = thymine
  modified_base 1720: mod_base = OTHER; note = thymine
  modified_base 1727..1728: mod_base = OTHER; note = thymine
  modified_base 1739: mod_base = OTHER; note = thymine
  modified_base 1741..1743: mod_base = OTHER; note = thymine
  modified_base 1748..1749: mod_base = OTHER; note = thymine
  modified_base 1755..1756: mod_base = OTHER; note = thymine
  modified_base 1758: mod_base = OTHER; note = thymine
  modified_base 1776: mod_base = OTHER; note = thymine
  modified_base 1781..1782: mod_base = OTHER; note = thymine
  modified_base 1786: mod_base = OTHER; note = thymine
  modified_base 1788: mod_base = OTHER; note = thymine
  modified_base 1797: mod_base = OTHER; note = thymine
  modified_base 1806: mod_base = OTHER; note = thymine
  modified_base 1809: mod_base = OTHER; note = thymine
  modified_base 1812: mod_base = OTHER; note = thymine
  modified_base 1825: mod_base = OTHER; note = thymine
  modified_base 1827: mod_base = OTHER; note = thymine
  modified_base 1829: mod_base = OTHER; note = thymine
  modified_base 1836: mod_base = OTHER; note = thymine
  modified_base 1846: mod_base = OTHER; note = thymine
  modified_base 1849: mod_base = OTHER; note = thymine
  modified_base 1854: mod_base = OTHER; note = thymine
  modified_base 1856..1857: mod_base = OTHER; note = thymine
  modified_base 1862: mod_base = OTHER; note = thymine
  modified_base 1866: mod_base = OTHER; note = thymine
  modified_base 1878: mod_base = OTHER; note = thymine
  modified_base 1880: mod_base = OTHER; note = thymine
  modified_base 1887: mod_base = OTHER; note = thymine
  modified_base 1890: mod_base = OTHER; note = thymine
  modified_base 1895..1897: mod_base = OTHER; note = thymine
  modified_base 1899: mod_base = OTHER; note = thymine
  modified_base 1902..1904: mod_base = OTHER; note = thymine
  modified_base 1906: mod_base = OTHER; note = thymine
  modified_base 1910: mod_base = OTHER; note = thymine
  modified_base 1912..1913: mod_base = OTHER; note = thymine
  modified_base 1917: mod_base = OTHER; note = thymine
  modified_base 1927: mod_base = OTHER; note = thymine
  modified_base 1935: mod_base = OTHER; note = thymine
  modified_base 1939: mod_base = OTHER; note = thymine
  modified_base 1948: mod_base = OTHER; note = thymine
  modified_base 1952: mod_base = OTHER; note = thymine
  modified_base 1956: mod_base = OTHER; note = thymine
  modified_base 1963: mod_base = OTHER; note = thymine
  modified_base 1966: mod_base = OTHER; note = thymine
  modified_base 1973: mod_base = OTHER; note = thymine
  modified_base 1977: mod_base = OTHER; note = thymine
  modified_base 1990: mod_base = OTHER; note = thymine
  modified_base 1992: mod_base = OTHER; note = thymine
  modified_base 1994..1996: mod_base = OTHER; note = thymine
  modified_base 1998: mod_base = OTHER; note = thymine
  modified_base 2001..2002: mod_base = OTHER; note = thymine
  modified_base 2007: mod_base = OTHER; note = thymine
  modified_base 2015: mod_base = OTHER; note = thymine
  modified_base 2018..2019: mod_base = OTHER; note = thymine
  modified_base 2038: mod_base = OTHER; note = thymine
  modified_base 2043: mod_base = OTHER; note = thymine
  modified_base 2045: mod_base = OTHER; note = thymine
  modified_base 2048: mod_base = OTHER; note = thymine
  modified_base 2053: mod_base = OTHER; note = thymine
  modified_base 2059: mod_base = OTHER; note = thymine
  modified_base 2062: mod_base = OTHER; note = thymine
  modified_base 2067..2069: mod_base = OTHER; note = thymine
  modified_base 2079: mod_base = OTHER; note = thymine
  modified_base 2086: mod_base = OTHER; note = thymine
  modified_base 2088: mod_base = OTHER; note = thymine
  modified_base 2091: mod_base = OTHER; note = thymine
  modified_base 2100: mod_base = OTHER; note = thymine
  modified_base 2108..2110: mod_base = OTHER; note = thymine
  modified_base 2132: mod_base = OTHER; note = thymine
  modified_base 2134: mod_base = OTHER; note = thymine
  modified_base 2152: mod_base = OTHER; note = thymine
  modified_base 2157: mod_base = OTHER; note = thymine
  modified_base 2166: mod_base = OTHER; note = thymine
  modified_base 2169: mod_base = OTHER; note = thymine
  modified_base 2176: mod_base = OTHER; note = thymine
  modified_base 2182: mod_base = OTHER; note = thymine
  modified_base 2185: mod_base = OTHER; note = thymine
  modified_base 2187..2189: mod_base = OTHER; note = thymine
  modified_base 2195..2196: mod_base = OTHER; note = thymine
  modified_base 2205: mod_base = OTHER; note = thymine
  modified_base 2208: mod_base = OTHER; note = thymine
  modified_base 2210: mod_base = OTHER; note = thymine
  modified_base 2212: mod_base = OTHER; note = thymine
  modified_base 2217: mod_base = OTHER; note = thymine
  modified_base 2223: mod_base = OTHER; note = thymine
  modified_base 2225..2226: mod_base = OTHER; note = thymine
  modified_base 2229: mod_base = OTHER; note = thymine
  modified_base 2233: mod_base = OTHER; note = thymine
  modified_base 2244: mod_base = OTHER; note = thymine
  modified_base 2253: mod_base = OTHER; note = thymine
  modified_base 2256: mod_base = OTHER; note = thymine
  modified_base 2265: mod_base = OTHER; note = thymine
  modified_base 2268..2269: mod_base = OTHER; note = thymine
  modified_base 2272: mod_base = OTHER; note = thymine
  modified_base 2276..2277: mod_base = OTHER; note = thymine
  modified_base 2281: mod_base = OTHER; note = thymine
  modified_base 2286: mod_base = OTHER; note = thymine
  modified_base 2294..2295: mod_base = OTHER; note = thymine
  modified_base 2298: mod_base = OTHER; note = thymine
  modified_base 2304: mod_base = OTHER; note = thymine
  modified_base 2307: mod_base = OTHER; note = thymine
  modified_base 2311: mod_base = OTHER; note = thymine
  modified_base 2313: mod_base = OTHER; note = thymine
  modified_base 2316: mod_base = OTHER; note = thymine
  modified_base 2322: mod_base = OTHER; note = thymine
  modified_base 2329: mod_base = OTHER; note = thymine
  modified_base 2333..2334: mod_base = OTHER; note = thymine
  modified_base 2337: mod_base = OTHER; note = thymine
  modified_base 2345: mod_base = OTHER; note = thymine
  modified_base 2351..2355: mod_base = OTHER; note = thymine
  modified_base 2363: mod_base = OTHER; note = thymine
  modified_base 2373: mod_base = OTHER; note = thymine
  modified_base 2380: mod_base = OTHER; note = thymine
  modified_base 2385: mod_base = OTHER; note = thymine
  modified_base 2391: mod_base = OTHER; note = thymine
  modified_base 2394: mod_base = OTHER; note = thymine
  modified_base 2399: mod_base = OTHER; note = thymine
  modified_base 2402..2403: mod_base = OTHER; note = thymine
  modified_base 2407: mod_base = OTHER; note = thymine
  modified_base 2412: mod_base = OTHER; note = thymine
  modified_base 2422: mod_base = OTHER; note = thymine
  modified_base 2424..2425: mod_base = OTHER; note = thymine
  modified_base 2431: mod_base = OTHER; note = thymine
  modified_base 2445: mod_base = OTHER; note = thymine
  modified_base 2447: mod_base = OTHER; note = thymine
  modified_base 2449: mod_base = OTHER; note = thymine
  modified_base 2451: mod_base = OTHER; note = thymine
  modified_base 2460: mod_base = OTHER; note = thymine
  modified_base 2464: mod_base = OTHER; note = thymine
  modified_base 2466..2469: mod_base = OTHER; note = thymine
  modified_base 2481: mod_base = OTHER; note = thymine
  modified_base 2483: mod_base = OTHER; note = thymine
  modified_base 2486: mod_base = OTHER; note = thymine
  modified_base 2488: mod_base = OTHER; note = thymine
  modified_base 2491: mod_base = OTHER; note = thymine
  modified_base 2496: mod_base = OTHER; note = thymine
  modified_base 2505: mod_base = OTHER; note = thymine
  modified_base 2516..2519: mod_base = OTHER; note = thymine
  modified_base 2523: mod_base = OTHER; note = thymine
  modified_base 2544: mod_base = OTHER; note = thymine
  modified_base 2565: mod_base = OTHER; note = thymine
  modified_base 2572..2574: mod_base = OTHER; note = thymine
  modified_base 2577..2578: mod_base = OTHER; note = thymine
  modified_base 2580..2581: mod_base = OTHER; note = thymine
  modified_base 2584: mod_base = OTHER; note = thymine
  modified_base 2589: mod_base = OTHER; note = thymine
  modified_base 2592: mod_base = OTHER; note = thymine
  modified_base 2599: mod_base = OTHER; note = thymine
  modified_base 2606..2607: mod_base = OTHER; note = thymine
  modified_base 2612: mod_base = OTHER; note = thymine
  modified_base 2616: mod_base = OTHER; note = thymine
  modified_base 2619: mod_base = OTHER; note = thymine
  modified_base 2624: mod_base = OTHER; note = thymine
  modified_base 2629: mod_base = OTHER; note = thymine
  modified_base 2632: mod_base = OTHER; note = thymine
  modified_base 2635: mod_base = OTHER; note = thymine
  modified_base 2643: mod_base = OTHER; note = thymine
  modified_base 2647: mod_base = OTHER; note = thymine
  modified_base 2652: mod_base = OTHER; note = thymine
  modified_base 2670: mod_base = OTHER; note = thymine
  modified_base 2673: mod_base = OTHER; note = thymine
  modified_base 2678..2679: mod_base = OTHER; note = thymine
  modified_base 2682: mod_base = OTHER; note = thymine
  modified_base 2685: mod_base = OTHER; note = thymine
  modified_base 2694: mod_base = OTHER; note = thymine
  modified_base 2698: mod_base = OTHER; note = thymine
  modified_base 2703: mod_base = OTHER; note = thymine
  modified_base 2739: mod_base = OTHER; note = thymine
  modified_base 2747: mod_base = OTHER; note = thymine
  modified_base 2750: mod_base = OTHER; note = thymine
  modified_base 2758..2759: mod_base = OTHER; note = thymine
  modified_base 2761: mod_base = OTHER; note = thymine
  modified_base 2763: mod_base = OTHER; note = thymine
  modified_base 2773: mod_base = OTHER; note = thymine
  modified_base 2787: mod_base = OTHER; note = thymine
  modified_base 2799: mod_base = OTHER; note = thymine
  modified_base 2806: mod_base = OTHER; note = thymine
  modified_base 2808: mod_base = OTHER; note = thymine
  modified_base 2817: mod_base = OTHER; note = thymine
  modified_base 2820: mod_base = OTHER; note = thymine
  modified_base 2829: mod_base = OTHER; note = thymine
  modified_base 2834: mod_base = OTHER; note = thymine
  modified_base 2843..2844: mod_base = OTHER; note = thymine
  modified_base 2862: mod_base = OTHER; note = thymine
  modified_base 2865..2866: mod_base = OTHER; note = thymine
  modified_base 2903..2905: mod_base = OTHER; note = thymine
  modified_base 2913: mod_base = OTHER; note = thymine
  modified_base 2916: mod_base = OTHER; note = thymine
  modified_base 2922: mod_base = OTHER; note = thymine
  modified_base 2924: mod_base = OTHER; note = thymine
  modified_base 2928: mod_base = OTHER; note = thymine
  modified_base 2934: mod_base = OTHER; note = thymine
  modified_base 2937: mod_base = OTHER; note = thymine
  modified_base 2939: mod_base = OTHER; note = thymine
  modified_base 2946: mod_base = OTHER; note = thymine
  modified_base 2949: mod_base = OTHER; note = thymine
  modified_base 2956: mod_base = OTHER; note = thymine
  modified_base 2958..2959: mod_base = OTHER; note = thymine
  modified_base 2961: mod_base = OTHER; note = thymine
  modified_base 2965: mod_base = OTHER; note = thymine
  modified_base 2967: mod_base = OTHER; note = thymine
  modified_base 2978: mod_base = OTHER; note = thymine
  modified_base 2982: mod_base = OTHER; note = thymine
  modified_base 2985: mod_base = OTHER; note = thymine
  modified_base 2990..2991: mod_base = OTHER; note = thymine
  modified_base 2995..2996: mod_base = OTHER; note = thymine
  modified_base 3000: mod_base = OTHER; note = thymine
  modified_base 3007: mod_base = OTHER; note = thymine
  modified_base 3010: mod_base = OTHER; note = thymine
  modified_base 3013: mod_base = OTHER; note = thymine
  modified_base 3018: mod_base = OTHER; note = thymine
  modified_base 3020: mod_base = OTHER; note = thymine
  modified_base 3035: mod_base = OTHER; note = thymine
  modified_base 3042: mod_base = OTHER; note = thymine
  modified_base 3046: mod_base = OTHER; note = thymine
  modified_base 3048: mod_base = OTHER; note = thymine
  modified_base 3057: mod_base = OTHER; note = thymine
  modified_base 3071..3072: mod_base = OTHER; note = thymine
  modified_base 3077: mod_base = OTHER; note = thymine
  modified_base 3081: mod_base = OTHER; note = thymine
  modified_base 3090: mod_base = OTHER; note = thymine
  modified_base 3092: mod_base = OTHER; note = thymine
  modified_base 3097: mod_base = OTHER; note = thymine
  modified_base 3102..3103: mod_base = OTHER; note = thymine
  modified_base 3106: mod_base = OTHER; note = thymine
  modified_base 3112: mod_base = OTHER; note = thymine
  modified_base 3121: mod_base = OTHER; note = thymine
  modified_base 3135: mod_base = OTHER; note = thymine
  modified_base 3147: mod_base = OTHER; note = thymine
  modified_base 3151: mod_base = OTHER; note = thymine
  modified_base 3171..3172: mod_base = OTHER; note = thymine
  modified_base 3180: mod_base = OTHER; note = thymine
  modified_base 3201: mod_base = OTHER; note = thymine
  modified_base 3203: mod_base = OTHER; note = thymine
  modified_base 3205: mod_base = OTHER; note = thymine
  modified_base 3211..3212: mod_base = OTHER; note = thymine
  modified_base 3216: mod_base = OTHER; note = thymine
  modified_base 3221: mod_base = OTHER; note = thymine
  modified_base 3248..3249: mod_base = OTHER; note = thymine
  modified_base 3261..3262: mod_base = OTHER; note = thymine
  modified_base 3288: mod_base = OTHER; note = thymine
  modified_base 3296: mod_base = OTHER; note = thymine
  modified_base 3300: mod_base = OTHER; note = thymine
  modified_base 3314: mod_base = OTHER; note = thymine
  modified_base 3319: mod_base = OTHER; note = thymine
  modified_base 3321: mod_base = OTHER; note = thymine
  modified_base 3324: mod_base = OTHER; note = thymine
  modified_base 3327: mod_base = OTHER; note = thymine
  modified_base 3339: mod_base = OTHER; note = thymine
  modified_base 3342: mod_base = OTHER; note = thymine
  modified_base 3348..3349: mod_base = OTHER; note = thymine
  modified_base 3362: mod_base = OTHER; note = thymine
  modified_base 3364: mod_base = OTHER; note = thymine
  modified_base 3369: mod_base = OTHER; note = thymine
  modified_base 3371..3372: mod_base = OTHER; note = thymine
  modified_base 3387: mod_base = OTHER; note = thymine
  modified_base 3393: mod_base = OTHER; note = thymine
  modified_base 3399: mod_base = OTHER; note = thymine
  modified_base 3407: mod_base = OTHER; note = thymine
  modified_base 3409: mod_base = OTHER; note = thymine
  modified_base 3411: mod_base = OTHER; note = thymine
  modified_base 3419: mod_base = OTHER; note = thymine
  modified_base 3421: mod_base = OTHER; note = thymine
  modified_base 3423: mod_base = OTHER; note = thymine
  modified_base 3432..3433: mod_base = OTHER; note = thymine
  modified_base 3441: mod_base = OTHER; note = thymine
  modified_base 3445..3446: mod_base = OTHER; note = thymine
  modified_base 3449: mod_base = OTHER; note = thymine
  modified_base 3452..3453: mod_base = OTHER; note = thymine
  modified_base 3459: mod_base = OTHER; note = thymine
  modified_base 3461: mod_base = OTHER; note = thymine
  modified_base 3466..3467: mod_base = OTHER; note = thymine
  modified_base 3471: mod_base = OTHER; note = thymine
  modified_base 3474: mod_base = OTHER; note = thymine
  modified_base 3483..3484: mod_base = OTHER; note = thymine
  modified_base 3486: mod_base = OTHER; note = thymine
  modified_base 3489: mod_base = OTHER; note = thymine
  modified_base 3493: mod_base = OTHER; note = thymine
  modified_base 3500: mod_base = OTHER; note = thymine
  modified_base 3522: mod_base = OTHER; note = thymine
  modified_base 3524: mod_base = OTHER; note = thymine
  modified_base 3530: mod_base = OTHER; note = thymine
  modified_base 3533..3534: mod_base = OTHER; note = thymine
  modified_base 3541: mod_base = OTHER; note = thymine
  modified_base 3543: mod_base = OTHER; note = thymine
  modified_base 3547: mod_base = OTHER; note = thymine
  modified_base 3560: mod_base = OTHER; note = thymine
  modified_base 3568: mod_base = OTHER; note = thymine
  modified_base 3573: mod_base = OTHER; note = thymine
  modified_base 3576: mod_base = OTHER; note = thymine
  modified_base 3582: mod_base = OTHER; note = thymine
  modified_base 3585: mod_base = OTHER; note = thymine
  modified_base 3587: mod_base = OTHER; note = thymine
  modified_base 3589: mod_base = OTHER; note = thymine
  modified_base 3591: mod_base = OTHER; note = thymine
  modified_base 3595..3597: mod_base = OTHER; note = thymine
  modified_base 3602: mod_base = OTHER; note = thymine
  modified_base 3605: mod_base = OTHER; note = thymine
  modified_base 3607: mod_base = OTHER; note = thymine
  modified_base 3609: mod_base = OTHER; note = thymine
  modified_base 3617..3619: mod_base = OTHER; note = thymine
  modified_base 3627: mod_base = OTHER; note = thymine
  modified_base 3633: mod_base = OTHER; note = thymine
  modified_base 3642: mod_base = OTHER; note = thymine
  modified_base 3644..3645: mod_base = OTHER; note = thymine
  modified_base 3649: mod_base = OTHER; note = thymine
  modified_base 3668..3669: mod_base = OTHER; note = thymine
  modified_base 3690: mod_base = OTHER; note = thymine
  modified_base 3692: mod_base = OTHER; note = thymine
  modified_base 3700..3701: mod_base = OTHER; note = thymine
  modified_base 3707: mod_base = OTHER; note = thymine
  modified_base 3715: mod_base = OTHER; note = thymine
  modified_base 3732: mod_base = OTHER; note = thymine
  modified_base 3742..3743: mod_base = OTHER; note = thymine
  modified_base 3749: mod_base = OTHER; note = thymine
  modified_base 3754: mod_base = OTHER; note = thymine
  modified_base 3756..3757: mod_base = OTHER; note = thymine
  modified_base 3763..3764: mod_base = OTHER; note = thymine
  modified_base 3769..3770: mod_base = OTHER; note = thymine
  modified_base 3775..3776: mod_base = OTHER; note = thymine
  modified_base 3778: mod_base = OTHER; note = thymine
  modified_base 3783: mod_base = OTHER; note = thymine
  modified_base 3791: mod_base = OTHER; note = thymine
  modified_base 3793: mod_base = OTHER; note = thymine
  modified_base 3796..3797: mod_base = OTHER; note = thymine
  modified_base 3799: mod_base = OTHER; note = thymine
  modified_base 3803: mod_base = OTHER; note = thymine
  modified_base 3805: mod_base = OTHER; note = thymine
  modified_base 3810: mod_base = OTHER; note = thymine
  modified_base 3818: mod_base = OTHER; note = thymine
  modified_base 3824: mod_base = OTHER; note = thymine
  modified_base 3831..3832: mod_base = OTHER; note = thymine
  modified_base 3837: mod_base = OTHER; note = thymine
  modified_base 3839: mod_base = OTHER; note = thymine
  modified_base 3843..3844: mod_base = OTHER; note = thymine
  modified_base 3846: mod_base = OTHER; note = thymine
  modified_base 3849..3850: mod_base = OTHER; note = thymine
  modified_base 3859: mod_base = OTHER; note = thymine
  modified_base 3861: mod_base = OTHER; note = thymine
  modified_base 3863..3864: mod_base = OTHER; note = thymine
  modified_base 3866..3868: mod_base = OTHER; note = thymine
  modified_base 3874: mod_base = OTHER; note = thymine
  modified_base 3881: mod_base = OTHER; note = thymine
  modified_base 3885..3886: mod_base = OTHER; note = thymine
  modified_base 3889..3890: mod_base = OTHER; note = thymine
  modified_base 3896: mod_base = OTHER; note = thymine
  modified_base 3903: mod_base = OTHER; note = thymine
  modified_base 3909: mod_base = OTHER; note = thymine
  modified_base 3915: mod_base = OTHER; note = thymine
  modified_base 3917: mod_base = OTHER; note = thymine
  modified_base 3920..3922: mod_base = OTHER; note = thymine
  modified_base 3925: mod_base = OTHER; note = thymine
  modified_base 3928: mod_base = OTHER; note = thymine
  modified_base 3933: mod_base = OTHER; note = thymine
  modified_base 3941..3942: mod_base = OTHER; note = thymine
  modified_base 3946: mod_base = OTHER; note = thymine
  modified_base 3951..3952: mod_base = OTHER; note = thymine
  modified_base 3954: mod_base = OTHER; note = thymine
  modified_base 3956: mod_base = OTHER; note = thymine
  modified_base 3960: mod_base = OTHER; note = thymine
  modified_base 3964: mod_base = OTHER; note = thymine
  modified_base 3966: mod_base = OTHER; note = thymine
  modified_base 3969..3970: mod_base = OTHER; note = thymine
  modified_base 3972: mod_base = OTHER; note = thymine
  modified_base 3974..3975: mod_base = OTHER; note = thymine
  modified_base 3977: mod_base = OTHER; note = thymine
  modified_base 3984: mod_base = OTHER; note = thymine
  modified_base 3989: mod_base = OTHER; note = thymine
  modified_base 4014..4015: mod_base = OTHER; note = thymine
  modified_base 4023: mod_base = OTHER; note = thymine
  modified_base 4031: mod_base = OTHER; note = thymine
  modified_base 4033..4035: mod_base = OTHER; note = thymine
  modified_base 4040: mod_base = OTHER; note = thymine
  modified_base 4043..4044: mod_base = OTHER; note = thymine
  modified_base 4047..4048: mod_base = OTHER; note = thymine
  modified_base 4051: mod_base = OTHER; note = thymine
  modified_base 4053..4055: mod_base = OTHER; note = thymine
  modified_base 4059: mod_base = OTHER; note = thymine
  modified_base 4061: mod_base = OTHER; note = thymine
  modified_base 4066: mod_base = OTHER; note = thymine
  modified_base 4074: mod_base = OTHER; note = thymine
  modified_base 4076..4077: mod_base = OTHER; note = thymine
  modified_base 4085: mod_base = OTHER; note = thymine
  modified_base 4088..4089: mod_base = OTHER; note = thymine
  modified_base 4094..4095: mod_base = OTHER; note = thymine
  modified_base 4098..4100: mod_base = OTHER; note = thymine
  modified_base 4104: mod_base = OTHER; note = thymine
  modified_base 4106: mod_base = OTHER; note = thymine
  modified_base 4118: mod_base = OTHER; note = thymine
  modified_base 4120..4121: mod_base = OTHER; note = thymine
  modified_base 4124: mod_base = OTHER; note = thymine
  modified_base 4131: mod_base = OTHER; note = thymine
  modified_base 4135: mod_base = OTHER; note = thymine
  modified_base 4140: mod_base = OTHER; note = thymine
  modified_base 4147: mod_base = OTHER; note = thymine
  modified_base 4150: mod_base = OTHER; note = thymine
  modified_base 4153..4155: mod_base = OTHER; note = thymine
  modified_base 4157..4158: mod_base = OTHER; note = thymine
  modified_base 4162: mod_base = OTHER; note = thymine
  modified_base 4165..4167: mod_base = OTHER; note = thymine
  modified_base 4172..4175: mod_base = OTHER; note = thymine
  modified_base 4180..4181: mod_base = OTHER; note = thymine
  modified_base 4183: mod_base = OTHER; note = thymine
  modified_base 4185..4188: mod_base = OTHER; note = thymine
  modified_base 4193: mod_base = OTHER; note = thymine
  modified_base 4198: mod_base = OTHER; note = thymine
  modified_base 4200: mod_base = OTHER; note = thymine
  modified_base 4203: mod_base = OTHER; note = thymine
  modified_base 4205: mod_base = OTHER; note = thymine
  modified_base 4208..4209: mod_base = OTHER; note = thymine
  modified_base 4214: mod_base = OTHER; note = thymine
  modified_base 4218..4219: mod_base = OTHER; note = thymine
  modified_base 4225: mod_base = OTHER; note = thymine
  modified_base 4227..4229: mod_base = OTHER; note = thymine
  modified_base 4233..4235: mod_base = OTHER; note = thymine
  modified_base 4237..4238: mod_base = OTHER; note = thymine
  modified_base 4242..4244: mod_base = OTHER; note = thymine
  modified_base 4246: mod_base = OTHER; note = thymine
  modified_base 4248: mod_base = OTHER; note = thymine
  modified_base 4250: mod_base = OTHER; note = thymine
  modified_base 4252..4253: mod_base = OTHER; note = thymine
  modified_base 4255: mod_base = OTHER; note = thymine
  modified_base 4280: mod_base = OTHER; note = thymine
  modified_base 4287: mod_base = OTHER; note = thymine
  modified_base 4290: mod_base = OTHER; note = thymine
  modified_base 4296..4298: mod_base = OTHER; note = thymine
  modified_base 4307: mod_base = OTHER; note = thymine
  modified_base 4309..4310: mod_base = OTHER; note = thymine
  modified_base 4321..4328: mod_base = OTHER; note = thymine
  modified_base 4346: mod_base = OTHER; note = thymine
  modified_base 4349: mod_base = OTHER; note = thymine
  modified_base 4352: mod_base = OTHER; note = thymine
  modified_base 4357..4358: mod_base = OTHER; note = thymine
  modified_base 4365: mod_base = OTHER; note = thymine
  modified_base 4369: mod_base = OTHER; note = thymine
  modified_base 4371: mod_base = OTHER; note = thymine
  modified_base 4373: mod_base = OTHER; note = thymine
  modified_base 4380: mod_base = OTHER; note = thymine
  modified_base 4384: mod_base = OTHER; note = thymine
  modified_base 4388: mod_base = OTHER; note = thymine
  modified_base 4392: mod_base = OTHER; note = thymine
  modified_base 4412: mod_base = OTHER; note = thymine
  modified_base 4429..4431: mod_base = OTHER; note = thymine
  modified_base 4444: mod_base = OTHER; note = thymine
  modified_base 4448: mod_base = OTHER; note = thymine
  modified_base 4468: mod_base = OTHER; note = thymine
  modified_base 4472: mod_base = OTHER; note = thymine
  modified_base 4484: mod_base = OTHER; note = thymine
  modified_base 4487: mod_base = OTHER; note = thymine
  modified_base 4492: mod_base = OTHER; note = thymine
  modified_base 4494..4497: mod_base = OTHER; note = thymine
  modified_base 4499: mod_base = OTHER; note = thymine
  modified_base 4502..4503: mod_base = OTHER; note = thymine
  modified_base 4509: mod_base = OTHER; note = thymine
  modified_base 4511: mod_base = OTHER; note = thymine
  modified_base 4513: mod_base = OTHER; note = thymine
  modified_base 4518: mod_base = OTHER; note = thymine
  modified_base 4520..4522: mod_base = OTHER; note = thymine
  modified_base 4532: mod_base = OTHER; note = thymine
  modified_base 4536: mod_base = OTHER; note = thymine
  modified_base 4548: mod_base = OTHER; note = thymine
  modified_base 4551: mod_base = OTHER; note = thymine
  modified_base 4560: mod_base = OTHER; note = thymine
  modified_base 4562: mod_base = OTHER; note = thymine
  modified_base 4572..4573: mod_base = OTHER; note = thymine
  modified_base 4586: mod_base = OTHER; note = thymine
  modified_base 4588: mod_base = OTHER; note = thymine
  modified_base 4591: mod_base = OTHER; note = thymine
  modified_base 4594..4595: mod_base = OTHER; note = thymine
  modified_base 4606: mod_base = OTHER; note = thymine
  modified_base 4613: mod_base = OTHER; note = thymine
  modified_base 4618..4619: mod_base = OTHER; note = thymine
  modified_base 4629..4630: mod_base = OTHER; note = thymine
  modified_base 4640: mod_base = OTHER; note = thymine
  modified_base 4643..4645: mod_base = OTHER; note = thymine
  modified_base 4649..4651: mod_base = OTHER; note = thymine
  modified_base 4653..4654: mod_base = OTHER; note = thymine
  modified_base 4658..4659: mod_base = OTHER; note = thymine
  modified_base 4662..4664: mod_base = OTHER; note = thymine
  modified_base 4666: mod_base = OTHER; note = thymine
  modified_base 4674..4675: mod_base = OTHER; note = thymine
  modified_base 4683..4685: mod_base = OTHER; note = thymine
  modified_base 4691: mod_base = OTHER; note = thymine
  modified_base 4697: mod_base = OTHER; note = thymine
  modified_base 4699: mod_base = OTHER; note = thymine
  modified_base 4703: mod_base = OTHER; note = thymine
  modified_base 4711: mod_base = OTHER; note = thymine
  modified_base 4715..4717: mod_base = OTHER; note = thymine
  modified_base 4722..4723: mod_base = OTHER; note = thymine
  modified_base 4728..4730: mod_base = OTHER; note = thymine
  modified_base 4733: mod_base = OTHER; note = thymine
  modified_base 4736..4738: mod_base = OTHER; note = thymine
  modified_base 4747: mod_base = OTHER; note = thymine
  modified_base 4762..4763: mod_base = OTHER; note = thymine
  modified_base 4766..4768: mod_base = OTHER; note = thymine
  modified_base 4772: mod_base = OTHER; note = thymine
  modified_base 4775: mod_base = OTHER; note = thymine
  modified_base 4778..4779: mod_base = OTHER; note = thymine
  modified_base 4784: mod_base = OTHER; note = thymine
  modified_base 4787: mod_base = OTHER; note = thymine
  modified_base 4794: mod_base = OTHER; note = thymine
  modified_base 4800: mod_base = OTHER; note = thymine
  modified_base 4803: mod_base = OTHER; note = thymine
  modified_base 4810..4814: mod_base = OTHER; note = thymine
  modified_base 4820..4822: mod_base = OTHER; note = thymine
  modified_base 4827..4828: mod_base = OTHER; note = thymine
  modified_base 4833: mod_base = OTHER; note = thymine
  modified_base 4839..4840: mod_base = OTHER; note = thymine
  modified_base 4842..4844: mod_base = OTHER; note = thymine
  modified_base 4847: mod_base = OTHER; note = thymine
  modified_base 4850: mod_base = OTHER; note = thymine
  modified_base 4855: mod_base = OTHER; note = thymine
  modified_base 4857..4858: mod_base = OTHER; note = thymine
  modified_base 4860..4861: mod_base = OTHER; note = thymine
  modified_base 4868: mod_base = OTHER; note = thymine
  modified_base 4879: mod_base = OTHER; note = thymine
  modified_base 4884: mod_base = OTHER; note = thymine
  modified_base 4886: mod_base = OTHER; note = thymine
  modified_base 4888: mod_base = OTHER; note = thymine
  modified_base 4890: mod_base = OTHER; note = thymine
  modified_base 4896: mod_base = OTHER; note = thymine
  modified_base 4898..4899: mod_base = OTHER; note = thymine
  modified_base 4901..4904: mod_base = OTHER; note = thymine
  modified_base 4907..4909: mod_base = OTHER; note = thymine
  modified_base 4911: mod_base = OTHER; note = thymine
  modified_base 4918..4921: mod_base = OTHER; note = thymine
  modified_base 4927..4929: mod_base = OTHER; note = thymine
  modified_base 4933: mod_base = OTHER; note = thymine
  modified_base 4935: mod_base = OTHER; note = thymine
  modified_base 4937..4938: mod_base = OTHER; note = thymine
  modified_base 4941..4942: mod_base = OTHER; note = thymine
  modified_base 4949: mod_base = OTHER; note = thymine
  modified_base 4954: mod_base = OTHER; note = thymine
  modified_base 4957..4959: mod_base = OTHER; note = thymine
  modified_base 4968..4970: mod_base = OTHER; note = thymine
  modified_base 4979..4982: mod_base = OTHER; note = thymine
  modified_base 4990: mod_base = OTHER; note = thymine
  modified_base 4992..4993: mod_base = OTHER; note = thymine
  modified_base 4998..5000: mod_base = OTHER; note = thymine
  modified_base 5005..5008: mod_base = OTHER; note = thymine
  modified_base 5010: mod_base = OTHER; note = thymine
  modified_base 5013: mod_base = OTHER; note = thymine
  modified_base 5018: mod_base = OTHER; note = thymine
  modified_base 5020: mod_base = OTHER; note = thymine
  modified_base 5024: mod_base = OTHER; note = thymine
  modified_base 5029: mod_base = OTHER; note = thymine
  modified_base 5031: mod_base = OTHER; note = thymine
  modified_base 5034: mod_base = OTHER; note = thymine
  modified_base 5036: mod_base = OTHER; note = thymine
  modified_base 5039: mod_base = OTHER; note = thymine
  modified_base 5046: mod_base = OTHER; note = thymine
  modified_base 5049..5050: mod_base = OTHER; note = thymine
  modified_base 5081: mod_base = OTHER; note = thymine
  modified_base 5091: mod_base = OTHER; note = thymine
  modified_base 5099..5100: mod_base = OTHER; note = thymine
  modified_base 5102: mod_base = OTHER; note = thymine
  modified_base 5104: mod_base = OTHER; note = thymine
  modified_base 5107: mod_base = OTHER; note = thymine
  modified_base 5115: mod_base = OTHER; note = thymine
  modified_base 5120..5121: mod_base = OTHER; note = thymine
  modified_base 5132: mod_base = OTHER; note = thymine
  modified_base 5134: mod_base = OTHER; note = thymine
  modified_base 5140: mod_base = OTHER; note = thymine
  modified_base 5142: mod_base = OTHER; note = thymine
  modified_base 5151: mod_base = OTHER; note = thymine
  modified_base 5155: mod_base = OTHER; note = thymine
  modified_base 5157: mod_base = OTHER; note = thymine
  modified_base 5159: mod_base = OTHER; note = thymine
  modified_base 5166..5169: mod_base = OTHER; note = thymine
  modified_base 5175: mod_base = OTHER; note = thymine
  modified_base 5178: mod_base = OTHER; note = thymine
  modified_base 5206: mod_base = OTHER; note = thymine
  modified_base 5209: mod_base = OTHER; note = thymine
  modified_base 5212: mod_base = OTHER; note = thymine
  modified_base 5218: mod_base = OTHER; note = thymine
  modified_base 5220..5224: mod_base = OTHER; note = thymine
  modified_base 5226: mod_base = OTHER; note = thymine
  modified_base 5230..5231: mod_base = OTHER; note = thymine
  modified_base 5234: mod_base = OTHER; note = thymine
  modified_base 5236: mod_base = OTHER; note = thymine
  modified_base 5239: mod_base = OTHER; note = thymine
  modified_base 5242: mod_base = OTHER; note = thymine
  modified_base 5245: mod_base = OTHER; note = thymine
  modified_base 5248: mod_base = OTHER; note = thymine
  modified_base 5251..5253: mod_base = OTHER; note = thymine
  modified_base 5255..5256: mod_base = OTHER; note = thymine
  modified_base 5262: mod_base = OTHER; note = thymine
  modified_base 5267: mod_base = OTHER; note = thymine
  modified_base 5273..5276: mod_base = OTHER; note = thymine
  modified_base 5285: mod_base = OTHER; note = thymine
  modified_base 5287: mod_base = OTHER; note = thymine
  modified_base 5300: mod_base = OTHER; note = thymine
  modified_base 5302..5304: mod_base = OTHER; note = thymine
  modified_base 5306..5308: mod_base = OTHER; note = thymine
  modified_base 5310..5314: mod_base = OTHER; note = thymine
  modified_base 5316: mod_base = OTHER; note = thymine
  modified_base 5320: mod_base = OTHER; note = thymine
  modified_base 5324..5325: mod_base = OTHER; note = thymine
  modified_base 5330: mod_base = OTHER; note = thymine
  modified_base 5332: mod_base = OTHER; note = thymine
  modified_base 5334: mod_base = OTHER; note = thymine
  modified_base 5336: mod_base = OTHER; note = thymine
  modified_base 5341: mod_base = OTHER; note = thymine
  modified_base 5344: mod_base = OTHER; note = thymine
  modified_base 5352: mod_base = OTHER; note = thymine
  modified_base 5358: mod_base = OTHER; note = thymine
  modified_base 5361: mod_base = OTHER; note = thymine
  modified_base 5365: mod_base = OTHER; note = thymine
  modified_base 5368..5369: mod_base = OTHER; note = thymine
  modified_base 5373: mod_base = OTHER; note = thymine
  modified_base 5376: mod_base = OTHER; note = thymine
  modified_base 5378..5379: mod_base = OTHER; note = thymine
  modified_base 5393: mod_base = OTHER; note = thymine
  modified_base 5395: mod_base = OTHER; note = thymine
  modified_base 5399: mod_base = OTHER; note = thymine
  modified_base 5401..5402: mod_base = OTHER; note = thymine
  modified_base 5408..5410: mod_base = OTHER; note = thymine
  modified_base 5414: mod_base = OTHER; note = thymine
  modified_base 5416: mod_base = OTHER; note = thymine
  modified_base 5422..5423: mod_base = OTHER; note = thymine
  modified_base 5425..5426: mod_base = OTHER; note = thymine
  modified_base 5430..5435: mod_base = OTHER; note = thymine
  modified_base 5442..5445: mod_base = OTHER; note = thymine
  modified_base 5449..5450: mod_base = OTHER; note = thymine
  modified_base 5453: mod_base = OTHER; note = thymine
  modified_base 5455..5459: mod_base = OTHER; note = thymine
  modified_base 5462: mod_base = OTHER; note = thymine
  modified_base 5476: mod_base = OTHER; note = thymine
  modified_base 5479: mod_base = OTHER; note = thymine
  modified_base 5485: mod_base = OTHER; note = thymine
  modified_base 5492: mod_base = OTHER; note = thymine
  modified_base 5495: mod_base = OTHER; note = thymine
  modified_base 5501: mod_base = OTHER; note = thymine
  modified_base 5505..5506: mod_base = OTHER; note = thymine
  modified_base 5510: mod_base = OTHER; note = thymine
  modified_base 5518: mod_base = OTHER; note = thymine
  modified_base 5522..5523: mod_base = OTHER; note = thymine
  modified_base 5527: mod_base = OTHER; note = thymine
  modified_base 5533: mod_base = OTHER; note = thymine
  modified_base 5537: mod_base = OTHER; note = thymine
  modified_base 5539: mod_base = OTHER; note = thymine
  modified_base 5549: mod_base = OTHER; note = thymine
  modified_base 5554: mod_base = OTHER; note = thymine
  modified_base 5557..5558: mod_base = OTHER; note = thymine
  modified_base 5564..5567: mod_base = OTHER; note = thymine
  modified_base 5582..5585: mod_base = OTHER; note = thymine
  modified_base 5590: mod_base = OTHER; note = thymine
  modified_base 5593: mod_base = OTHER; note = thymine
  modified_base 5600..5603: mod_base = OTHER; note = thymine
  modified_base 5608..5609: mod_base = OTHER; note = thymine
  modified_base 5611: mod_base = OTHER; note = thymine
  modified_base 5614: mod_base = OTHER; note = thymine
  modified_base 5616: mod_base = OTHER; note = thymine
  modified_base 5618: mod_base = OTHER; note = thymine
  modified_base 5626: mod_base = OTHER; note = thymine
  modified_base 5628..5629: mod_base = OTHER; note = thymine
  modified_base 5631: mod_base = OTHER; note = thymine
  modified_base 5636: mod_base = OTHER; note = thymine
  modified_base 5638..5639: mod_base = OTHER; note = thymine
  modified_base 5659: mod_base = OTHER; note = thymine
  modified_base 5663: mod_base = OTHER; note = thymine
  modified_base 5671: mod_base = OTHER; note = thymine
  modified_base 5676: mod_base = OTHER; note = thymine
  modified_base 5678..5679: mod_base = OTHER; note = thymine
  modified_base 5681: mod_base = OTHER; note = thymine
  modified_base 5687: mod_base = OTHER; note = thymine
  modified_base 5691..5692: mod_base = OTHER; note = thymine
  modified_base 5695..5696: mod_base = OTHER; note = thymine
  modified_base 5700: mod_base = OTHER; note = thymine
  modified_base 5703: mod_base = OTHER; note = thymine
  modified_base 5710: mod_base = OTHER; note = thymine
  modified_base 5723: mod_base = OTHER; note = thymine
  modified_base 5725..5726: mod_base = OTHER; note = thymine
  modified_base 5732: mod_base = OTHER; note = thymine
  modified_base 5737: mod_base = OTHER; note = thymine
  modified_base 5743: mod_base = OTHER; note = thymine
  modified_base 5751..5752: mod_base = OTHER; note = thymine
  modified_base 5754: mod_base = OTHER; note = thymine
  modified_base 5759: mod_base = OTHER; note = thymine
  modified_base 5762: mod_base = OTHER; note = thymine
  modified_base 5767: mod_base = OTHER; note = thymine
  modified_base 5773: mod_base = OTHER; note = thymine
  modified_base 5777: mod_base = OTHER; note = thymine
  modified_base 5780: mod_base = OTHER; note = thymine
  modified_base 5786: mod_base = OTHER; note = thymine
  modified_base 5796..5797: mod_base = OTHER; note = thymine
  modified_base 5800..5801: mod_base = OTHER; note = thymine
  modified_base 5803: mod_base = OTHER; note = thymine
  modified_base 5812: mod_base = OTHER; note = thymine
  modified_base 5830: mod_base = OTHER; note = thymine
  modified_base 5837..5842: mod_base = OTHER; note = thymine
  modified_base 5851: mod_base = OTHER; note = thymine
  modified_base 5858: mod_base = OTHER; note = thymine
  modified_base 5861: mod_base = OTHER; note = thymine
  modified_base 5863: mod_base = OTHER; note = thymine
  modified_base 5867: mod_base = OTHER; note = thymine
  modified_base 5872..5873: mod_base = OTHER; note = thymine
  modified_base 5876: mod_base = OTHER; note = thymine
  modified_base 5879..5880: mod_base = OTHER; note = thymine
  modified_base 5893: mod_base = OTHER; note = thymine
  modified_base 5897: mod_base = OTHER; note = thymine
  modified_base 5905: mod_base = OTHER; note = thymine
  modified_base 5921: mod_base = OTHER; note = thymine
  modified_base 5932: mod_base = OTHER; note = thymine
  modified_base 5936: mod_base = OTHER; note = thymine
  modified_base 5938: mod_base = OTHER; note = thymine
  modified_base 5944: mod_base = OTHER; note = thymine
  modified_base 5955..5956: mod_base = OTHER; note = thymine
  modified_base 5965: mod_base = OTHER; note = thymine
  modified_base 5967..5968: mod_base = OTHER; note = thymine
  modified_base 5972: mod_base = OTHER; note = thymine
  modified_base 5980: mod_base = OTHER; note = thymine
  modified_base 5983..5984: mod_base = OTHER; note = thymine
  modified_base 5987: mod_base = OTHER; note = thymine
  modified_base 5989: mod_base = OTHER; note = thymine
  modified_base 5993..5994: mod_base = OTHER; note = thymine
  modified_base 6006..6007: mod_base = OTHER; note = thymine
  modified_base 6010: mod_base = OTHER; note = thymine
  modified_base 6015: mod_base = OTHER; note = thymine
  modified_base 6019: mod_base = OTHER; note = thymine
  modified_base 6029: mod_base = OTHER; note = thymine
  modified_base 6034..6035: mod_base = OTHER; note = thymine
  modified_base 6046..6047: mod_base = OTHER; note = thymine
  modified_base 6049: mod_base = OTHER; note = thymine
  modified_base 6054: mod_base = OTHER; note = thymine
  modified_base 6061..6062: mod_base = OTHER; note = thymine
  modified_base 6068: mod_base = OTHER; note = thymine
  modified_base 6072: mod_base = OTHER; note = thymine
  modified_base 6075..6077: mod_base = OTHER; note = thymine
  modified_base 6079..6080: mod_base = OTHER; note = thymine
  modified_base 6083: mod_base = OTHER; note = thymine
  modified_base 6086: mod_base = OTHER; note = thymine
  modified_base 6090: mod_base = OTHER; note = thymine
  modified_base 6092: mod_base = OTHER; note = thymine
  modified_base 6101: mod_base = OTHER; note = thymine
  modified_base 6107: mod_base = OTHER; note = thymine
  modified_base 6119: mod_base = OTHER; note = thymine
  modified_base 6121: mod_base = OTHER; note = thymine
  modified_base 6124..6125: mod_base = OTHER; note = thymine
  modified_base 6133: mod_base = OTHER; note = thymine
  modified_base 6143: mod_base = OTHER; note = thymine
  modified_base 6146: mod_base = OTHER; note = thymine
  modified_base 6153: mod_base = OTHER; note = thymine
  modified_base 6158: mod_base = OTHER; note = thymine
  modified_base 6160: mod_base = OTHER; note = thymine
  modified_base 6163: mod_base = OTHER; note = thymine
  modified_base 6166..6167: mod_base = OTHER; note = thymine
  modified_base 6169: mod_base = OTHER; note = thymine
  modified_base 6171: mod_base = OTHER; note = thymine
  modified_base 6185: mod_base = OTHER; note = thymine
  modified_base 6194: mod_base = OTHER; note = thymine
  modified_base 6196: mod_base = OTHER; note = thymine
  modified_base 6200: mod_base = OTHER; note = thymine
  modified_base 6209: mod_base = OTHER; note = thymine
  modified_base 6217: mod_base = OTHER; note = thymine
  modified_base 6221: mod_base = OTHER; note = thymine
  modified_base 6226: mod_base = OTHER; note = thymine
  modified_base 6230: mod_base = OTHER; note = thymine
  modified_base 6234: mod_base = OTHER; note = thymine
  modified_base 6238: mod_base = OTHER; note = thymine
  modified_base 6241..6242: mod_base = OTHER; note = thymine
  modified_base 6248..6249: mod_base = OTHER; note = thymine
  modified_base 6252: mod_base = OTHER; note = thymine
  modified_base 6256: mod_base = OTHER; note = thymine
  modified_base 6258: mod_base = OTHER; note = thymine
  modified_base 6268..6270: mod_base = OTHER; note = thymine
  modified_base 6273: mod_base = OTHER; note = thymine
  modified_base 6276: mod_base = OTHER; note = thymine
  modified_base 6278: mod_base = OTHER; note = thymine
  modified_base 6280: mod_base = OTHER; note = thymine
  modified_base 6283..6285: mod_base = OTHER; note = thymine
  modified_base 6289..6290: mod_base = OTHER; note = thymine
  modified_base 6293..6295: mod_base = OTHER; note = thymine
  modified_base 6301..6302: mod_base = OTHER; note = thymine
  modified_base 6305..6309: mod_base = OTHER; note = thymine
  modified_base 6312..6314: mod_base = OTHER; note = thymine
  modified_base 6322: mod_base = OTHER; note = thymine
  modified_base 6324: mod_base = OTHER; note = thymine
  modified_base 6328: mod_base = OTHER; note = thymine
  modified_base 6334: mod_base = OTHER; note = thymine
  modified_base 6337..6341: mod_base = OTHER; note = thymine
  modified_base 6344: mod_base = OTHER; note = thymine
  modified_base 6347: mod_base = OTHER; note = thymine
  modified_base 6349: mod_base = OTHER; note = thymine
  modified_base 6352: mod_base = OTHER; note = thymine
  modified_base 6361: mod_base = OTHER; note = thymine
  modified_base 6365..6366: mod_base = OTHER; note = thymine
  modified_base 6371: mod_base = OTHER; note = thymine
  modified_base 6375..6378: mod_base = OTHER; note = thymine
  modified_base 6381..6382: mod_base = OTHER; note = thymine
  modified_base 6387: mod_base = OTHER; note = thymine
  modified_base 6393: mod_base = OTHER; note = thymine
  modified_base 6403: mod_base = OTHER; note = thymine
  modified_base 6412: mod_base = OTHER; note = thymine
  modified_base 6420: mod_base = OTHER; note = thymine
  modified_base 6422..6423: mod_base = OTHER; note = thymine
  modified_base 6425..6426: mod_base = OTHER; note = thymine
  modified_base 6431: mod_base = OTHER; note = thymine
  modified_base 6434..6440: mod_base = OTHER; note = thymine
  modified_base 6442: mod_base = OTHER; note = thymine
  modified_base 6448: mod_base = OTHER; note = thymine
  modified_base 6451: mod_base = OTHER; note = thymine
  modified_base 6453: mod_base = OTHER; note = thymine
  modified_base 6456: mod_base = OTHER; note = thymine
  modified_base 6459..6460: mod_base = OTHER; note = thymine
  modified_base 6481: mod_base = OTHER; note = thymine
  modified_base 6490: mod_base = OTHER; note = thymine
  modified_base 6493..6495: mod_base = OTHER; note = thymine
  modified_base 6497..6499: mod_base = OTHER; note = thymine
  modified_base 6506: mod_base = OTHER; note = thymine
  modified_base 6514: mod_base = OTHER; note = thymine
  modified_base 6521: mod_base = OTHER; note = thymine
  modified_base 6523..6527: mod_base = OTHER; note = thymine
  modified_base 6535: mod_base = OTHER; note = thymine
  modified_base 6539: mod_base = OTHER; note = thymine
  modified_base 6543..6544: mod_base = OTHER; note = thymine
  modified_base 6559: mod_base = OTHER; note = thymine
  modified_base 6566: mod_base = OTHER; note = thymine
  modified_base 6569: mod_base = OTHER; note = thymine
  modified_base 6571..6572: mod_base = OTHER; note = thymine
  modified_base 6574..6575: mod_base = OTHER; note = thymine
  modified_base 6577: mod_base = OTHER; note = thymine
  modified_base 6580: mod_base = OTHER; note = thymine
  modified_base 6582: mod_base = OTHER; note = thymine
  modified_base 6588: mod_base = OTHER; note = thymine
  modified_base 6591..6592: mod_base = OTHER; note = thymine
  modified_base 6603..6604: mod_base = OTHER; note = thymine
  modified_base 6612: mod_base = OTHER; note = thymine
  modified_base 6614: mod_base = OTHER; note = thymine
  modified_base 6616: mod_base = OTHER; note = thymine
  modified_base 6626: mod_base = OTHER; note = thymine
  modified_base 6630: mod_base = OTHER; note = thymine
  modified_base 6634: mod_base = OTHER; note = thymine
  modified_base 6638: mod_base = OTHER; note = thymine
  modified_base 6640: mod_base = OTHER; note = thymine
  modified_base 6643: mod_base = OTHER; note = thymine
  modified_base 6646: mod_base = OTHER; note = thymine
  modified_base 6649: mod_base = OTHER; note = thymine
  modified_base 6651..6652: mod_base = OTHER; note = thymine
  modified_base 6658: mod_base = OTHER; note = thymine
  modified_base 6662: mod_base = OTHER; note = thymine
  modified_base 6665: mod_base = OTHER; note = thymine
  modified_base 6671: mod_base = OTHER; note = thymine
  modified_base 6677: mod_base = OTHER; note = thymine
  modified_base 6681: mod_base = OTHER; note = thymine
  modified_base 6684: mod_base = OTHER; note = thymine
  modified_base 6686: mod_base = OTHER; note = thymine
  modified_base 6688..6689: mod_base = OTHER; note = thymine
  modified_base 6696..6697: mod_base = OTHER; note = thymine
  modified_base 6702: mod_base = OTHER; note = thymine
  modified_base 6711: mod_base = OTHER; note = thymine
  modified_base 6714..6715: mod_base = OTHER; note = thymine
  modified_base 6722: mod_base = OTHER; note = thymine
  modified_base 6735: mod_base = OTHER; note = thymine
  modified_base 6741: mod_base = OTHER; note = thymine
  modified_base 6752..6753: mod_base = OTHER; note = thymine
  modified_base 6756: mod_base = OTHER; note = thymine
  modified_base 6771..6772: mod_base = OTHER; note = thymine
  modified_base 6786: mod_base = OTHER; note = thymine
  modified_base 6796: mod_base = OTHER; note = thymine
  modified_base 6801: mod_base = OTHER; note = thymine
  modified_base 6805: mod_base = OTHER; note = thymine
  modified_base 6812: mod_base = OTHER; note = thymine
  modified_base 6817: mod_base = OTHER; note = thymine
  modified_base 6819: mod_base = OTHER; note = thymine
  modified_base 6835..6836: mod_base = OTHER; note = thymine
  modified_base 6861: mod_base = OTHER; note = thymine
  modified_base 6863: mod_base = OTHER; note = thymine
  modified_base 6868: mod_base = OTHER; note = thymine
  modified_base 6880: mod_base = OTHER; note = thymine
  modified_base 6907..6908: mod_base = OTHER; note = thymine
  modified_base 6924: mod_base = OTHER; note = thymine
  modified_base 6927: mod_base = OTHER; note = thymine
  modified_base 6929: mod_base = OTHER; note = thymine
  modified_base 6931..6933: mod_base = OTHER; note = thymine
  modified_base 6935: mod_base = OTHER; note = thymine
  modified_base 6938: mod_base = OTHER; note = thymine
  modified_base 6941: mod_base = OTHER; note = thymine
  modified_base 6943: mod_base = OTHER; note = thymine
  modified_base 6948..6950: mod_base = OTHER; note = thymine
  modified_base 6958: mod_base = OTHER; note = thymine
  modified_base 6960: mod_base = OTHER; note = thymine
  modified_base 6964..6965: mod_base = OTHER; note = thymine
  modified_base 6971: mod_base = OTHER; note = thymine
  modified_base 6975..6979: mod_base = OTHER; note = thymine
  modified_base 6981: mod_base = OTHER; note = thymine
  modified_base 6984: mod_base = OTHER; note = thymine
  modified_base 6987: mod_base = OTHER; note = thymine
  modified_base 6990: mod_base = OTHER; note = thymine
  modified_base 7006: mod_base = OTHER; note = thymine
  modified_base 7008: mod_base = OTHER; note = thymine
  modified_base 7032..7036: mod_base = OTHER; note = thymine
  modified_base 7041..7042: mod_base = OTHER; note = thymine
ctggccttttgctggccttttgctcacatgtcctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagagcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgaccggtgccaccatgagccccaagaagaagagaaaggtggaggccagcatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattccggatccggacctaagaaaaagaggaaggtggcggccgcttacccatacgatgttccagattacgcttacccttatgacgtaccagactatgcttatccctatgacgtaccggactacgcctgaaagcttcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattggaccggtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaacccctaccaactggtcggggtttgaaacgggtcttcgagaagaccttttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttc